(19) United States
(12) Patent Application Publication
(10) Pub. No.: US 2014/0315883 A1
(43) Pub. Date: Oct. 23, 2014

(54) 6H-IMIDAZO[1,5-a]PYRROLO[2,3-e]PYRAZINE COMPOUNDS

(71) Applicants: Eric Breinlinger, Charlton, MA (US); Heather Davis, Oxford, MA (US); Michael Hoemann, Shrewsbury, MA (US); Bin Li, Ashland, MA (US); Biqin Li, Southborough, MA (US); Gagandeep Somal, Ashland, MA (US); Stacy Van Epps, Ashland, MA (US); Lu Wang, Westborough, MA (US); Lu Wang, Northborough, MA (US)

(72) Inventors: Eric Breinlinger, Charlton, MA (US); Heather Davis, Oxford, MA (US); Michael Hoemann, Shrewsbury, MA (US); Bin Li, Ashland, MA (US); Biqin Li, Southborough, MA (US); Gagandeep Somal, Ashland, MA (US); Stacy Van Epps, Ashland, MA (US); Lu Wang, Westborough, MA (US); Lu Wang, Northborough, MA (US)

(73) Assignee: ABBVIE INC., North Chicago, IL (US)

(21) Appl. No.: 14/256,224

(22) Filed: Apr. 18, 2014

(30) Foreign Application Priority Data
Apr. 19, 2013 (CN) PCT/CN2013/074405

(51) Int. Cl. C07D 487/16

(52) U.S. Cl. CPC C07D 487/16
USPC 514/210.18; 544/346; 514/250; 544/115; 514/233.2; 544/105; 514/230.5

(57) ABSTRACT

The invention provides a compound of Formula (I as defined herein, pharmaceutically acceptable salts, pro-drugs, biologically active metabolites, stereoisomers and isomers thereof wherein the variables are defined herein. The compounds of the invention are useful for treating immunological and oncological conditions.

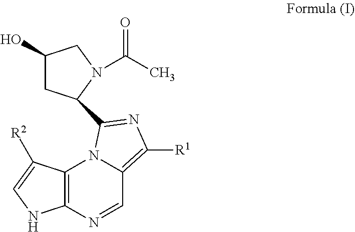

### REFERENCE TO RELATED APPLICATION

This is a U.S. national application filed under 35 U.S.C. §111(a), claiming the benefit of priority under 35 U.S.C. §365(a) to International Application No. PCT/CN2013/074405, filed on Apr. 19, 2013, which designates at least one country other than the United States. The entire content of said International application is incorporated herein by reference.

### BACKGROUND OF THE INVENTION

The invention provides a novel class of compounds, pharmaceutical compositions comprising such compounds and methods of using such compounds to treat or prevent diseases or disorders associated with abnormal or deregulated kinase activity, particularly diseases or disorders that involve abnormal activation of the Syk kinase.

The protein kinases represent a large family of proteins that play a central role in the regulation of a wide variety of cellular processes and maintenance of cellular function. Spleen tyrosine kinase (Syk) (J. Bio. Chem., 1991, 266:15790) is a non-receptor tyrosine kinase that plays a key role in immunoreceptor signaling in a host of inflammatory cells including B cells, mast cells, macrophages and neutrophils. Syk is related to zeta associated protein 70 (ZAP-70) but also demonstrates similarity with JAK, Src and Tec family kinases.

Syk plays a critical and specific role in B-cell receptor (BCR) signaling on auto-reactive B cells and in FcR signaling on mast cells, macrophages, osteoclasts and neutrophils. (see Immunology Today, 2002, 21(3):148; and Current Opinion in Immunology, 2002, 14(3):341). Syk plays a key role in the activation mediated by Fc receptors of sentinel cells (mast cells and macrophages) and effector cells (neutrophils, basophils and eosinophils). The importance of Syk in rheumatoid arthritis is substantiated by data demonstrating the importance of Fc receptors (FcR) function and immune complexes in disease pathogenesis. Syk also mediates the activation of B cells through the BCR, which results in their expansion and the production of antispecific immunoglobulins. Therefore any disease that revolves around antibody-Fc receptor interactions may be modulated by Syk suppression. Thus a Syk inhibitor is likely to dampen both the initiation of the disease by blocking BCR signaling and the effector phase of the disease by blocking FcR signaling on macrophages, neutrophils and mast cells. Furthermore, blocking Syk would provide the added benefit of inhibiting osteoclast maturation and therefore attenuate bony erosions, joint destruction and generalized osteopenia associated with rheumatoid arthritis. Moreover Syk acts upstream close to the receptors at the initiation of complex signaling events and thus its inhibition influences all responses elicited by the activating agent. In mast cells for example, inhibition of Syk blocks the early release of a number of granule contents, as well as the subsequent production and secretion of lipid mediators and cytokines. Syk inhibitors can thus impart multiple beneficial effects as each of these mediators play distinct roles in the integrated inflammatory response.

Inhibiting Syk should impact several critical nodes of the inflammatory cascade resulting in an effective and rapid suppression of the deleterious responses. Inhibiting Syk may be useful in treating a host of inflammatory and allergic diseases—for example (but not limited to), rheumatoid arthritis (RA), systemic lupus erythematosus (SLE), multiple sclerosis (MS) and type I hypersensitivity reactions such as allergic rhinitis, allergic conjunctivitis, atopic dermatitis, allergic asthma and systemic anaphylaxis. For a review on targeting Syk as a treatment of autoimmune and allergic disorders, see Expert Opin. Invest. Drugs, 2004, 13(7):743.

Taken together, Syk inhibitors provide a broad modality to treat a host of inflammatory diseases and immunological disorders.

### SUMMARY OF THE INVENTION

In a first embodiment the invention provides a compound of Formula (I)

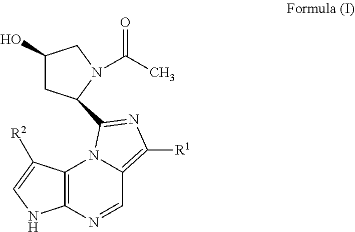

or a pro-drug, metabolite, isomer, stereoisomer or pharmaceutically acceptable salt thereof wherein

R¹ is phenyl, 2,3-dihydorbenzo[b][1,4]dioxinyl, thiazolyl, thienyl, or 1,2,3,4-tetrahydroquinolinyl, wherein

the phenyl is substituted by one or more substituents independently selected from —C(CH₃)₂OH, —OCH₃, —C(CH₃)₂CN, —OCH₂CH₂OCH₃, —OCH₂CH₂-morpholinyl, —CH₃, morpholinyl, F, —OCH(CH₃)₂, —OCF₃, —OCH₂CF₃, 1-hydroxycyclobuty, —OCH₂-cyclopropyl, tetrahydropyranyl, —CH₂CN, —CHF₂, CF₃, piperidinyl, —N(H)CH(CH₃)₂, 2-methylmorpholinyl, 4,4-difluoropiperidinyl, 4,4-difluoroazetidinyl, —C(O)-4,4-difluropiperidinyl, —N(H)CH₃, —N(H)CH₂CH₃, —C(H)(CH₃)OCH₃, —OCH₂CH₂-triazolyl, —OCH₂-3-methyloxetanyl, —CH₂-triazolyl or —OCH(CH₃)-cyclopropyl; or

the thienyl is substituted by —OCH₃ or —CH₂OH; or

the thiazolyl is substituted by tert-butyl; and

R² is H, Cl or CH₃.

In a second embodiment the invention provides a compound of Formula (I) wherein the compound is
1-((2R,4R)-4-hydroxy-2-(3-(4-(2-hydroxypropan-2-yl)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-methoxyphenyl)-6H-imidazo[1,5-a]pyrrolo[2,3-e]pyrazin-1-yl)pyrrolidin-1-yl)ethanone;
1-((2R,4R)-4-hydroxy-2-(3-(4-methoxy-3-methylphenyl)-6H-imidazo[1,5-a]pyrrolo[2,3-e]pyrazin-1-yl)pyrrolidin-1-yl)ethanone;
2-(4-(1-((2R,4R)-1-acetyl-4-hydroxypyrrolidin-2-yl)-6H-imidazo[1,5-a]pyrrolo[2,3-e]pyrazin-3-yl)phenyl)-2-methylpropanenitrile;
1-((2R,4R)-2-(3-(3,4-dimethoxyphenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(2-methoxyethoxy)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-(2-morpholinoethoxy)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-morpholinophenyl)-6H-imidazo[1,5-a]pyrrolo[2,3-e]pyrazin-1-yl)pyrrolidin-1-yl)ethanone;
1-((2R,4R)-2-(3-(3-fluoro-4-(2-hydroxypropan-2-yl)phenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-isopropoxyphenyl)-6H-imidazo[1,5-a]pyrrolo[2,3-e]pyrazin-1-yl)pyrrolidin-1-yl)ethanone;
1-((2R,4R)-2-(3-(benzo[d][1,3]dioxol-5-yl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(trifluoromethoxy)phenyl)-6H-imidazo[1,5-a]pyrrolo[2,3-e]pyrazin-1-yl)pyrrolidin-1-yl)ethanone;
1-((2R,4R)-2-(3-(3-fluoro-4-methoxyphenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(2,2,2-trifluoroethoxy)phenyl)-6H-imidazo[1,5-a]pyrrolo[2,3-e]pyrazin-1-yl)pyrrolidin-1-yl)ethanone;
1-((2R,4R)-4-hydroxy-2-(3-(5-methoxythiophen-2-yl)-6H-imidazo[1,5-a]pyrrolo[2,3-e]pyrazin-1-yl)pyrrolidin-1-yl)ethanone;
1-((2R,4R)-4-hydroxy-2-(3-(4-(1-hydroxycyclobutyl)phenyl)-6H-imidazo[1,5-a]pyrrolo[2,3-e]pyrazin-1-yl)pyrrolidin-1-yl)ethanone;
1-((2R,4R)-2-(3-(4-(cyclopropylmethoxy)phenyl)-6H-imidazo[1,5-a]pyrrolo[2,3-e]pyrazin-1-yl)-4-hydroxypyrrolidin-1-yl)ethanone;
1-((2R,4R)-2-(3-(4-ethoxy-3-methylphenyl)-6H-imidazo[1,5-a]pyrrolo[2,3-e]pyrazin-1-yl)-4-hydroxypyrrolidin-1-yl)ethanone;
1-((2R,4R)-2-(3-(4-ethoxyphenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(methoxymethyl)phenyl)-6H-imidazo[1,5-a]pyrrolo[2,3-e]pyrazin-1-yl)pyrrolidin-1-yl)ethanone;
1-((2R,4R)-2-(3-(2-(tert-Butyl)thiazol-5-yl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(tetrahydro-2H-pyran-4-yl)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-((3-methyloxetan-3-yl)methoxy)phenyl)-6H-imidazo[1,5-a]pyrrolo[2,3-e]pyrazin-1-yl)pyrrolidin-1-yl)ethanone;
1-((2R,4R)-2-(3-(3,4-dihydro-2H-benzo[b][1,4]oxazin-6-yl)-6H-imidazo[1,5-a]pyrrolo[2,3-e]pyrazin-1-yl)-4-hydroxypyrrolidin-1-yl)ethanone;
1-((2R,4R)-2-(3-(2,3-dihydrobenzo[b][1,4]dioxin-6-yl)-6H-imidazo[1,5-a]pyrrolo[2,3-e]pyrazin-1-yl)-4-hydroxypyrrolidin-1-yl)ethanone;
1-((2S,4S)-4-hydroxy-2-(3-(thiophen-3-yl)-6H-imidazo[1,5-a]pyrrolo[2,3-e]pyrazin-1-yl)pyrrolidin-1-yl)ethanone;
2-(4-(1-((2S,4S)-1-acetyl-4-hydroxypyrrolidin-2-yl)-6H-imidazo[1,5-a]pyrrolo[2,3-e]pyrazin-3-yl)phenyl)acetonitrile;
1-((2R,4R)-4-hydroxy-2-(3-(1,2,3,4-tetrahydroquinolin-7-yl)-6H-imidazo[1,5-a]pyrrolo[2,3-e]pyrazin-1-yl)pyrrolidin-1-yl)ethanone;
1-((2R,4R)-2-(3-(chroman-6-yl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(piperidin-1-yl)phenyl)-6H-imidazo[1,5-a]pyrrolo[2,3-e]pyrazin-1-yl)pyrrolidin-1-yl)ethanone;
1-((2R,4R)-2-(3-(4-(difluoromethyl)phenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(trifluoromethyl)phenyl)-6H-imidazo[1,5-a]pyrrolo[2,3-e]pyrazin-1-yl)pyrrolidin-1-yl)ethanone;
1-((2R,4R)-2-(8-chloro-3-(4-morpholinophenyl)-6H-imidazo[1,5-a]pyrrolo[2,3-e]pyrazin-1-yl)-4-hydroxypyrrolidin-1-yl)ethanone;
1-((2R,4R)-2-(8-chloro-3-(4-ethoxyphenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(isopropylamino)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-(2-methylmorpholino)phenyl)-6H-imidazo[1,5-a]pyrrolo[2,3-e]pyrazin-1-yl)pyrrolidin-1-yl)ethanone;
1-((2R,4R)-2-(3-(4-(4,4-difluoropiperidin-1-yl)phenyl)-6H-imidazo[1,5-a]pyrrolo[2,3-e]pyrazin-1-yl)-4-hydroxypyrrolidin-1-yl)ethanone;
1-((2R,4R)-2-(3-(4-(3,3-difluoroazetidin-1-yl)phenyl)-6H-imidazo[1,5-a]pyrrolo[2,3-e]pyrazin-1-yl)-4-hydroxypyrrolidin-1-yl)ethanone;
(2R,4R)-2-(3-(2,3-Dihydrobenzo[b][1,4]dioxin-6-yl)-6H-imidazo[1,5-c]pyrrolo[2,3-e]pyrazin-1-yl)-4-hydroxypyrrolidine-1-carboxamide;
2-fluoro-1-((2R,4R)-4-hydroxy-2-(3-(4-(2-methoxyethoxy)phenyl)-6H-imidazo[1,5-a]pyrrolo[2,3-e]pyrazin-1-yl)pyrrolidin-1-yl)ethanone;
1-((2R,4R)-2-(3-(4-(4,4-difluoropiperidine-1-carbonyl)phenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(isopropylamino)phenyl)-8-methyl-6H-imidazo[1,5-a]pyrrolo[2,3-e]pyrazin-1-yl)pyrrolidin-1-yl)ethanone;
1-((2R,4R)-4-hydroxy-2-(3-(4-(methylamino)phenyl)-6H-imidazo[1,5-a]pyrrolo[2,3-e]pyrazin-1-yl)pyrrolidin-1-yl)ethanone;
1-((2R,4R)-2-(3-(4-(ethylamino)phenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-((R)-1-methoxyethyl)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-((S)-1-methoxyethyl)phenyl)-6H-imidazo[1,5-a]pyrrolo[2,3-e]pyrazin-1-yl)pyrrolidin-1-yl)ethanone;
1-((2R,4R)-4-hydroxy-2-(3-(5-(hydroxymethyl)thiophen-3-yl)-6H-imidazo[1,5-a]pyrrolo[2,3-e]pyrazin-1-yl)pyrrolidin-1-yl)ethanone;
1-((2R,4R)-2-(3-(4-(2-(2H-1,2,3-triazol-2-yl)ethoxy)phenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-((S)-2-methylmorpholino)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-((R)-2-methylmorpholino)phenyl)-6H-imidazo[1,5-a]pyrrolo[2,3-e]pyrazin-1-yl)pyrrolidin-1-yl)ethanone;
1-((2R,4R)-2-(3-(4-((2H-1,2,3-triazol-2-yl)methyl)phenyl)-6H-imidazo[1,5-a]pyrrolo[2,3-e]pyrazin-1-yl)-4-hydroxypyrrolidin-1-yl)ethanone; or,
1-((2R,4R)-2-(3-(4-((R)-1-cyclopropylethoxy)phenyl)-6H-imidazo[1,5-a]pyrrolo[2,3-e]pyrazin-1-yl)-4-hydroxypyrrolidin-1-yl)ethanone.

In a third embodiment the invention provides a method of treating a disease comprising administering a therapeutically effective amount of a compound of Formula (I) to a patient in need thereof.

In a fourth embodiment the invention provides a method according to the third embodiment wherein the disease is rheumatoid arthritis, juvenile rheumatoid arthritis, osteoarthritis, Crohn's disease, inflammatory bowel disease, ankylosing spondylitis, interstitial cystitis, asthma, systemic lupus erythematosus or multiple sclerosis.

In a fifth embodiment the invention provides a kit comprising a packaged product comprising components with which to administer a compound of Formula (I) of the invention for treatment of an autoimmune disorder.

In a sixth embodiment the invention provides a kit according to the fifth embodiment wherein the packaged product comprises a compound of Formula (I) and instructions for use.

In a seventh embodiment the invention provides a pharmaceutical composition comprising a compound according to claim 1 and one or more pharmaceutically acceptable excipients.

### DETAILED DESCRIPTION OF THE INVENTION

Protein kinases are a broad and diverse class, of over 500 enzymes, that include oncogenes, growth factors receptors, signal transduction intermediates, apoptosis related kinases and cyclin dependent kinases. They are responsible for the transfer of a phosphate group to specific tyrosine, serine or threonine amino acid residues, and are broadly classified as tyrosine and serine/threonine kinases as a result of their substrate specificity.

Spleen Tyrosine Kinase (Syk) is a 72 kDa non-receptor protein tyrosine kinase that functions as a key signaling regulator in most hematopoietic cells. Its closest homolog is zeta-associated protein 70 (ZAP-70). Like Zap70, full-length Syk carries two N-terminal SH2 domains. These domains allow Syk to bind di-phosphorylated immunoreceptor tyrosine-based activation motifs (ITAMS) on the intercellular portion of a variety of receptors involved in immune regulation. Upon activation and recruitment to immunoreceptors, Syk phosphorylates a variety of cellular proteins including Linker for Activator of T-cells (LAT), B-cell Linker (BLNK), Vav, Bruton's Tyrosine Kinase, Gab, Bcap, SH2-domain containing Leukocyte Protein-76 (SLP-76) and Phospholipase Cγ.

In B-cells, Syk is essentially involved in B-cell Receptor (BCR) signal initiation, leading to development and survival of B lymphocytes in both bone marrow and periphery (Cheng et al. 1995, Nature, 378:3003; Turner et al. 1995 Nature, 378:298). It is activated by the Src-family kinase Lyn after Syk binds to doubly phosphorylated ITAMs on Igα/β chains on the BCR. The downstream effects of BCR engagement include Ca²⁺ flux, mitogen-activated protein (MAP) kinase activation & Akt activation. Signaling through the BCR is critical for development and survival of B lymphocytes in both bone marrow and periphery.

In mast cells and basophils, Syk is a critical component of FcεR1 signaling where downstream effects of activation include degranulation, release of cytokines such a tumor necrosis factor α and interleukin-6 and release of lipid mediators such as LTC4 (Costello et al. 1996 Oncogene, 13:2595). Similar Syk-dependent signaling is driven by IgG-antigen crosslinking via Fcγ receptors in macrophages, neutrophils & dendritic cells (Kiefer et al. 1998 Mol. Cell Biol., 18: 4209; Sedlik et al. 2003 J. Immun., 170:846). In macrophages, Syk activity is believed to regulate phagocytosis of opsonized foreign (and self) antigens via the FcγR, and Syk is important for antigen presentation from and maturation of dendritic cells. A role for Syk has been proposed for osteoclast maturation and in DAP12 receptor signaling in these cell types involved in bone metabolism. Reviews of these finding can be found in Expert Opin. Invest. Drugs, 2004, 13(7):743 and Expert Opin. Invest. Drugs, 2008, 17(5):641.

Therefore, Syk inhibition offers an opportunity to affect multiple cell types involved in inflammation, and it could be predicted to serve as therapy for autoimmune diseases including rheumatoid arthritis, asthma, systemic lupus erythematosus (SLE), and multiple sclerosis.

The Jak family kinases (Jak1, Jak2, Jak3 and Tyk2) are cytoplasmic tyrosine kinases that associate with membrane bound cytokine receptors. Cytokine binding to their receptor initiates Jak kinase activation via trans and autophosphorylation processes. The activated Jak kinases phosphorylate residues on the cytokine receptors creating phosphotyrosine binding sites for SH2 domain containing proteins such as Signal Transduction Activators of Transcript (STAT) factors and other signal regulators transduction such as SOCS proteins and SHIP phosphatases. Activation of STAT factors via this process leads to their dimerization, nuclear translocation and new mRNA transcription resulting in expression of immunocyte proliferation and survival factors as well as additional cytokines, chemokines and molecules that facilitate cellular trafficking (see Journal of Immunology, 2007, 178, p. 2623). Jak kinases transduce signals for many different cytokine families and hence potentially play roles in diseases with widely different pathologies including but not limited to the following examples. Both Jak1 and Jak3 control signaling of the so-called common gamma chain cytokines (IL2, IL4, IL7, IL9, IL15 and IL21), hence simultaneous inhibition of either Jak1 or Jak3 could be predicted to impact Th1 mediated diseases such as rheumatoid arthritis via blockade of IL2, IL7 and IL15 signaling. Th2 mediated diseases such as asthma or atopic dermatitis via IL4 and IL9 signaling blockade. Jak1 and Tyk2 mediate signaling of IL13 (see Int. Immunity, 2000, 12:1499). Hence, blockade of these may also be predicted to have a therapeutic effect in asthma. These two kinases are also thought to mediate Type I interferon signaling; their blockade could therefore be predicted to reduce the severity of systemic lupus erythematosus (SLE). Tyk2 and Jak2 mediate signaling of IL12 and IL23.

Jak2 is also activated in a wide variety of human cancers such as prostate, colon, ovarian and breast cancers, melanoma, leukemia and other haematopoietic malignancies. In addition, somatic point mutation of the Jak2 gene has been identified to be highly associated with classic myeloproliferative disorders (MPD) and infrequently in other myeloid disorders. Constitutive activation of Jak2 activity is also caused by chromosomal translocation in hematopoeitic malignancies. Accordingly, the identification of small-molecule compounds that inhibit, regulate and/or modulate the signal transduction of kinases, particularly Jak2, is desirable as a means to treat or prevent diseases and conditions associated with cancers.

All kinases bind a common molecule, ATP, and therefore have structurally similar binding pockets. Therefore, one of the challenges for any kinase inhibitor is that they are prone to inhibit more than one kinase due to the homology of the binding pocket. For example, staurosporine, a well characterized promiscuous kinase inhibitor, has been shown to inhibit at least 253 with a k_{d} of <3 μM kinases from the human kinome (see Nature Biotechnology, 2008, 26:127). Additionally, several marketed kinase inhibitors are known to inhibit more than one intended kinase, for example Iimatinib (Gleevec®) targets ABL, ARG, PDGFR-α/β and c-KIT kinases, sorafenib (Nexavar®) targets B-RAF, VEGFRs, PDGFR-α/β, FLT3 and c-KIT and sunitinib (Sutent®) targets VEGFR, PDGFR, CSF1R, FLT3 and c-KIT (Nature Reviews Drug Discovery 2011, 10:111).

Inhibition of certain kinases in the human kinome are known to have undesired effects when used as pharmaceutical treatment. For instance, a number of kinase targets have been implicated in playing a role in the cardiotoxicity profiles for kinase inhibitors that are currently on the market. These kinases can include, but not limited to, VEGFR2, PI3K, AKT, PDGFR-α/β, AMPK, GSK3, ERKs, CDK2, Aurora, PLK, JNK, CAMKII<PDK1, mTOR, LKB1, CAMKKβ, MEK1/2, PKA, PKCα, RAF1, B-RAF, EGFR, ERBB2, c-Kit, ABL, ARG, JAK2, FAK, DMPK, LTK, ROCK, LKB1, LDB3, PIM, GRK2, GRK5, ASK1, and PTEN (see Nature Reviews Drug Discovery, 2011, 10:111). One example from a marketed kinase inhibitor is that in clinical trials with sunitibnib, patients were found to be at increased risk for hypertension (see The Lancet, 2006, 368:1329 and J. Clin. Oncol., 2009, 27:3584). Subsequent research on the mechanism for the increased hypertension suggest that while PDGFR and VEGFR may be playing a role, off-target kinase inhibition, such as AMPK, may also be contributing to sunitinib's increased risk for hypertension (Curr. Hypertens. Rep., 2011, 13:436). Additionally, there is a patent application, US 2011/0212461, that has been filed that is a method for the prediction of cardiotoxicity based on the activity versus a list of kinases including CSF1R, KIT, FYN, PDGFR beta, FGR, LCK, Ephrin Receptor B2, FRK, ABL1, PDGFR1 alpha, HCK, ABL2, LYN, ZAK, YES1, MAP4K4, PKN1, BRAF, DDR2, MAP4K5 and STK24. Therefore, identification of kinase inhibitors with a selective profile Syk kinase are desirable. The compounds of this invention are selective for the inhibition of Syk over other kinases.

Many of the kinases, whether a receptor or non-receptor tyrosine kinase or a S/T kinase have been found to be involved in cellular signaling pathways involved in numerous pathogenic conditions, including immunomodulation, inflammation, or proliferative disorders such as cancer.

Many autoimmune diseases and disease associated with chronic inflammation, as well as acute responses, have been linked to excessive or unregulated production or activity of one or more cytokines.

The compounds of the invention are also useful in the treatment of rheumatoid arthritis, asthma, allergic asthma, osteoarthritis, juvenile arthritis, ankylosing spondylitis, an ocular condition, interstitial cystitis, a cancer, a solid tumor, a sarcoma, fibrosarcoma, osteoma, melanoma, retinoblastoma, a rhabdomyosarcoma, glioblastoma, neuroblastoma, teratocarcinoma, hypersensitivity reactions, hyperkinetic movement disorders, hypersensitivity pneumonitis, hypertension, hypokinetic movement disorders, aordic and peripheral aneuryisms, hypothalamic-pituitary-adrenal axis evaluation, aortic dissection, arterial hypertension, arteriosclerosis, arteriovenous fistula, ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, heart transplant rejection, hemachromatosis, hemodialysis, hemolytic uremic syndrome/thrombolytic thrombocytopenic purpura, hemorrhage, idiopathic pulmonary fibrosis, antibody mediated cytotoxicity, Asthenia, infantile spinal muscular atrophy, inflammation of the aorta, influenza A, ionizing radiation exposure, iridocyclitis/uveitis/optic neuritis, juvenile spinal muscular atrophy, lymphoma, myeloma, leukaemia, malignant ascites, hematopoietic cancers, a diabetic condition such as insulin-dependent diabetes mellitus glaucoma, diabetic retinopathy or microangiopathy, sickle cell anaemia, chronic inflammation, glomerulonephritis, graft rejection, Lyme disease, von Hippel Lindau disease, pemphigoid, Paget's disease, fibrosis, sarcoidosis, cirrhosis, thyroiditis, hyperviscosity syndrome, Osler-Weber-Rendu disease, chronic occlusive pulmonary disease, asthma or edema following burns, trauma, radiation, stroke, hypoxia, ischemia, ovarian hyperstimulation syndrome, post perfusion syndrome, post pump syndrome, post-MI cardiotomy syndrome, preeclampsia, menometrorrhagia, endometriosis, pulmonary hypertension, infantile hemangioma, or infection by Herpes simplex, Herpes Zoster, human immunodeficiency virus, parapoxvirus, protozoa or toxoplasmosis, progressive supranucleo palsy, primary pulmonary hypertension, radiation therapy, Raynaud's phenomenon, Raynaud's disease, Refsum's disease, regular narrow QRS tachycardia, renovascular hypertension, restrictive cardiomyopathy, sarcoma, senile chorea, senile dementia of Lewy body type, shock, skin allograft, skin changes syndrome, ocular or macular edema, ocular neovascular disease, scleritis, radial keratotomy, uveitis, vitritis, myopia, optic pits, chronic retinal detachment, post-laser treatment complications, conjunctivitis, Stargardt's disease, Eales disease, retinopathy, macular degeneration, restenosis, ischemia/reperfusion injury, ischemic stroke, vascular occlusion, carotid obstructive disease, ulcerative colitis, inflammatory bowel disease, diabetes, diabetes mellitus, insulin dependent diabetes mellitus, allergic diseases, dermatitis scleroderma, graft versus host disease, organ transplant rejection (including but not limited to bone marrow and solid organ rejection), acute or chronic immune disease associated with organ transplantation, sarcoidosis, disseminated intravascular coagulation, Kawasaki's disease, nephrotic syndrome, chronic fatigue syndrome, Wegener's granulomatosis, Henoch-Schoenlein purpurea, microscopic vasculitis of the kidneys, chronic active hepatitis, septic shock, toxic shock syndrome, sepsis syndrome, cachexia, infectious diseases, parasitic diseases, acquired immunodeficiency syndrome, acute transverse myelitis, Huntington's chorea, stroke, primary biliary cirrhosis, hemolytic anemia, malignancies, Addison's disease, idiopathic Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, peripheral vascular disorders, peritonitis, pernicious anemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis A, Hepatitis B, Hepatitis C, His bundle arrythmias, HIV infection/HIV neuropathy, common varied immunodeficiency (common variable hypogammaglobulinaemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, pneumocystis carinii pneumonia, pneumonia, connective tissue disease associated interstitial lung disease, mixed connective tissue disease, associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthritis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, acute and chronic pain (different forms of pain), Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, toxicity, transplants, and diseases involving inappropriate vascularization for example diabetic retinopathy, retinopathy of prematurity, choroidal neovascularization due to age-related macular degeneration, and infantile hemangiomas in human beings. In addition, such compounds may be useful in the treatment of disorders such as ascites, effusions, and exudates, including for example macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, preeclampsia, menometrorrhagia, cat scratch fever, rubeosis, neovascular glaucoma and retinopathies such as those associated with diabetic retinopathy, retinopathy of prematurity, or age-related macular degeneration. In addition, these compounds can be used as active agents against hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

Compounds of Formula (I) of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the compound of the present invention. The additional agent also can be an agent that imparts a beneficial attribute to the therapeutic composition e.g., an agent that affects the viscosity of the composition.

It should further be understood that the combinations which are to be included within this invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

Preferred combinations are non-steroidal anti-inflammatory drug(s) also referred to as NSAIDS which include drugs like ibuprofen. Other preferred combinations are corticosteroids including prednisolone; the well known side-effects of steroid use can be reduced or even eliminated by tapering the steroid dose required when treating patients in combination with the compounds of this invention. Non-limiting examples of therapeutic agents for rheumatoid arthritis with which a compound of Formula (I) of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, MMP-13 and PDGF. Compounds of the invention can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7 (U.S. Pat. No. 6,090,382, HUMIRA™), CA2 (REMICADE™), SIMPONI™ (golimumab), CIMZIA™, ACTEMRA™, CDP 571, and soluble p55 or p75 TNF receptors, derivatives, thereof, (p75TNFR1gG (ENBREL™) or p55TNFR1gG (Lenercept), and also TNFα converting enzyme (TACE) inhibitors; similarly IL-1 inhibitors (Interleukin-1-converting enzyme inhibitors, IL-1RA etc.) may be effective for the same reason. Other preferred combinations include Interleukin 11. Yet other preferred combinations are the other key players of the autoimmune response which may act parallel to, dependent on or in concert with IL-18 function; especially preferred are IL-12 antagonists including IL-12 antibodies or soluble IL-12 receptors, or IL-12 binding proteins. It has been shown that IL-12 and IL-18 have overlapping but distinct functions and a combination of antagonists to both may be most effective. Yet another preferred combination is non-depleting anti-CD4 inhibitors. Yet other preferred combinations include antagonists of the co-stimulatory pathway CD80 (B7.1) or CD86 (B7.2) including antibodies, soluble receptors or antagonistic ligands.

A compound of Formula (I) of the invention may also be combined with agents, such as methotrexate, 6-mercaptopurine, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g., NIK, IKK, JAK1, JAK2, JAK3, p38 or MAP kinase inhibitors), IL-1β converting enzyme inhibitors, T-cell signalling inhibitors such as kinase inhibitors, metalloproteinase inhibitors, sulfasalazine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™) and p55TNFRIgG (Lenercept), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, S1P1 agonists (such as FTY720), PKC family inhibitors (such as Ruboxistaurin or AEB-071) and Mesopram. Preferred combinations include methotrexate or leflunomide and in moderate or severe rheumatoid arthritis cases, cyclosporine and anti-TNF antibodies as noted above.

Non-limiting examples of therapeutic agents for inflammatory bowel disease with which a compound of Formula (I) of the invention can be combined include the following: budenoside; epidermal growth factor; corticosteroids; cyclosporin, sulfasalazine; amino salicylates; 6-mercaptopurine; azathioprine; metronidazole; lip oxygenase inhibitors; mesalamine; olsalazine; balsalazide; antioxidants; thromboxane inhibitors; IL-1 receptor antagonists; anti-IL-113 monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, IL-23, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signalling by proinflammatory cytokines such as TNFα or IL-1 (e.g. NIK, IKK, p38 or MAP kinase inhibitors); IL-1β converting enzyme inhibitors; TNFα converting enzyme inhibitors; T-cell signalling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-11, IL-13 and TGFβ). Preferred examples of therapeutic agents for Crohn's disease with which a compound of Formula (I) can be combined include the following: TNF antagonists, for example, anti-TNF antibodies, D2E7 (U.S. Pat. No. 6,090,382, HUMIRA™), CA2 (REMICADE™), CDP 571, TNFR-Ig constructs, (p75TNFRIgG (ENBREL™) and p55TNFRIgG (LENERCEPT™) inhibitors and PDE4 inhibitors. A compound of Formula (Ia), Formula (Ib), Formula (Ic), Formula (Id), Formula (Ie), Formula (If), Formula (Ig), Formula (Ih), Formula (Ii) or Formula (Ij) can be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors; 6-mercaptopurine; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-gamma.

Non-limiting examples of therapeutic agents for multiple sclerosis with which a compound of Formula (I) can be combined include the following: corticosteroids; prednisolone; methylprednisolone; azathioprine; cyclophosphamide; cyclosporine; methotrexate; 4-aminopyridine; tizanidine; interferon-β1α (AVONEX®; Biogen); interferon-β1b (BETASERON®; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE®; Teva Pharmaceutical Industries, Inc.); hyperbaric oxygen; intravenous immunoglobulin; cladribine; antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of Formula (Ia), Formula (Ib), Formula (Ic), Formula (Id), Formula (Ie), Formula (If), Formula (Ig), Formula (Ih), Formula (Ii) or Formula (Ij) can be combined with antibodies to cell surface molecules such as CD2, CD3, CD4, CD8, CD19, CD20, CD25, CD28, CD30, CD40, CD45, CD69, CD80, CD86, CD90 or their ligands. A compound of Formula (I) may also be combined with agents such as methotrexate, cyclosporine, FK506, rapamycin, mycophenolate mofetil, leflunomide, an S1P1 agonist, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g., NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

Preferred examples of therapeutic agents for multiple sclerosis in which a compound of Formula (I) can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

A compound of Formula (I) may also be combined with agents, such as alemtuzumab, dronabinol, daclizumab, mitoxantrone, xaliproden hydrochloride, fampridine, glatiramer acetate, natalizumab, sinnabidol, α-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL-4 agonists.

Non-limiting examples of therapeutic agents for ankylosing spondylitis with which a compound of Formula (I) can be combined include the following: ibuprofen, diclofenac, misoprostol, naproxen, meloxicam, indomethacin, diclofenac, celecoxib, rofecoxib, sulfasalazine, methotrexate, azathioprine, minocyclin, prednisone, and anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™)

Non-limiting examples of therapeutic agents for asthma with which a compound of Formula (I) can be combined include the following: albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin HCl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine HCl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone, anti-IL-13 antibody, and metaproterenol sulfate.

Non-limiting examples of therapeutic agents for COPD with which a compound of Formula (I) can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol HCl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, cilomilast and roflumilast.

Non-limiting examples of therapeutic agents for HCV with which a compound of Formula (I) (can be combined include the following: Interferon-alpha-2α, Interferon-alpha-2β, Interferon-alpha con1, Interferon-alpha-n1, pegylated interferon-alpha-2α, pegylated interferon-alpha-2β, ribavirin, peginterferon alfa-2b+ribavirin, ursodeoxycholic acid, glycyrrhizic acid, thymalfasin, Maxamine, VX-497 and any compounds that are used to treat HCV through intervention with the following targets: HCV polymerase, HCV protease, HCV helicase, and HCV IRES (internal ribosome entry site).

Non-limiting examples of therapeutic agents for Idiopathic Pulmonary Fibrosis with which a compound of Formula (I) (can be combined include the following: prednisone, azathioprine, albuterol, colchicine, albuterol sulfate, digoxin, gamma interferon, methylprednisolone sodium succinate, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone HCl, potassium chloride, triamcinolone acetonide, tacrolimus anhydrous, calcium, interferon-alpha, methotrexate, mycophenolate mofetil and interferon-γ-1β.

Non-limiting examples of therapeutic agents for myocardial infarction with which a compound of Formula (I) can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril hydrochloride/magnesium carbonate, bumetanide, alteplase, enalaprilat, amiodarone hydrochloride, tirofiban HCl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine HCl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, and cariporide.

Non-limiting examples of therapeutic agents for psoriasis with which a compound of Formula (I) can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine, ABT-874 and ustekinamab.

Non-limiting examples of therapeutic agents for psoriatic arthritis with which a compound of Formula (I) can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, D2E7 (U.S. Pat. No. 6,090,382, HUMIRA™), and efalizumab.

Non-limiting examples of therapeutic agents for restenosis with which a compound of Formula (I) can be combined include the following: sirolimus, paclitaxel, everolimus, tacrolimus, ABT-578, and acetaminophen.

Non-limiting examples of therapeutic agents for sciatica with which a compound of Formula (I) can be combined include the following: hydrocodone bitartrate/apap, rofecoxib, cyclobenzaprine HCl, methylprednisolone, naproxen, ibuprofen, oxycodone HCl/acetaminophen, celecoxib, valdecoxib, methylprednisolone acetate, prednisone, codeine phosphate/apap, tramadol HCl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone HCl, tizanidine HCl, diclofenac sodium/misoprostol, propoxyphene n-pap, asa/oxycod/oxycodone ter, ibuprofen/hydrocodone bit, tramadol HCl, etodolac, propoxyphene HCl, amitriptyline HCl, carisoprodol/codeine phos/asa, morphine sulfate, multivitamins, naproxen sodium, orphenadrine citrate, and temazepam.

Preferred examples of therapeutic agents for SLE (Lupus) with which a compound of Formula (I) can be combined include the following: NSAIDS, for example, diclofenac, naproxen, ibuprofen, piroxicam, indomethacin; COX2 inhibitors, for example, celecoxib, rofecoxib, valdecoxib; anti-malarials, for example, hydroxychloroquine; steroids, for example, prednisone, prednisolone, budenoside, dexamethasone; cytotoxics, for example, azathioprine, cyclophosphamide, mycophenolate mofetil, methotrexate; inhibitors of PDE4 or purine synthesis inhibitor, for example Cellcept®. A compound of Formula (I) may also be combined with agents such as sulfasalazine, 5-aminosalicylic acid, olsalazine, Imuran® and agents which interfere with synthesis, production or action of proinflammatory cytokines such as IL-1, for example, caspase inhibitors like IL-1β converting enzyme inhibitors and IL-1ra. A compound of Formula (I) may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of Formula (I) (can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of Formula (I) may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™).

In this invention, the following definitions are applicable:

A “therapeutically effective amount” is an amount of a compound of Formula (I) or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art.

“Pharmaceutically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g. (+) or (−)-tartaric acid or mixtures thereof), amino acids (e.g. (+) or (−)-amino acids or mixtures thereof), and the like. These salts can be prepared by methods known to those skilled in the art.

Certain compounds of Formula (I) which have acidic substituents may exist as salts with pharmaceutically acceptable bases. The present invention includes such salts. Examples of such salts include sodium salts, potassium salts, lysine salts and arginine salts. These salts may be prepared by methods known to those skilled in the art.

Certain compounds of Formula (I) and their salts may exist in more than one crystal form and the present invention includes each crystal form and mixtures thereof.

Certain compounds of Formula (I) and their salts may also exist in the form of solvates, for example hydrates, and the present invention includes each solvate and mixtures thereof.

Certain compounds of Formula (I) may contain one or more chiral centers, and exist in different optically active forms. When compounds of Formula (I) contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of Formula (I) contains more than one chiral center, it may exist in diastereoisomeric forms. The diastereoisomeric compounds may be separated by methods known to those skilled in the art, for example chromatography or crystallization and the individual enantiomers may be separated as described above. The present invention includes each diastereoisomer of compounds of Formula (I) (and mixtures thereof.

Certain compounds of Formula (I) may exist in different tautomeric forms or as different geometric isomers, and the present invention includes each tautomer and/or geometric isomer of compounds of Formula (I) and mixtures thereof.

Certain compounds of Formula (I) may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of Formula (I) and mixtures thereof.

Certain compounds of Formula (I) may exist in zwitterionic form and the present invention includes each zwitterionic form of compounds of Formula (I) (and mixtures thereof.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The pro-drug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial.

Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents wherein the free hydrogen is replaced by (C₁-C₄)alkyl, (C₁-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of Formula (Ia), Formula (Ib), Formula (Ic), Formula (Id), Formula (Ie), Formula (If), Formula (Ig), Formula (Ih), Formula (Ii) or Formula (Ij) wherein the free hydrogen of the hydroxyl substituent (e.g., R¹ contains hydroxyl) is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₁₂)alkoxycarbonyloxymethyl, N—(C₁-C₆)alkoxycarbonylamino-methyl, succinoyl, (C₁-C₆)alkanoyl, α-amino (C₁-C₄)alkanoyl, arylactyl and α-aminoacyl, or α-aminoacyl-α-aminoacyl wherein said a-aminoacyl moieties are independently any of the naturally occurring L-amino acids found in proteins, P(O)(OH)₂, —P(O)(O(C₁-C₆)alkyl)₂ or glycosyl (the radical resulting from detachment of the hydroxyl of the hemiacetal of a carbohydrate).

As used herein, the term “bridged (C₅-C₁₂) cycloalkyl group” means a saturated or unsaturated, bicyclic or polycyclic bridged hydrocarbon group having two or three C₃-C₁₀ cycloalkyl rings. Non bridged cycloalkyls are excluded. Bridged cyclic hydrocarbon may include, such as bicyclo[2.1.1]hexyl, bicyclo[2.2.1]heptyl, bicyclo[2.2.2]octyl, bicyclo[3.2.1]octyl, bicyclo[4.3.1]decyl, bicyclo[3.3.1]nonyl, bornyl, bornenyl, norbornyl, norbornenyl, 6,6-dimethylbicyclo[3.1.1]heptyl, tricyclobutyl, and adamantyl.

As used herein the term “bridged (C₂-C₁₀) heterocyclyl” means bicyclic or polycyclic aza-bridged hydrocarbon groups and may include azanorbornyl, quinuclidinyl, isoquinuclidinyl, tropanyl, azabicyclo[3.2.1]octanyl, azabicyclo[2.2.1]heptanyl, 2-azabicyclo[3.2.1]octanyl, azabicyclo[3.2.1]octanyl, azabicyclo[3.2.2]nonanyl, azabicyclo[3.3.0]nonanyl, and azabicyclo[3.3.1]nonanyl.

The term “heterocyclic,” “heterocyclyl” or “heterocyclylene,” as used herein, include non-aromatic, ring systems, including, but not limited to, monocyclic, bicyclic, tricyclic and spirocyclic rings, which can be completely saturated or which can contain one or more units of unsaturation, for the avoidance of doubt, the degree of unsaturation does not result in an aromatic ring system) and have 5 to 12 atoms including at least one heteroatom, such as nitrogen, oxygen, or sulfur. For purposes of exemplification, which should not be construed as limiting the scope of this invention, the following are examples of heterocyclic rings: azepinyl, azetidinyl, indolinyl, isoindolinyl, morpholinyl, piperazinyl, piperidinyl, pyrrolidinyl, quinucludinyl, thiomorpholinyl, tetrahydropyranyl, tetrahydrofuranyl, tetrahydroindolyl, thiomorpholinyl and tropanyl.

The term “heteroaryl” or “heteroarylene” as used herein, include aromatic ring systems, including, but not limited to, monocyclic, bicyclic and tricyclic rings, and have 5 to 12 atoms including at least one heteroatom, such as nitrogen, oxygen, or sulfur. For purposes of exemplification, which should not be construed as limiting the scope of this invention: azaindolyl, benzo(b)thienyl, benzimidazolyl, benzofuranyl, benzoxazolyl, benzothiazolyl, benzothiadiazolyl, benzoxadiazolyl, furanyl, imidazolyl, imidazopyridinyl, indolyl, indazolyl, isoxazolyl, isothiazolyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrazolyl, thiadiazolyl, thienyl, 6H-pyrrolo[2,3-e][1,2,4]triazolo[4,3-a]pyrazinyl, 6H-imidazo[1,5-a]pyrrolo[2,3-e]pyrazinyl, 1,6-dihydropyrazolo[3,4-d]pyrrolo[2,3-b]pyridine, 3H-3,4,6,8a-tetraaza-asindacenyl, 3H-imidazo[1,2-a]pyrrolo[2,3-e]pyrazinyl, pyrazolo[3,4-d]pyrrolo[2,3-b]pyridinyl, 1,6-dihydro-1,2,5,6-tetraza-as-indacenyl, 3H-3,4,8a-triaza-as-indacenyl, 6H-3-oxa-2,5,6-triaza-as-indacenyl, 3,6-dihydro-2,3,6-tetraaza-as-indacenyl, 1,6-dihydro-dipyrrolo[2,3-b;2′3′-d]pyridinyl, 6H-3-thia-2,5,6-triaza-as-indacenyl or 1,6-dihydroimidazo[4,5-d]pyrrolo[2,3-b]pyridine.

As used herein, “alkyl,” “alkylene,” or notations such as “(C₁-C₈)” include straight chained or branched hydrocarbons which are completely saturated. Examples of alkyls are methyl, ethyl, propyl, isopropyl, butyl, pentyl, hexyl and isomers thereof. As used herein, “alkenyl,” “alkenylene,” “alkynylene” and “alkynyl” means C₂-C₈ and includes straight chained or branched hydrocarbons which contain one or more units of unsaturation, one or more double bonds for alkenyl and one or more triple bonds for alkynyl.

As used herein, “aromatic” groups (or “aryl” or “arylene” groups) include aromatic carbocyclic ring systems (e.g. phenyl) and fused polycyclic aromatic ring systems (e.g. naphthyl, biphenyl and 1,2,3,4-tetrahydronaphthyl).

As used herein, “cycloalkyl” or “cycloalkylene” means C₃-C₁₂ monocyclic or multicyclic (e.g., bicyclic, tricyclic, spirocyclic, etc.) hydrocarbons that is completely saturated. Examples of a cycloalkyl group are cyclopropyl, cyclobutyl, cyclopentyl, and cyclohexyl.

As used herein, “cycloalkenyl” means C₃-C₁₂ monocyclic or multicyclic (e.g., bicyclic, tricyclic, spirocyclic, etc.) hydrocarbons that has one or more unsaturated bonds but does not amount to an aromatic group. Examples of a cycloalklenyl group are cyclopentenyl and cyclohexenyl.

As used herein, many moieties or substituents are termed as being either “substituted” or “optionally substituted”. When a moiety is modified by one of these terms, unless otherwise noted, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: (C₁-C₈)alkyl groups, (C₂-C₈)alkenyl groups, (C₂-C₈)alkynyl groups, (C₃-C₁₀)cyclo alkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, —NHC(O)H, —NHC(O) (C₁-C₈)alkyl groups, —NHC(O) (C₃-C₈)cycloalkyl groups, —N((C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, —N((C₁-C₈)alkyl)C(O)NH₂ groups, —NHC(O)N((C₁-C₈)alkyl)₂ groups, —N((C₁-C₈)alkyl)C(O)N((C₁-C₈)alkyl)₂ groups, —N((C₁-C₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cyclo alkyl groups, —S(O)₂NH₂ groups, —NHS(O)₂ (C₁-C₈)alkyl groups, —N((C₁-C₈)alkyl)S(O)₂(C₁-C₈)alkyl groups,—(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃),—(C₁-C₆) heterocycle (for example but not limited to pyrrolidine, tetrahydrofuran, pyran or morpholine),—(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

The term “kit” as used herein refers to a packaged product comprising components with which to administer a compound of Formula (I) of the invention for treatment of an autoimmune disorder. The kit preferably comprises a box or container that holds the components of the kit. The box or container is affixed with a label or a Food and Drug Administration approved protocol. The box or container holds components of the invention which are preferably contained within plastic, polyethylene, polypropylene, ethylene, or propylene vessels. The vessels can be capped-tubes or bottles. The kit can also include instructions for administering a compound of Formula (I).

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation. Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:SW) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (e.g., the concentration of the test compound which achieves a half-maximal inhibition of a given protein kinase activity). In some cases it is appropriate to determine the IC₅₀ in the presence of 3 to 5% serum albumin since such a determination approximates the binding effects of plasma protein on the compound. Such information can be used to more accurately determine useful doses in humans. Further, the most preferred compounds for systemic administration effectively inhibit protein kinase signaling in intact cells at levels that are safely achievable in plasma.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition (see e.g. Fingl et al., 1975, in The Pharmacological Basis of Therapeutics, Ch. 1 p. 1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition.

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the following Examples.

### a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

### b) Tablets

Tablets can be prepared, for example, from the following ingredients.

Parts by weight

| | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

### c) Enteric Coated Tablets

Tablets can be prepared by the method described in (b) above. The tablets can be enteric coated in a conventional manner using a solution of 20% cellulose acetate phthalate and 3% diethyl phthalate in ethanol:dichloromethane (1:1).

### d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

In the compositions of the present invention the active compound may, if desired, be associated with other compatible pharmacologically active ingredients. For example, the compounds of this invention can be administered in combination with another therapeutic agent that is known to treat a disease or condition described herein. For example, with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate. The additional pharmaceutical agents include, but are not limited to, anti-edemic steroids, NSAIDS, ras inhibitors, anti-TNF agents, anti-IL1 agents, antihistamines, PAF-antagonists, COX-1 inhibitors, COX-2 inhibitors, NO synthase inhibitors, Akt/PTB inhibitors, IGF-1R inhibitors, PKC inhibitors, PI3 kinase inhibitors, calcineurin inhibitors and immunosuppressants. The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deleterious effects of a hyperproliferative disorder, angiogenesis, vascular hyperpermeability or edema than the administration of either substance alone. In the treatment of malignant disorders combinations with antiproliferative or cytotoxic chemotherapies or radiation are included in the scope of the present invention.

The present invention also comprises the use of a compound of Formula (I) as a medicament.

A further aspect of the present invention provides the use of a compound of Formula (I) or a salt thereof in the manufacture of a medicament for treating vascular hyperpermeability, angiogenesis-dependent disorders, proliferative diseases and/or disorders of the immune system in mammals, particularly human beings.

The present invention also provides a method of treating vascular hyperpermeability, inappropriate neovascularization, proliferative diseases and/or disorders of the immune system which comprises the administration of a therapeutically effective amount of a compound of Formula (I) to a mammal, particularly a human being, in need thereof.

### ABBREVIATIONS

aa Amino acids
Ac Acyl or acetate
AcCl Acetyl chloride
Ac₂O Acetic anhydride
AcOH Glacial acetic acid
ATP Adenosine triphosphate
BINAP 2,2′-bis(diphenylphosphino)-1,1′-binaphthyl
Boc t-Butoxycarbonyl
Boc₂O Di-tert-butyl dicarbonate
BOP-Cl Bis(2-oxo-3-oxazolidinyl)phosphonic chloride
t-BuOH tert-Butanol
t-Bu₃P Tri-tert-butylphosphine
BSA Bovine serum albumin
CgPPh 1,3,5,7-Tetramethyl-6-phenyl-2,4,8-trioxa-6-phophaadamantane
d Doublet
dba Dibenzylideneacetone
DCE 1,2-Dichloroethane
DCM Dichloromethane (methylene chloride)
dd Doublet of doublets
DEAD Diethyl azodicarboxylate
DIAD Diisopropyl azodicarboxylate
DIEA N,N-Diisopropylethylamine
DME 1,2-Dimethoxyethane
DMF N,N-Dimethylformamide
DMSO Dimethyl sulfoxide
DNP-HSA Dinitrophenyl-human serum albumin
DTT Dithiothreitol
dppf 1,1′-Bis(diphenylphosphino)ferrocene
EDC 1-(3-Dimethylaminopropyl)-3-ethylcarbodiimide
Ee Enatiomeric excess
equiv Equivalent(s)
EtOAc Ethyl acetate
Et₂O Diethyl ether
EtOH Ethanol
FBS Fetal bovine serum
g Gram(s)
GM-CSF Granulocyte-macrophage colony-stimulating factor
GST Glutathione S-transferase
h Hour(s)
HATU O-(7-Azabenzotriazol-1-yl)-N,N,N′,N′-tetramethyluronium hexafluorophosphate
HEPES N-2-Hydroxyethylpiperazine-N′-2-ethanesulfonic acid
HOBt 1-Hydroxybenzotriazole
HPLC High-pressure liquid chromatography
HPMC Hydroxypropyl methylcellulose
IBCF Isobutyl chloroformate
i.d. Intradermal
IFA Incomplete Freunds Adjuvant
IPA Isopropyl alcohol
KOAc Potassium acetate
KOt-Bu Potassium tert-butoxide
LC/MS Liquid chromatography/mass spectrometry
m Multiplet
M Molar
MeCN Acetonitrile
MeI Iodomethane
MeOH Methyl alcohol
min Minute(s)
mmol Millimole
MS Mass spectrometry
n- Normal (nonbranched)
N Normal
NaOt-Bu Sodium tert-butoxide
NBS N-Bromosuccinimide
NCS N-Chlorosuccinimide
NH₄OAc Ammonium acetate
NMR Nuclear magnetic resonance
OD Optical density
or Optical rotation
OVA Ovalbumin
PBS Phosphate buffered saline
Pd₂dba₃ Tris(dibenzylideneacetone)dipalladium(0)
Pd(OAc)₂ Palladium(II) acetate
PdCl₂(dppf) (1,1′-bis(diphenylphosphino)ferrocene)dichloropalladium(II) complex with DCM
PdCl₂(MeCN)₂ Bis(acetonitrile)dichloropalladium(II)
pet ether Petroleum Ether
pH −log [H⁺]
Pd(PPh₃)₄ Tetrakis(triphenylphosphine)palladium(0)
Pd(PPh₃)₂Cl₂ Dichlorobis(triphenylphosphine)palladium(II)
PPh₃ Triphenylphosphine
ppm Parts per million
PrOH n-Propanol
psi Pounds per square inch
rcf Relative centrifugal force
RP-HPLC Reverse-phase high-pressure liquid chromatography
RPM Revolutions per minute
Rₜ Retention time
rt Room temperature
s Singlet
SFC Supercritical fluid chromatography
t Triplet
t- Tertiary
TEA Triethylamine
tert- Tertiary
tert-Butyl X-Phos 2-Di-tert-butylphosphino-2′,4′,6′-triisopropylbiphenyl
TFA Trifluoroacetic acid
TFAA Trifluoroacetic anhydride
THF Tetrahydrofuran
TLC Thin layer chromatography
TMS Trimethylsilyl
TMSI Trimethylsilyliodide
TsCl p-Toluenesulfonyl Chloride
USP United States Pharmacopeia
UV Ultraviolet
wt % Weight percent
Xantphos 4,5-Bis(diphenylphosphino)-9,9-dimethylxanthene
X-Phos 2-Dicyclohexylphosphino-2′,4′,6′-triisopropylbiphenyl

### Assays

In Vitro Syk Kinase Activity Measured by Time-Resolved Fluorescence Resonance Energy Transfer (trFRET)

Purified Syk catalytic domain (aa 356-635 with a C-terminal His-tag purified in-house by immobilized metal ion affinity chromatography; 0.14 nM final) was mixed with peptide substrate (biotin-TYR1, Sequence: Biotin-(Ahx)-GAEEEIYAAFFA-COOH, 0.2 μM final) at varying inhibitor concentrations in reaction buffer: 50 mM MOPSO pH 6.5, 10 mM MgCl₂, 2 mM MnCl₂, 2.5 mM DTT, 0.01% BSA, 0.1 mM Na₃VO₄ and 0.001 mM ATP. After about 60 min incubation at rt, the reaction was quenched by addition of EDTA (final concentration: 100 mM) and developed by addition of revelation reagents (final approximate concentrations: 30 mM HEPES pH 7.0, 0.06% BSA, 0.006% Tween-20, 0.24 M KF, 80 ng/mL PT66K (europium labeled anti-phosphotyrosine antibody cat #61T66KLB Cisbio, Bedford, Mass.) and 0.6 μg/mL SAXL (Phycolink streptavidin-allophycocyanin acceptor, cat #PJ25S, Prozyme, San Leandro, Calif.). The developed reaction was incubated in the dark either at about 4° C. for about 14 h or for about 60 min at rt, then read via a time-resolved fluorescence detector (Rubystar, BMG) using a 337 nm laser for excitation and monitoring emission wavelengths at 665 nm. Within the linear range of the assay, the observed signal at 665 nm was directly related to phosphorylated product and can be used to calculate the IC₅₀ values. For the purpose of the Tables and Examples below, the Syk IC₅₀ of each compound, which can be determined using the assay method described herein, is expressed as follows; A=a compound with a Syk IC₅₀ less than 0.01 μM, B=a compound with a Syk IC₅₀ within the range of 0.01 to 0.03 μM, C=a compound with a Syk IC₅₀ within the range of 0.03 to 0.1 μM and D=a compound with a Syk IC₅₀ greater than 0.1 μM.

Purchased Syk full-length enzyme (Millipore cat #14-314; more details in Table 1) was also used to evaluate enzyme potency. Additional kinase assays used to assess selectivity were performed using a similar protocol (see Table 1). Additional purified enzymes Jak1 (aa 845-1142; expressed in SF9 cells as a GST fusion and purified by glutathione affinity chromatography); Lck (aa 62-509; purified in-house by DEAE ion-exchange and ATP-sepharose affinity chromatography), and ITK (aa 354-620 with His tag, purified in-house by immobilized metal ion affinity and mono Q ion exchange chromatography) were expressed in SF9 cells. Other enzymes used are available from commercial sources. Enzymes were mixed with biotinylated substrates at varying concentrations of inhibitor in different reaction buffers (see Table 1). After about 60 min incubation at rt, the reaction was quenched by addition of EDTA and developed by addition of revelation reagents (final approximate concentrations: 30 mM HEPES pH 7.0, 0.06% BSA, 0.006% Tween-20, 0.24 M KF, varying amounts of donor europium labeled antibodies and acceptor streptavidin labeled allophycocyanin (SAXL)). The developed reactions were incubated in the dark at about 4° C. for about 14 h or for about 60 min at rt, then read in a time-resolved fluorescence detector (Rubystar, BMG Labtech) as described above.

In Vitro Screening for Other Kinase Activity Measured by Time-Resolved Fluorescence Resonance Energy Transfer (trFRET)

TR-FRET kinome profiling is performed as follows. The concentrations of individual components in each individual kinase reaction are based on optimized conditions; these include kinases (2-10 nM; sources-Life Technologies, Millipore, CarnaBio, in-house), Oregon Green- or Alexa-647-labeled fluorescent probes (2×K_{d} of probe to the particular kinase; range of 6.25-200 nM; in-house/Life Technologies), Terbium-anti-HIS or -anti-GST antibody (2 nM; Life Technologies). The trRFET reaction buffer consists of 20 mM HEPES pH7.4/10 mM MgCL2/0.0075% Triton X-100/100 uM Sodium Orthovanadate/1 mM dTT. Typically, compounds are tested across the range of 0.0001-10 μM in a 10-fold dilution dose response. The final [DMSO] of the reaction does not exceed 2%. Reactions are carried out in a 20 μl volume in PerkinElmer Proxiplate-plus 384-well white assay plates (cat. no. 6008289), by combining kinase/probe/antibody/test compound, mixing, and finally allowing the reaction to come to equilibrium (incubation of 2.5 hours). Fluorescence is then measured on a PerkinElmer Envision plate reader (Emission+Excitation parameters: Oregon Green/Terbium-340+520/495 nm; Alexa-647/Europium-340+665/615 nm). All results calculations are performed on the ratio of the two emission values (probe/antibody). Successful competition by the test compound vs. the fluorescent probe for the ATP binding site of each kinase is quantified by calculating the IC₅₀ value based on a 4 parameter logistic curve fit. The Ki value is then calculated using the Cheng-Prusoff equation (Kᵢ=(IC₅₀)/(1+[probe]/K_{d}). Finally, the TR-FRET kinome profile of a given compound is visualized in a heatmap which is generated in Spotfire after converting Kᵢ to pKᵢ values (pKᵢ=6-LOG [Kᵢ]). Accuracy and precision are assessed weekly by testing the control Staurosporine against each kinase in every experiment. MSSR analysis is being implemented to track assay performance.

TABLE 1
Specific conditions (per 40 μL enzyme reaction or 20 μl TR-FRET reaction)
for the various enzymes are detailed below:

| | | Substrate | | | | | | | |
|---|---|---|---|---|---|---|---|---|---|
| | | (TR- | | Enzyme | Substrate | ATP | DMSO | Reaction | |
| | | FRET | Assay | Conc. | Conc. | Conc. | Conc. | Time | Detection |
| Enzyme | Construct | probe) | Buffer | (nM) | (nM) | (mM) | (%) | (min) | condition |
| cMET | Life | (MP3) | TR-FRET | 4 | 12.5 | 0 | 2 | 150 | Fluorescence |
| | Technologies | | competition | | | | | | 340 + 520 ex/ |
| | cat# | | binding | | | | | | 495 em |
| | PV3143 | | buffer | | | | | | |
| CSF1R | Life | (PR1) | TR-FRET | 5 | 100 | 0 | 2 | 150 | Fluorescence |
| | Technologies | | competition | | | | | | 340 + 520 ex/ |
| | cat# | | binding | | | | | | 495 em |
| | PV3249 | | buffer | | | | | | |
| FGFR1 | Life | (PR1) | TR-FRET | 5 | 200 | 0 | 2 | 150 | Fluorescence |
| | Technologies | | competition | | | | | | 340 + 520 ex/ |
| | cat# | | binding | | | | | | 495 em |
| | PV3146 | | buffer | | | | | | |
| Flt1 | Life | (PR1) | TR-FRET | 2.5 | 100 | 0 | 2 | 150 | Fluorescence |
| | Technologies | | competition | | | | | | 340 + 520 ex/ |
| | cat# | | binding | | | | | | 495 em |
| | PV3666 | | buffer | | | | | | |
| Jak2 | Millipore | Biotin- | MOPSO | 2.5 | 2000 | 0.001 | 5 | 60 | 8 ng/well |
| | cat# 14-640 | TYR1 | | ng/well | | | | | PT66K, |
| | | | | | | | | | 0.078 μg/well |
| | | | | | | | | | SAXL |
| Kdr | Life | (PR1) | TR-FRET | 5 | 50 | 0 | 2 | 150 | Fluorescence |
| | Technologies | | competition | | | | | | 340 + 520 ex/ |
| | cat# | | binding | | | | | | 495 em |
| | PV3660 | | buffer | | | | | | |
| MAP4K2 | Life | (PR2) | TR-FRET | 7.5 | 100 | 0 | 2 | 150 | Fluorescence |
| | Technologies | | competition | | | | | | 340 + 520 ex/ |
| | cat# | | binding | | | | | | 495 em |
| | PV4211 | | buffer | | | | | | |
| MST1 | Life | (PR1) | TR-FRET | 2.5 | 50 | 0 | 2 | 150 | Fluorescence |
| | Technologies | | competition | | | | | | 340 + 520 ex/ |
| | cat# | | binding | | | | | | 495 em |
| | PV3854 | | buffer | | | | | | |
| PAK4KD | Life | (PR2) | TR-FRET | 2.5 | 100 | 0 | 2 | 150 | Fluorescence |
| | Technologies | | competition | | | | | | 340 + 520 ex/ |
| | cat# | | binding | | | | | | 495 em |
| | PV4212 | | buffer | | | | | | |
| RET | Life | (PR3) | TR-FRET | 2.5 | 100 | 0 | 2 | 150 | Fluorescence |
| | Technologies | | competition | | | | | | 340 + 520 ex/ |
| | cat# | | binding | | | | | | 495 em |
| | PV3819 | | buffer | | | | | | |
| RSK2 | Life | (PR2) | TR-FRET | 5 | 12.5 | 0 | 2 | 150 | Fluorescence |
| | Technologies | | competition | | | | | | 340 + 520 ex/ |
| | cat# | | binding | | | | | | 495 em |
| | PV3323 | | buffer | | | | | | |
| TBK1 | Life | (PR1) | TR-FRET | 5 | 6.25 | 0 | 2 | 150 | Fluorescence |
| | Technologies | | competition | | | | | | 340 + 520 ex/ |
| | cat# | | binding | | | | | | 495 em |
| | PV3504 | | buffer | | | | | | |

### Reaction Buffers:

MOPSO buffer contains: 50 mM MOPSO pH 6.5, 10 mM MgCl₂, 2 mM MnCl₂, 2.5 mM DTT, 0.01% BSA, and 0.1 mM Na₃VO₄.

HEPES buffer contains: 50 mM HEPES pH 7.1, 2.5 mM DTT, 10 mM MgCl₂, 2 mM MnCl₂, 0.01% BSA, and 0.1 mM Na₃VO₄.

TR-FRET competition binding buffer consists of 20 mM HEPES pH7.4/10 mM MgCL2/0.0075% Triton X-100/100 uM Sodium Orthovanadate/1 mM dTT.

MOPS buffer contains: 20 mM MOPS pH 7.2, 10 mM MgCl₂, 5 mM EGTA, 5 mM Beta-phosphoglycerol, 1 mM Na₃VO₄, 0.01% Triton-X-100 and 1 mM DTT.

### Substrates:

Biotin-TYR1-peptide sequence: Biotin-(Ahx)-GAEEEIYAAFFA-COOH

Biotin-TYR2-peptide sequence: Biotin-(Ahx)-AEEEYFFLFA-amide

KinEASE S1 peptides were purchased from Cisbio (cat #62STOPEB, Bedford, Mass.)

TR-FRET probes: PR1 and PR2 are Staurosporine-based; PR3 and MP3 are based on proprietary Abbott/AbbVie kinase inhibitors.

### Detection Reagents:

PT66K was purchased from Cisbio (cat #61T66KLB, Bedford, Mass.)

EuSTK was purchased from Cisbio (cat #62STOPEB, Bedford, Mass.)

SAXL was purchased from Prozyme (cat #PJ25S, San Leandro, Calif.)

Some examples of activities expressed as IC₅₀s (M) for the compounds of the invention are shown in Table 2.

TABLE 2

| Example | #A.1.4 | #E.1.1 | #F.1.10 | #F.1.14 | #F.1.15 | #6 | #7 |
|---|---|---|---|---|---|---|---|
| cMET | >10 | 0.8 | 2.6 | 2.3 | 1.7 | 7.7 | 9.1 |
| CSF1R | >10 | >10 | 4.0 | >10 | 7.1 | >10 | >10 |
| FGFR1 | 6.9 | 3.9 | >10 | 9.5 | 1.6 | 4.6 | 3.0 |
| Flt1 | >10 | 7.1 | 5.7 | >10 | >10 | >10 | >10 |
| JAK2 | 0.9 | 0.09 | 0.4 | 4.8 | 0.2 | 1.7 | 0.4 |
| JAK3 | 1.6 | 0.3 | 0.8 | 4.9 | 0.4 | 3.9 | 0.7 |
| Kdr | 6.3 | 3.4 | 1.1 | 5.5 | 1.7 | 1.5 | 3.5 |
| MAP4K2 | 5.3 | 2.7 | 1.8 | >10 | 0.2 | 0.02 | 1.9 |
| MST1 | 1.4 | 0.8 | 7.3 | >10 | >10 | 0.1 | 0.9 |
| PAK4KD | 2.1 | 0.2 | 0.09 | 2.5 | 0.5 | 0.6 | 1.3 |
| RET | 0.8 | 0.2 | 0.2 | 3.7 | 0.13 | 1.2 | 0.7 |
| RSK2 | 1.3 | 0.7 | 0.1 | 0.5 | 0.4 | 0.2 | 0.4 |
| TBK1 | >10 | >10 | >10 | >10 | >10 | 2.7 | >10 |

### Antigen-Induced Degranulation of RBL-2H3 Cells:

RBL-2H3 cells were maintained in T75 flasks at about 37° C. and 5% CO₂, and passaged every 3-4 days. To harvest cells, 20 mL of PBS was used to rinse the flask once, and then 3 mL of Trypsin-EDTA was added and incubated at about 37° C. for about 2 min. Cells were transferred to a tube with 20 mL medium, spun down at 1000 RPM at rt for about 5 min and resuspended at 1×10⁶ cells/mL. Cells were sensitized by adding DNP-specific mouse IgE (Sigma #D8406) to a final concentration of 0.1 μg/mL. 50 μL of cells were added to each well of a 96 well flat bottom plate (50×10³ cells/well) and incubated overnight at about 37° C. in 5% CO₂. The next day, compounds were prepared in 100% DMSO at 10 mM. Each compound was then serially diluted 1:4 six times in 100% DMSO. Each compound dilution was then diluted 1:20 and then 1:25, both dilutions in Tyrode's buffer (HBSS with Ca⁺⁺ and Mg⁺⁺ (Gibco #14025)+20 mM Hepes (Gibco #15630)+0.2 mg/mL BSA (Sigma #8527,)+5.6 mM glucose (Sigma G8270). Media was aspirated from the cell plates and the cells were rinsed twice with 100 μL of Tyrode's buffer (prewarmed to about 37° C.). 50 μL of compounds diluted in Tyrode's buffer were added to each well and the plates were incubated for about 15 min at about 37° C. in 5% CO₂. 50 μL of 0.2 μg/mL DNP-HSA (Bioresearch Technologies, Inc. #D-5059-100) in Tyrode's buffer was then added to each well and the plates were incubated for about 30 min at about 37° C. in 5% CO₂. The final concentration of the various components in the incubation mix are 0.002-10 μM compounds, 0.1% DMSO, and 0.1 μg/mL DNP-HSA. As one control, Tyrode's buffer with DNP-HSA was added to a set of wells containing 0.2% DMSO without compounds to determine maximum stimulated release. As a second control, Tyrode's buffer without DNP-HSA was added to a set of wells containing 0.2% DMSO without compounds to determine unstimulated release. At the end of the 30 min incubation, 100 μL of freshly prepared 1 mM 4-methylumbelliferyl N-acetyl-13-D-glucosaminide (MUG; Sigma #M2133) was then added to each well and the plates are incubated for about 45 min at about 37° C. in 5% CO₂. The plates were then read on an EnVision plate reader with excitation=355 nm, emission=460 nm. Within the linear range of the assay, the observed signal at 460 nm was directly related to MUG reaction product production and can be used to calculate the IC₅₀ values.

### Ramos Calcium Flux Assay

### Materials:

Ramos cells (ATCC # CRL-1596) were maintained at 37° C. and 5% CO₂ in T150 flasks. Culture medium: RPMI medium (Invitrogen #21870-075) supplemented with 10% heat-inactivated FBS (Invitrogen #10438-026) and 1% Pen/Strep (Invitrogen #15140-122). Assay Buffer: HBSS (Invitrogen: #14025-092) with 40 mM Hepes (Invitrogen #15630-080), 0.1% Bovine Serum Albumin (BSA) (Sigma #A8577), 2.5 mM Probenecid (Invitrogen #P36400) and 10 mM Glucose (Sigma #G-7528). Other materials used in this assay: DMSO (Sigma D8418), 96-well dilution plates (polypropylene) (Corning #3365), 96-well assay plates (Corning #3603), FLIPR Calcium 5 Assay Bulk Kit (Molecular Devices #R8186), and Donkey anti-human IgM Affinity Purified Fab2 (Jackson ImmunoResearch Laboratories #709-006-073), FLIPR TETRA machine (Molecular Devices).

### Methods:

Cells were seeded at 5×10⁵ cells/mL in culture medium 16-18 hours before assay. On the day of the assay, cells were centrifuged at 1000 rpm for 5 min, resuspended in culture medium, and counted. An appropriate volume of cell suspension was set at a concentration of 2×10⁶ cells/mL in regular culture medium and plated in assay plates at 2×10⁵ cells/well (100 μL/well). A stock solution of Calcium 5 dye was prepared by adding 10 mL of assay buffer per vial of dye from the bulk kit. A 2× dye solution was prepared by adding 1 mL of dye stock solution to 9 mL of assay buffer, added to assay plates (100 μL/well) and incubated for 1 h at 37° C. and 5% CO₂ DMSO compound stocks were prepared by dissolving and serially diluting test compounds or controls in 100% DMSO. Immediately before compound testing, DMSO compound stocks were diluted 1:33 in assay buffer to make a 6× compound stock (2% DMSO). Using the FLIPR Tetra machine, 6× compound stock was transferred to the assay plate (50 μL/well, 0.33% final DMSO) and potential calcium flux was monitored for 3.5 min following compound addition (Excitation wavelength: 470/495 nm; Emission wavelength: 515/575 nm; 1^{st} read interval: 1 second, number of reads: 60; number of reads before dispensing: 10; 2^{nd} interval read: 6 seconds, # of reads: 30). Compounds were incubated for 30 min at rt. A 6× stimulus solution was made fresh before addition to cells by diluting 1.3 mg/mL anti-IgM antibody stock solution to 60 μg/mL in assay buffer. After compound incubation, the FLIPR Tetra machine transferred 6× stimulus solution to cells (50 μL/well, anti-IgM antibody final 10 μg/mL) and calcium flux was monitored for 3.5 min following antibody addition (Excitation wavelength: 470/495 nm; Emission wave length: 515/575 nm; 1^{st} read interval: 2 second, number of reads: 60; number of reads before dispensing: 10; 2^{nd} interval read: 6 seconds, number of reads: 80). The IC₅₀ values for compounds tested were then calculated based on percent of inhibition of anti-IGM antibody induced calcium flux.

Acute in vivo measurement of Fcγ receptor signaling inhibition of the compounds is measured using the:

Acute in vivo measurement of Fcγ receptor signaling inhibition of the compounds is measured using the:

### Reverse Passive Arthus Model

On day 0 OVA was made up at a concentration of 17 mg/mL, in PBS by rocking gently until a solution was formed. 2% Evans Blue solution (Sigma Aldrich, cat# E2129) was then added to double the volume for a final concentration of 8.5 mg/mL of OVA and 1% Evans Blue dye. Anti-OVA antibody (Abazyme), stock concentration 10 mg/mL, was thawed and a 4 mg/mL solution was made with PBS. Compounds were made up in 0.5% HPMC with 0.02% Tween 80, and vortexed for about 15 seconds followed by homogenization for a minimum of about 2 min at 28,000 RPM until there was a fine particulate suspension with no clumps of compound. Rats were weighed and dosed with compound at a pre-determined time based on compound Tₘₐₓ determined in pharmacokinetic studies. Animals were then placed under general anesthesia with a 5% isoflourane and oxygen mixture and shaved. Using a 0.5 cc insulin syringe three sites were injected i.d., 2 site with 100 μL of 4.0 mg/mL of anti-OVA antibody, and 1 site with 100 μL of sterile PBS. Immediately following i.d. injections, animals were injected with 200 μL of the OVA (10 mg/kg)/1% Evans Blue mixture, i.v., using a 0.5 cc insulin syringe. About 4 h post injection animals were euthanized, bled via cardiac puncture and blood was put into plasma separation tubes. Blood samples were stored on ice until centrifugation (within about 2 h of collection). Each injection site was removed with a disposable biopsy punch (Acuderm Acu-Punch Disposable 12 mm), cut into four pieces and placed in a pre-labeled 2 mL eppendorf tube. One mL of DMF (99%) was added to each biopsy tube and they were placed in a heat block at about 50° C. for about 24 h. After incubation, 100 μL of each sample was transferred to a 96 well flat bottom plate in duplicate and read at 620 nm on a plate reader using the Softmax software. Background was removed by subtracting the OD from the PBS injected site from the OD of the anti-OVA injected site for each individual animal. Plasma samples were spun down in a microcentrifuge (Eppendorf 5415R) for about 5 min at 16.1 rcf. 20 μL of plasma was placed in a round bottom 96 well plate for drug level measurements and tubes were stored at about −80° C. until evaluation.

### Collagen Induced Arthritis (CIA)

Type II Collagen (CII), derived from bovine nasal septum (Elastin Products, cat# CN276) was solubilized in 0.01M AcOH (150 μL AcOH USP grade, J.T. Baker, order#9522-03, and 250 mL Milli Q Water) to give a concentration of 4 mg/mL. The vial was covered with aluminum foil and placed on a rocker at about 4° C. overnight. The collagen stock solution was diluted 1:1 with incomplete Freunds adjuvant (IFA) (Difco labs, cat#263910) and an emulsion was made in glass Hamilton luer lock syringes (SGE Syringe Perfection VWR cat#007230), to a final concentration of 2 mg/mL. Female Lewis rats, approximately 8 weeks of age, (Charles River Laboratories) were anesthetized in an anesthesia chamber using isoflurane (5%) and oxygen. Anesthesia was maintained using a nose cone during the injections. Rats were shaved at the base of the tail and 600 μg of collagen was delivered in three 100 μL i.d. injections on the rump of the rat (n=9 per group). A negative control group was immunized with a 1:1 emulsion of 0.01 M AcOH and IFA (n=6). Animals were boosted on day 6 of the study in the same manner as the immunization. Compound dosing began 11 days after the initial immunization when first signs of disease were observed. Compounds were formulated in an inert vehicle such as 0.5% HPMC (Sigma, cat# H3785)/0.02% Tween 80 (Sigma, cat#4780) in water) and dosed orally once or twice a day for at least 7 days. Baseline body weight and paw volume was taken on day 8, prior to disease onset, using a water displacement pleythsmograph (Vgo Basile North America Inc. PA 19473, Model#7140). Animals were placed under general anesthesia with a 5% isoflourane and oxygen mixture and both hind paws were dipped into the plethysmograph and the paw volume was recorded. The rats were scored 3 times a week from day 11-17 after immunization. On day 17 after immunization, rats were sacrificed by CO₂ inhalation and were exsanguinated by cardiac puncture under isoflurane anesthesia, and the hind paws were collected to assess the impact on bone erosion using micro-CT scans (SCANCO Medical, Southeastern, PA, Model# μCT 40) at a voxel size of 18 μm, a threshold of 400, sigma-gauss 0.8, support-gauss 1.0. Bone volume and density was determined for a 360 μm (200 slice) vertical section encompassing the tarsal section of the paw. The 360 μm section was analyzed from the base of the metatarsals to the top of the tibia, with the lower reference point fixed at the tibiotalar junction. Three rats from each of the dosing groups were tail nicked on Day 17 at various time points and blood was collected in plasma separation tubes. Plasma samples were spun down in a microcentrifuge (Eppendorf 5415R) for about 5 min at 16.1 ref. 20 μL of plasma was placed in a round bottom 96 well plate for drug level measurements and tubes were stored at about −80° C. until evaluation.

The teachings of all references, including journal articles, patents and published patent applications, are incorporated herein by reference in their entirety.

The following examples are for illustrative purposes and are not to be construed as limiting the scope of the present invention.

### General Synthetic Schemes

Compounds of the invention may be prepared using the synthetic transformations illustrated in Schemes I-IV. Starting materials are commercially available, may be prepared by the procedures described herein, by literature procedures, or by procedures that would be well known to one skilled in the art of organic chemistry. It should be of note that alternate protecting groups may be employed where appropriate and the examples included within are not limiting (see Greene, T. W. and Wus, P. G. M. Protecting Groups in Organic Synthesis, 3^{rd} Edition, 1999, Wiley-Interscience).

The General Procedure used is designated as its bolded capital letter (e.g. A for General Procedure A).

Methods for preparing 3-substituted (5-tosyl-5H-pyrrolo[2,3-b]pyrazin-2-yl)methanamine compounds of the invention are illustrated in Scheme I. (5-Tosyl-SH-pyrrolo[2,3-b]pyrazin-2-yl)methanamine hydrochloride 1 is prepared according to US 2011/0311474, Example #5, Steps A through C. The benzylic amine is then protected with a Boc protecting group (Scheme I, step a) and the tosyl is removed using methods known to one skilled in the art (for example as described in Preparation #2, steps A and B) to give tert-butyl ((5H-pyrrolo[2,3-b]pyrazin-2-yl)methyl)carbamate 2. Compound 2 can then be halogenated at the 7-position (Scheme I, step b), for example with NCS, NBS or NIS, to give a tert-butyl ((7-halo-5H-pyrrolo[2,3-b]pyrazin-2-yl)methyl)carbamates 3 using methods described in Preparation #2, Step C or Preparation #3, Step A. Subsequent reactions using a base and a tosylating agent (Scheme I, step c) gives the corresponding tert-butyl ((7-halo-5-tosyl-5H-pyrrolo[2,3-b]pyrazin-2-yl)methyl)carbamates 4 (see for example, Preparation #1, Step D or Preparation #3, Step B). At this point, the tert-butyl ((7-halo-5-tosyl-5H-pyrrolo[2,3-b]pyrazin-2-yl)methyl)carbamates 4 can either have the Boc protecting group removed (Scheme I, step d) using methods known to one skilled in the art to give (7-halo-5-tosyl-5H-pyrrolo[2,3-b]pyrazin-2-yl)methanamines 5 that can be used directly as starting materials for Scheme II, where R₁ is a halogen atom, or the halogen atom can be used as a functional group for a Suzuki reaction with alkylboronic acids, alkylboronates and alkylboroxines (Scheme I, step e) to give tert-butyl ((7-alkyl-5-tosyl-5H-pyrrolo[2,3-b]pyrazin-2-yl)methyl)carbamates 6 (for example, see Preparation #2, Step E). The Boc protecting group is then removed (Scheme I, step f) from the tert-butyl ((7-alkyl-5-tosyl-5H-pyrrolo[2,3-b]pyrazin-2-yl)methyl)carbamates 6 using methods known to one skilled in the art to give (7-halo-5-tosyl-5H-pyrrolo[2,3-b]pyrazin-2-yl)methanamines 7 that can be used directly as starting materials for Scheme II, where R₁ is an alkyl group.

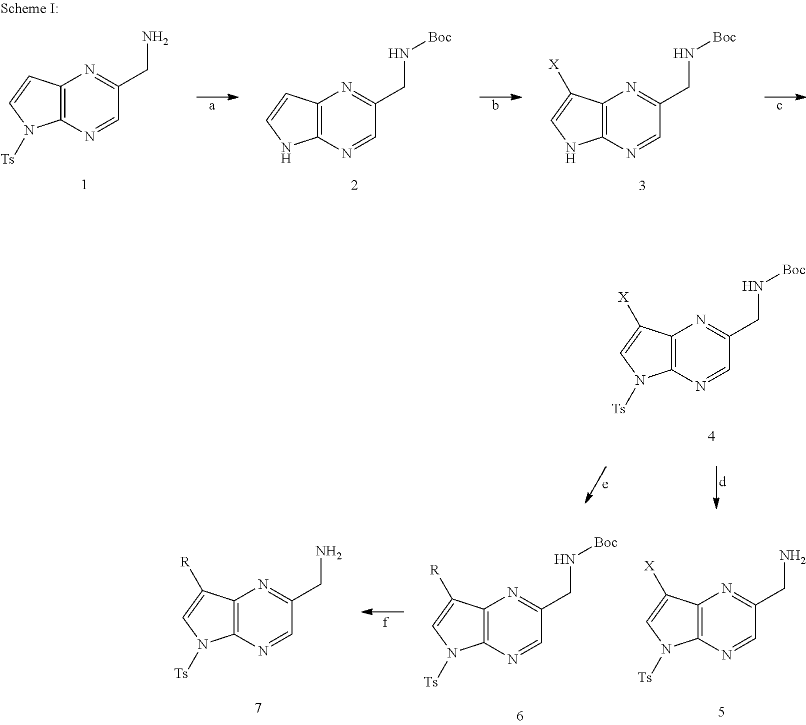

Methods for preparing (3R,5R)-1-acetyl-5-(3-halo-8-chloro-6-tosyl-6H-imidazo[1,5-a]pyrrolo[2,3-e]pyrazin-1-yl)pyrrolidin-3-yl acetate compounds 14 of the invention are illustrated in Scheme II. The (5-tosyl-SH-pyrrolo[2,3-b]pyrazin-2-yl)methanamines 8 can be optionally substituted at the 7-position, represented by R₁, with a halogen or alkyl substituent. In Scheme II, the (5-tosyl-SH-pyrrolo[2,3-b]pyrazin-2-yl)methanamines 8 can be reacted with (2R,4R)-1-(tert-butoxycarbonyl)-4-hydroxypyrrolidine-2-carboxylic acid 9 to give (2R,4R)-tert-butyl 4-hydroxy-2-((5-tosyl-5H-pyrrolo[2,3-b]pyrazin-2-yl)methyl)carbamoyl)pyrrolidine-1-carboxylates 10 (Scheme II, step a) using standard methods known to one skilled in the art (for example, see Preparation #1, Step B). The (2R,4R)-1-(tert-butoxycarbonyl)-4-hydroxypyrrolidine-2-carboxylic acid 9 used in Scheme II, step a is prepared from commercially available (2R,4R)-1-tert-butyl 2-methyl 4-hydroxypyrrolidine-1,2-dicarboxylate (Oakwood) using the conditions described in Preparation #1, Step A. The Boc group is removed from the give (2R,4R)-tert-butyl 4-hydroxy-2-(((5-tosyl-5H-pyrrolo[2,3-b]pyrazin-2-yl)methyl)carbamoyl)pyrrolidine-1-carboxylates 10 (Scheme II, step b) using methods known to one skilled in the art (for example, see Preparation #1, Step C to give (2R,4R)-4-hydroxy-N-((5-tosyl-5H-pyrrolo[2,3-b]pyrazin-2-yl)methyl)pyrrolidine-2-carboxamides 11 that are subsequently acylated at both the oxygen and nitrogen (Scheme II, step c) using methods known to one skilled in the art (for example, see Preparation #1, Step D) to give (3R,5R)-1-acetyl-5-(((5-tosyl-5H-pyrrolo[2,3-b]pyrazin-2-yl)methyl)carbamoyl)pyrrolidin-3-yl acetates 12. Conversion of the (3R,5R)-1-acetyl-5-(((5-tosyl-5H-pyrrolo[2,3-b]pyrazin-2-yl)methyl)carbamoyl)pyrrolidin-3-yl acetates 12 directly to the (3R,5R)-1-acetyl-5-(6-tosyl-6H-imidazo[1,5-a]pyrrolo[2,3-e]pyrazin-1-yl)pyrrolidin-3-yl acetates 13 (Scheme II, step d) can be carried out under anhydrous acidic conditions (for example, see Preparation #1, Step E). Alternate methods for this cyclicazation may be employed as described in WO2011/068899. The resulting (3R,5R)-1-acetyl-5-(6-tosyl-6H-imidazo[1,5-a]pyrrolo[2,3-e]pyrazin-1-yl)pyrrolidin-3-yl acetates 13 are then halogenated at the 3-position using a halogenating agent, such as NCS, NBS or NIS, (Scheme II, step e) to give (3R,5R)-1-acetyl-5-(3-halo-6-tosyl-6H-imidazo[1,5-a]pyrrolo[2,3-e]pyrazin-1-yl)pyrrolidin-3-yl acetates 14 (for example, see Preparation #1, Step F) that can be used directly as starting materials for Scheme III where R₁ is optionally either a halogen or alkyl substituent.

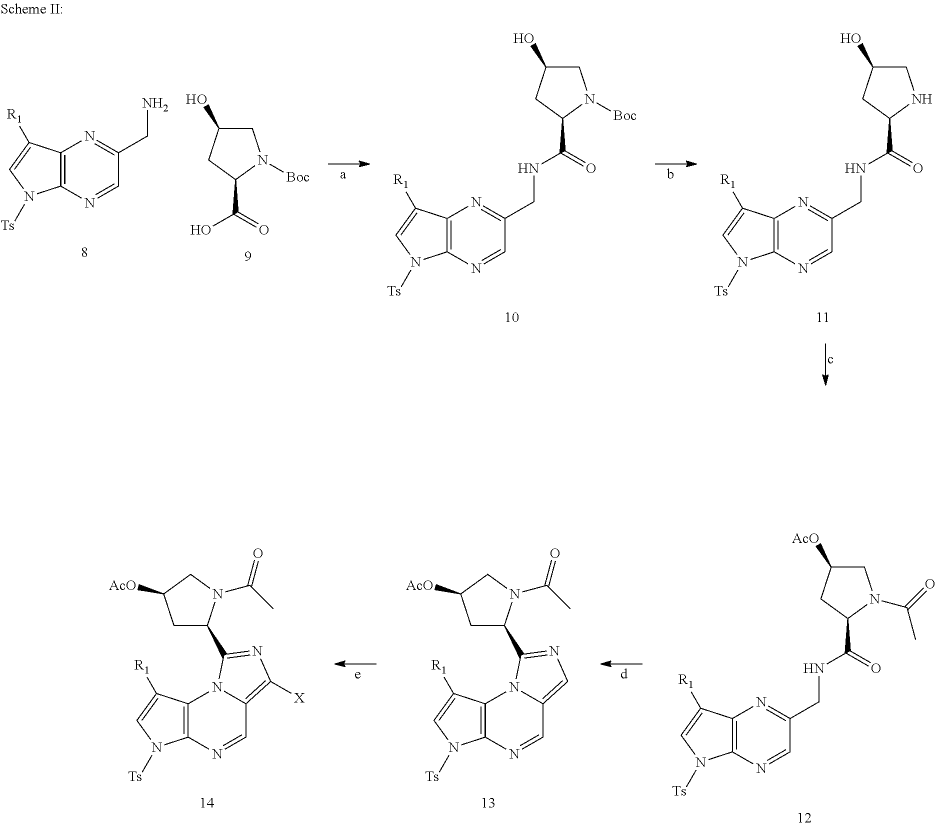

Methods for preparing targeted 1-((2R,4R)-4-hydroxy-2-(3-aryl/heteroaryl-6H-imidazo[1,5-a]pyrrolo[2,3-e]pyrazin-1-yl)pyrrolidin-1-yl)ethanone compounds 17 of the invention are described in Scheme III. 1-((2R,4R)-4-hydroxy-2-(3-aryl/heteroaryl-6H-imidazo[1,5-a]pyrrolo[2,3-e]pyrazin-1-yl)pyrrolidin-1-yl)ethanone compounds 17 can be optionally substituted at the 8-position, represented by R₁, with a halogen or alkyl substituent and are substituted at the 3-position, represented by R₂, with an optionally substituted aryl or heteroaryl substituent. The (3R,5R)-1-acetyl-5-(3-halo-6-tosyl-6H-imidazo[1,5-a]pyrrolo[2,3-e]pyrazin-1-yl)pyrrolidin-3-yl acetates 14 from Scheme II can either be reacted under Suzuki conditions with aryl or heteroaryl boronic acids or boronates (Scheme III, step a) to give (3R,5R)-1-acetyl-5-(3-aryl/heteroaryl-6-tosyl-6H-imidazo[1,5-a]pyrrolo[2,3-e]pyrazin-1-yl)pyrrolidin-3-yl acetates 15 (for example, see General Procedure A). Subsequent deprotection of the both the N-tosyl and O-acyl groups using methods known to one skilled in the art (Scheme III, step b) give the targeted 1-((2R,4R)-4-hydroxy-2-(3-aryl/heteroaryl-6H-imidazo[1,5-a]pyrrolo[2,3-e]pyrazin-1-yl)pyrrolidin-1-yl)ethanones 17 (for example, see General Procedure D). Additionally, these two reactions can be carried out in one pot using methods described in General Procedure F. Alternatively, the sequence can be reversed where the (3R,5R)-1-acetyl-5-(3-halo-6-tosyl-6H-imidazo[1,5-a]pyrrolo[2,3-e]pyrazin-1-yl)pyrrolidin-3-yl acetates 14 are deprotected (Scheme III, step c) to give 1-((2R,4R)-2-(3-halo-6H-imidazo[1,5-a]pyrrolo[2,3-e]pyrazin-1-yl)-4-hydroxypyrrolidin-1-yl)ethanones 16 which are then subjected to Suzuki reaction conditions (Scheme III, step d) with aryl or heteroaryl boronic acids or boronates using the methods described above to give the targeted 1-((2R,4R)-4-hydroxy-2-(3-aryl/heteroaryl-6H-imidazo[1,5-a]pyrrolo[2,3-e]pyrazin-1-yl)pyrrolidin-1-yl)ethanones 17. The aryl and heteroaryl boronic acids or boronates are either commercially available or prepared using methods known to one skilled in the art (for example, see Merino et al Angew. Chem Int. Ed. 2010, 49, 7164 or General Procedure B).

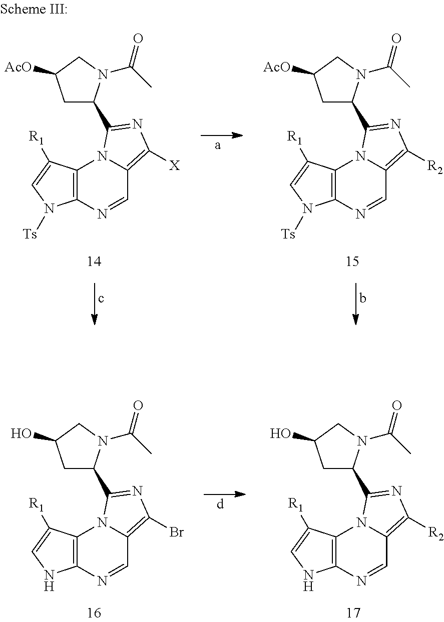

Additional functional groups can be introduced to the R₂ substituent either before the Suzuki reaction, for example at the aryl or heteroarylhalide stage, or aryl or heteroaryl boronic acid or bornate stage, or after the Suzuki reaction. Some examples include Buchwald reaction of a aryl halide with an analine (see General Procedure C), formation of an amide from an amine and a carboxylic acid (see General Procedure E) and formation of ethers either through alkylation chemistry (see General Procedure G) or Mitsunobu reactions (see General Procedure H). See Larock, R. C. Comprehensive Organic transformations L A Guide to Functional Group Preparations, 2^{nd} Edition, 1999, Wiliey-VCH for additional examples and procedures.

Some additional modification to the ((2R,4R)-4-hydroxy-2-(3-aryl/heteroaryl-6H-imidazo[1,5-a]pyrrolo[2,3-e]pyrazin-1-yl)pyrrolidin-1-yl)ethanones 17 includes removing the N-acyl group (Scheme IV, step a) using methods described in Preparation #5 to give a (3R,5R)-5-(3-chloro-6H-imidazo[1,5-a]pyrrolo[2,3-e]pyrazin-1-yl)pyrrolidin-3-ols 18 and subsequent formation of an amide bond using conditions described in General Procedure E (Scheme IV, step b) to give the targeted ((2R,4R)-4-hydroxy-2-(3-aryl/heteroaryl-6H-imidazo[1,5-a]pyrrolo[2,3-e]pyrazin-1-yl)pyrrolidin-1-yl)alkanones 19 where R₃ is an optionally substituted alkyl group. Alternatively, R₃ can be a NH₂ functionality (Scheme IV, step b) by reacting the (3R,5R)-5-(3-chloro-6H-imidazo[1,5-a]pyrrolo[2,3-e]pyrazin-1-yl)pyrrolidin-3-ols 18 with isocyanatotrimethylsilane as described in Example #6, Step C.

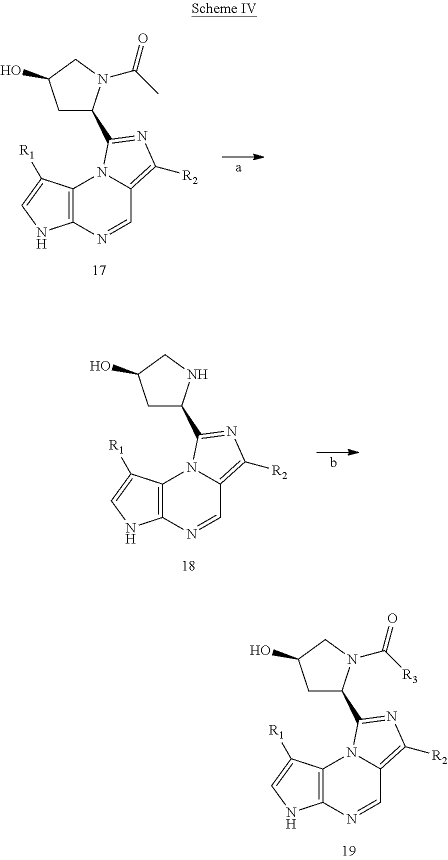

### GENERAL PROCEDURES AND EXAMPLES

The general synthetic schemes that were utilized to construct the majority of compounds disclosed in this application are described below in Schemes 1-8. These schemes are provided for illustrative purposes only and are not to be construed as limiting the scope of the invention.

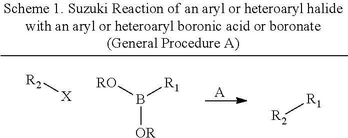

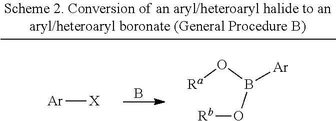

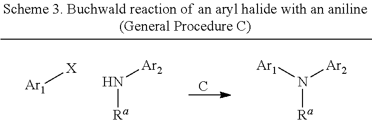

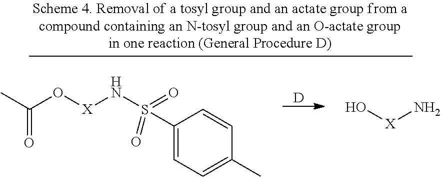

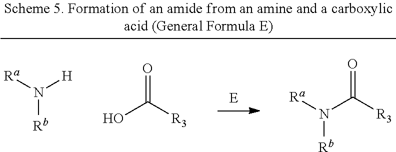

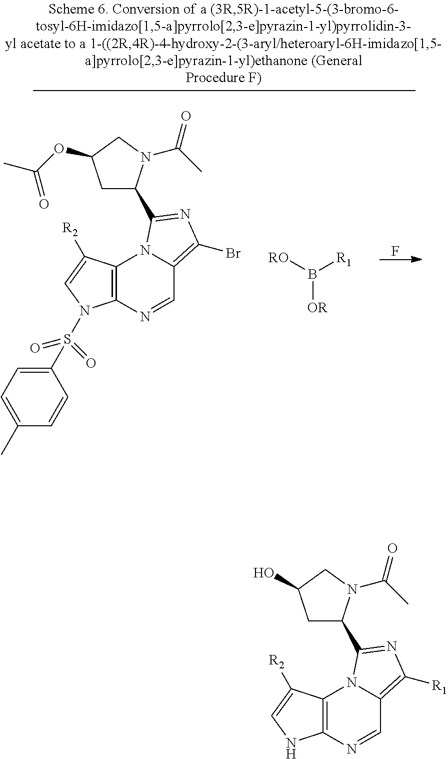

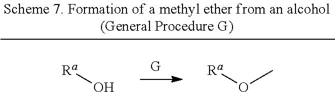

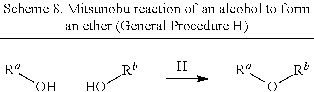

### List of General Procedures

General Procedure A Suzuki Reaction of an aryl or heteroaryl halide with an aryl or heteroaryl boronic acid or boronate
General Procedure B Conversion of an aryl/heteroaryl halide to an aryl/heteroaryl boronate
General Procedure C Buchwald reaction of an aryl halide with an aniline
General Procedure D Removal of a tosyl group and an acetate group from a compound containing an N-tosyl Group and an O-actuate group in one reaction
General Procedure E Formation of an amide from an amine and a carboxylic acid
General Procedure F Conversion of a (3R,5R)-1-acetyl-5-(3-bromo-6-tosyl-6H-imidazo[1,5-a]pyrrolo[2,3-e]pyrazin-1-yl)pyrrolidin-3-yl acetate to a 1-((2R,4R)-4-hydroxy-2-(3-aryl/heteroaryl-6H-imidazo[1,5-a]pyrrolo[2,3-e]pyrazin-1-yl)pyrrolidin-1-yl)ethanone
General Procedure G Formation of a methyl ether from an alcohol
General Procedure H Mitsunobu reaction of an alcohol to form an ether

The following examples are ordered according to the final general procedure used in their preparation. The synthetic routes to any novel intermediates are detailed by sequentially listing the general procedure (letter codes) in parentheses after their name with additional reactants or reagents as appropriate. A worked example of this protocol is given below from Example #5. In Example #5 1-((2R,4R)-4-hydroxy-2-(3-(4-((S)-2-methylmorpholino)phenyl)-6H-imidazo[1,5-a]pyrrolo[2,3-e]pyrazin-1-yl)pyrrolidin-1-yl)ethanone and 1-((2R,4R)-4-hydroxy-2-(3-(4-((R)-2-methylmorpholino)phenyl)-6H-imidazo[1,5-a]pyrrolo[2,3-e]pyrazin-1-yl)pyrrolidin-1-yl)ethanone was prepared from (3R,5R)-1-acetyl-5-(3-bromo-6-tosyl-6H-imidazo[1,5-a]pyrrolo[2,3-e]pyrazin-1-yl)pyrrolidin-3-yl acetate (0.525 g, 0.937 mmol, Preparation #1), 2-methyl-4-(4-(4,4,5,5-tetramethyl-1,3,2-dioxaborolan-2-yl)phenyl)morpholine (0.312 g, 1.030 mmol, prepared using C from 1,4-dibromobenzene and 2-methylmorpholine and B using 4,4,5,5-tetramethyl-[1,3,2]dioxaborolane) as represented in Scheme A.

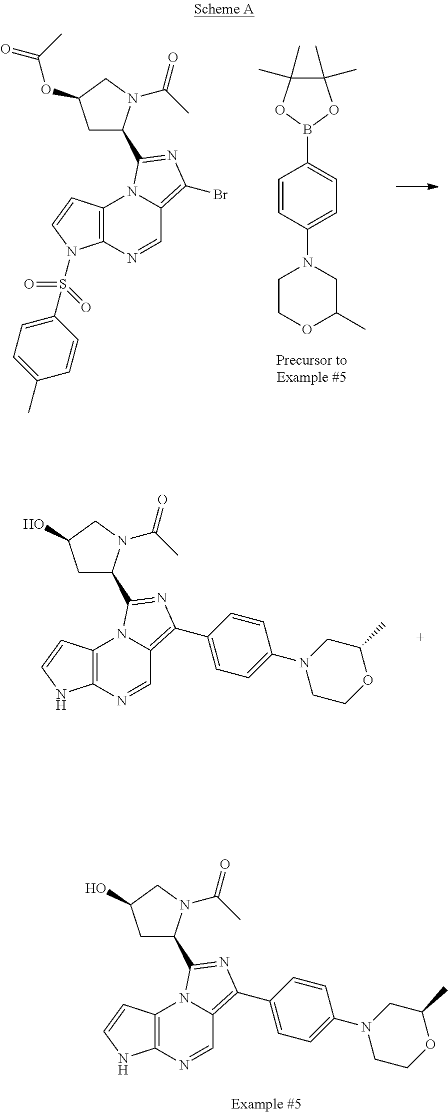

The precursor to Example #5, 2-methyl-4-(4-(4,4,5,5-tetramethyl-1,3,2-dioxaborolan-2-yl)phenyl)morpholine, was prepared as shown in Scheme B. 1,4-dibromobenzene and 2-methylmorpholine are reacted following conditions given in General Procedure C to give 4-(4-bromophenyl)-2-methylmorpholine. The intermediate is then reacted with 4,4,5,5-tetramethyl-1,3,2-dioxaborolane using the conditions described in General Procedure B to give the precursor to Example #5. The reaction sequence detailed above is translated in the preparations and examples section to “prepared using C from 1,4-dibromobenzene and 2-methylmorpholine and B using 4,4,5,5-tetramethyl-[1,3,2]dioxaborolane.”

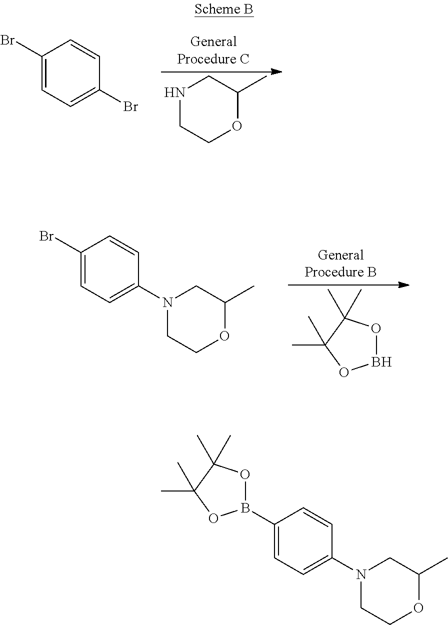

### Analytical Methods

Analytical data is included within the procedures below, in the illustrations of the general procedures, or in the tables of examples. Unless otherwise stated, all ¹H NMR data were collected on a Varian Mercury Plus 400 MHz, a Varian Inova 600 MHz instrument, a Varian Inova 500 MHz, a Bruker AVIII 400, or a Bruker AVIII 300 MHz instrument and chemical shifts are quoted in parts per million (ppm). LC/MS and HPLC data is referenced to the table of LC/MS and HPLC conditions using the lower case method letter provided in Table 3.

TABLE 3
LC/MS and HPLC methods

| Method | Conditions |
|---|---|
| a | LC/MS: The gradient was 5 to 60% B in 1.5 min |
| | then 60 to 95% B to 2.5 min with a hold at 95% |
| | B for 1.2 min (1.3 mL/min flow rate). Mobile |
| | phase A was 10 mM NH₄OAc, mobile phase |
| | B was HPLC grade MeCN. The column |
| | used for the chromatography was a |
| | 4.6 × 50 mm MAC-MOD Halo C18 column |
| | (2.7 μm particles). Detection methods were |
| | diode array (DAD) and evaporative light |
| | scattering (ELSD) detection as well as |
| | positive/negative electrospray ionization. |
| b | LC/MS: The gradient was 5 to 60% B in 1.5 min |
| | then 60 to 95% B to 2.5 min with a hold at 95% |
| | B for 1.2 min (1.3 mL/min flow rate). Mobile |
| | phase A was 10 mM NH₄OAc, mobile phase |
| | B was HPLC grade MeCN. The column |
| | used for the chromatography was a |
| | 4.6 × 50 mm MAC-MOD Halo C8 column |
| | (2.7 μm particles). Detection methods were |
| | diode array (DAD) and evaporative light |
| | scattering (ELSD) detection as well as |
| | positive/negative electrospray ionization. |
| c | LC/MS: The gradient was 5 to 60% B in 0.75 min |
| | then 60 to 95% B to 1.15 min with a hold at 95% |
| | B for 0.75 min (1.3 mL/min flow rate). Mobile |
| | phase A was 10 mM NH₄OAc, mobile phase |
| | B was HPLC grade MeCN. The column |
| | used for the chromatography was a |
| | 4.6 × 50 mm MAC-MOD Halo C8 column |
| | (2.7 μm particles). Detection methods were |
| | diode array (DAD) and evaporative light |
| | scattering (ELSD) detection as well as |
| | positive/negative electrospray ionization. |
| d | LC/MS: The gradient was 5% B in 0.2 min |
| | then 5 to 95% B in 1.2 min with a hold at 95% |
| | B for 1.5 min (2.0 mL/min flow rate). Mobile |
| | phase A was 10 mM NH₄HCO₃, |
| | mobile phase B was HPLC grade MeCN. |
| | The column used for the chromatography was a |
| | 4.6 × 50 mm XBridge18 (3.5 μm particles) |
| | and the temperature was 50° C. Detection |
| | methods are diode array (DAD) and evaporative |
| | light scattering (ELSD) detection as well as |
| | positive/negative electrospray ionization. |
| e | LC/MS: The gradient was 5 to 60% B in 0.60 min |
| | then 60-95% B to 1.00 min with a hold at 95% |
| | B for 0.30 min (1.3 mL/min flow rate). Mobile |
| | phase A was 10 mM NH₄OAc, mobile phase |
| | B was HPLC grade MeCN. The column used |
| | for the chromatography was a 2.1 × 50 mm |
| | ACE Excel 2 UHPLC C18 column (2.0 μm |
| | particles). Detection methods were diode |
| | array (DAD) and evaporative light scattering |
| | (ELSD) detection as well as positive/negative |
| | electrospray ionization. |
| f | Preparatory SFC: The gradient was 5 to 50% MeOH |
| | (0.1% DEA): CO₂ for 10 min |
| | (3.0 mL/min flow rate). The column used for |
| | the chromatography was a ChiralCel OJ-H. |
| | Detection methods are diode array (DAD) and |
| | evaporative light scattering (ELSD) detection as well |
| | as positive/ negative electrospray ionization. |
| g | SFC/MS: The gradient was 5 to 55% B co-solvent 7 |
| | min then hold at 55% for 1 min (4 mL/min flow rate, |
| | 100 bar, 37° C.). Solvent A was SFC grade |
| | CO₂. Co-solvent B was HPLC grade methanol |
| | with 0.1% diethylamine added. The column used |
| | for the chromatography was a 4.6 × 250 mm |
| | Daicel OJH column (5 μm particles). |
| | Detection methods were diode array (DAD) |
| | and evaporative light scattering (ELSD) |
| | detection as well as positive/negative electrospray |
| | ionization. |
| h | Preparatory Chiral Chromatography: The gradient was |
| | 0.5% B for 3.1 min then 0.5 to 10% B in 3.8 min |
| | (20 mL/min flow rate). Mobile phase A was HPLC |
| | grade heptane with 0.1% diethylamine. Mobile Phase |
| | B was EtOH. The column used for the chromatography |
| | was a 20 × 250 mm Daicel (5 μm particles). |
| | Detection methods were diode array (DAD) and |
| | evaporative light scattering (ELSD) detection |
| i | LC/MS: The gradient was 5 to 95% B in 1.3 min |
| | with a hold at 95% B for 1.5 min (1.8 mL/min |
| | flow rate). Mobile phase A was 10 mM NH₄OAc, |
| | mobile phase B was HPLC grade MeCN. The |
| | column used for the chromatography was a |
| | 4.6 × 50 mm XBridgec18 column (3.5 μm |
| | particles) with a column temperature of 50° C. |
| | Detection methods were diode array (DAD) and |
| | evaporative light scattering (ELSD) detection as well |
| | as positive/negative electrospray ionization. |
| j | Prepratory HPLC: The gradient was 10 to 50% |
| | B in 8.7 min with a hold at 50% B for 3.2 min |
| | (30 mL/min flow rate). Mobile phase A was 10 mM |
| | NH₄OAc, mobile phase B was HPLC grade MeCN. |
| | The column used for the chromatography was a 19 × 250 mm |
| | XBridge Prep C18 column (10 μm particles). Detection |
| | methods were diode array (DAD) and evaporative |
| | light scattering (ELSD) detection |

### General Purification Methods

For the general procedures, the final compounds may be purified by any technique or combination of techniques known to one skilled in the art. Some examples that are not limiting include column chromatography with a solid phase (i.e. silica gel, alumina, etc.) and a solvent (or combination of solvents) that elutes the desired compounds (i.e. hexanes, heptane, EtOAc, DCM, MeOH, EtOH, MeCN, water, etc.); preparatory TLC with a solid phase (i.e. silica gel, alumina etc.) and a solvent (or combination of solvents) that elutes the desired compounds (i.e. hexanes, heptane, EtOAc, DCM, MeOH, EtOH, MeCN, water, etc.); reverse phase HPLC (see Table 3 for some non-limiting conditions); recrystalization from an appropriate solvent (i.e. MeOH, EtOH, IPA, EtOAc, toluene, etc.) or combination of solvents (i.e. EtOAc/heptane, EtOAc/MeOH, etc.); chiral LC with a solid phase and an appropriate solvent (i.e. EtOH/heptane, MeOH/heptane, IPA/heptane, etc. with or without a modifier such as diethylamine, TFA, etc.) to elute the desired compound; chiral SFC with a solid phase and CO₂ with an appropriate modifier (i.e. MeOH, EtOH, IPA with or without additional modifier such as diethylamine, TFA, etc.); precipitation from a combination of solvents (i.e. DMF/water, DMSO/DCM, EtOAc/heptane, etc.); trituration with an appropriate solvent (i.e. EtOAc, DCM, MeCN, MeOH, EtOH, IPA, PrOH, etc.); extractions by dissolving a compound in a liquid and washing with an appropriately immiscible liquid (i.e. DCM/water, EtOAc/water, DCM/saturated NaHCO₃, EtOAc/saturated NaHCO₃, DCM/10% aqueous HCl, EtOAc/10% aqueous HCl, etc.); distillation (i.e. simple, fractional, Kugelrohr, etc.); gas chromatography using an appropriate temperature, carrier gas and flow rate; sublimation at an appropriate temperature and pressure; filtration through a media (i.e. Florosil®, alumina, Celite®, silica gel, etc.) with a solvent (i.e. heptane, hexanes, EtOAc, DCM, MeOH, etc.) or combination of solvents; salt formation with solid support (resin based, i.e. ion exchange) or without. Some descriptions of these techniques can be found in the following references, Gordon, A. J. and Ford, R. A. The Chemist's Companion, 1972; Palleros, D. R. Experimental Organic Chemistry, 2000; Still, W. C., Kahn, M. and Mitra, A. J. Org. Chem., 1978, 43:2923; Yan, B. Analysis and Purification Methods in Combinatorial Chemistry 2003; Harwood, L. M., Moody, C. J. and Percy, J. M. Experimental Organic Chemistry: Standard and Microscale, 2^{nd} Edition, 1999; Stichlmair, J. G. and Fair, J. R. Distillation; Principles and Practices, 1998; Beesley T. E. and Scott, R. P. W. Chiral Chromatography, 1999; Landgrebe, J. A. Theory and Practice in the Organic Laboratory, 4^{th} Edition, 1993; Skoog, D. A. and Leary, J. J. Principles of Instrumental Analysis, 4^{th} Edition, 1992; Subramanian, G. Chiral Separation Techniques, 3^{rd} Edition, 2007; Kazakevich, Y. and Lobrutto, R. HPLC for Pharmaceutical Scientists, 2007. Final or intermediate compounds prepared via any of the following General Procedures can be optionally purified using one or more of the purification methods described above.

### Preparations and Examples

The general synthetic methods used in each General Procedure follow and include an illustration of a compound that was synthesized using the designated General Procedure. None of the specific conditions and reagents noted herein are to be construed as limiting the scope of the invention and are provided for illustrative purposes only. All starting materials are commercially available from Sigma-Aldrich (including Fluka and Discovery CPR) unless otherwise noted after the chemical name. Reagent/reactant names given are as named on the commercial bottle or as generated by IUPAC conventions, CambridgeSoft® ChemDraw Ultra 9.0.7, CambridgeSoft® Chemistry E-Notebook 9.0.127, or AutoNom 2000. Compounds designated as salts (e.g. hydrochloride, acetate) may contain more than one molar equivalent of the salt.

### Preparation #1. Preparation of (3R,5R)-1-Acetyl-5-(3-bromo-6-tosyl-6H-imidazo[1,5-a]pyrrolo[2,3-e]pyrazin-1-yl)pyrrolidin-3-yl acetate

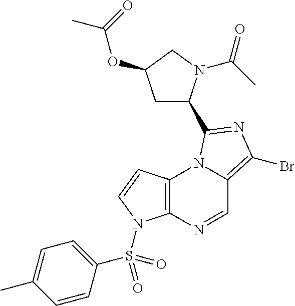 [SMILES: CC(=O)O[C@@H]1C[C@H](c2nc(Br)c3cnc4c(ccn4S(=O)(=O)c4ccc(C)cc4)n23)N(C(C)=O)C1]

### Step A: (2R,4R)-1-(tert-Butoxycarbonyl)-4-hydroxypyrrolidine-2-carboxylic acid

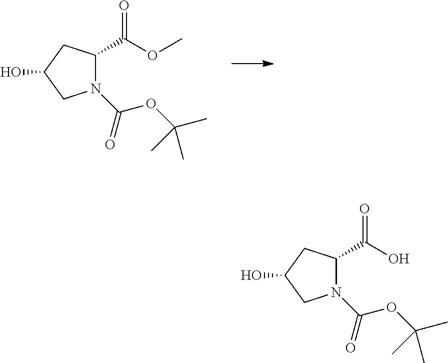 [SMILES: COC(=O)[C@H]1C[C@@H](O)CN1C(=O)OC(C)(C)C ; CC(C)(C)OC(=O)N1C[C@H](O)C[C@@H]1C(=O)O]

To an aqueous solution of NaOH (2 N, 1100 mL, 2275 mmol) was added to methyl (2R,4R)-1-tert-butyl 2-methyl 4-hydroxypyrrolidine-1,2-dicarboxylate (279 g, 1140 mmol, Oakwood) followed by MeOH (3300 mL). The mixture was stirred at about 60° C. for about 2 h and the mixture was partially concentrated in vacuo removing ˜3 L of solvent. The remaining solution was cooled in an ice bath and concentrated HCl was used to adjust the pH to between 2 and 3 while maintaining the temperature at about 15° C. The resulting precipitate was collected by filtration and rinsed with DCM (2×100 mL). The combined filtrate was separated and the organic phase was dried over MgSO₄, filtered and concentrated in vacuo. This material was combined with the solids collected in the first filtration to provide (2R,4R)-1-(tert-butoxycarbonyl)-4-hydroxypyrrolidine-2-carboxylic acid: LC/MS (Table 3, Method b) Rₜ=1.10 min.; MS m/z: 232 (M+H)⁺.

### Step B: (2R,4R)-1-(tert-Butoxycarbonyl)-4-hydroxypyrrolidine-2-carboxylic acid

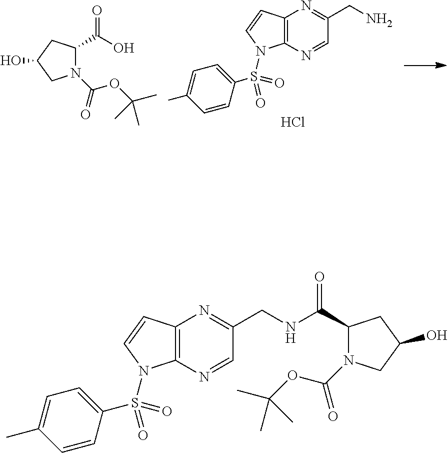

To a mixture of (5-tosyl-5H-pyrrolo[2,3-b]pyrazin-2-yl)methanamine hydrochloride (192 g, 567 mmol, US 2011/0311474, Example #5, Step C) and (2R,4R)-1-(tert-butoxycarbonyl)-4-hydroxypyrrolidine-2-carboxylic acid (131 g, 567 mmol) and HATU (226 g, 595 mmol) in DCM (5100 mL) at rt was added DIEA (208 mL, 1190 mmol) drop wise over about 2 h. After about 18 h the DCM was removed in vacuo and the residue was dissolved in EtOAc (1600 mL), washed with aqueous 1 N HCl (6×200 mL), water (200 mL), saturated aqueous NaHCO₃ (6×200 mL) and water (2×200 mL). The organic layer was dried over Na₂SO₄, filtered and concentrated in vacuo to provide a mixture of (2R,4R)-tert-butyl 4-hydroxy-2-((5-tosyl-5H-pyrrolo[2,3-b]pyrazin-2-yl)methylcarbamoyl)pyrrolidine-1-carboxylate (70% weight purity, 294 g, 100%), EtOAc and DIEA. The material was used in next step without further purification: LC/MS (Table 3, Method b) Rₜ=1.95 min.; MS m/z: 516 (M+H)⁺.

### Step C: (2R,4R)-4-Hydroxy-N-((5-tosyl-5H-pyrrolo[2,3-b]pyrazin-2-yl)methyl)pyrrolidine-2-carboxamide hydrochloride

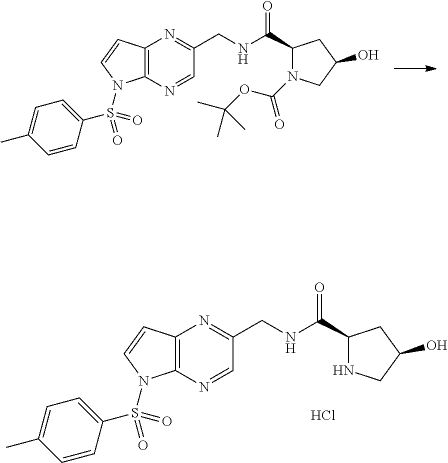 [SMILES: Cc1ccc(S(=O)(=O)n2ccc3nc(CNC(=O)[C@H]4C[C@@H](O)CN4C(=O)OC(C)(C)C)cnc32)cc1 ; Cc1ccc(S(=O)(=O)n2ccc3nc(CNC(=O)[C@H]4C[C@@H](O)CN4)cnc32)cc1 ; Cl]

To a flask containing MeOH (2100 mL) was added dropwise AcCl (348 mL, 4888 mmol) such that the internal temperature was maintained at about 5 to 10° C. After about 30 min (2R,4R)-tert-butyl 4-hydroxy-2-((5-tosyl-5H-pyrrolo[2,3-b]pyrazin-2-yl)methylcarbamoyl)pyrrolidine-1-carboxylate (420 g, 652 mmol) in MeOH (2200 mL) was added over about 30 min. The mixture was allowed to warm to rt and after about 40 h the mixture was partially concentrated in vacuo, removing ˜3500 mL of solvent. The resulting suspension was diluted with Et₂O (400 mL) and the solid was collected by filtration and the filter pad was washed with Et₂O/MeOH 1:1 (3×120 mL), Et₂O (3×120 mL), pet ether (100 mL) and dried in vacuo to provide (2R,4R)-4-hydroxy-N-((5-tosyl-5H-pyrrolo[2,3-b]pyrazin-2-yl)methyl)pyrrolidine-2-carboxamide, hydrochloride (265 g, 90%): LC/MS (Table 3, Method b) Rₜ=1.41 min.; MS m/z: 416 (M+H)⁺.

### Step D: (3R,5R)-1-Acetyl-5-(((5-tosyl-5H-pyrrolo[2,3-b]pyrazin-2-yl)methyl)carbamoyl) pyrrolidin-3-yl acetate

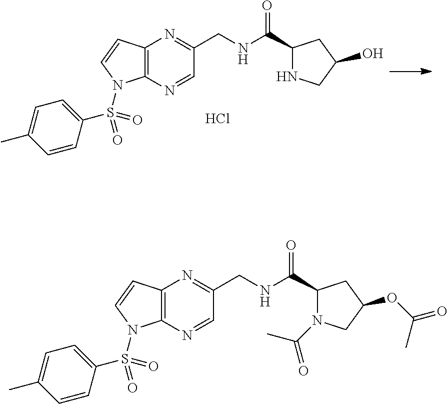 [SMILES: Cc1ccc(S(=O)(=O)n2ccc3nc(CNC(=O)[C@H]4C[C@@H](O)CN4)cnc32)cc1 ; Cl ; CC(=O)O[C@@H]1C[C@H](C(=O)NCc2cnc3c(ccn3S(=O)(=O)c3ccc(C)cc3)n2)N(C(C)=O)C1]

To a mixture of (2R,4R)-4-hydroxy-N-((5-tosyl-5H-pyrrolo[2,3-b]pyrazin-2-yl)methyl) pyrrolidine-2-carboxamide, hydrochloride (265 g, 551 mmol) in DCE (2800 mL) under nitrogen at rt was added TEA (307 mL, 2205 mmol). Ac₂O (416 mL, 4410 mmol) was added and the mixture was stirred at about 70° C. After about 24 h the solvents were removed in vacuo and to the residue was added saturated aqueous NaHCO₃ (500 mL) with stirring. After about 30 min, EtOAc (1000 mL) was added and the layers were separated. The aqueous phase was further extracted with EtOAc (3×500 mL). The extracts were combined, washed with water (2×500 mL) and brine (500 mL) and dried over Na₂SO₄, filtered and the solvent was removed in vacuo to provide (3R,5R)-1-acetyl-5-(((5-tosyl-5H-pyrrolo[2,3-b]pyrazin-2-yl)methyl)carbamoyl)pyrrolidin-3-yl acetate (270 g, 98%): LC/MS (Table 3, Method b) Rₜ=1.74 min.; MS m/z: 500 (M+H)⁺.

### Step E: (3R,5R)-1-Acetyl-5-(6-tosyl-6H-imidazo[1,5-a]pyrrolo[2,3-e]pyrazin-1-yl)pyrrolidin-3-yl acetate

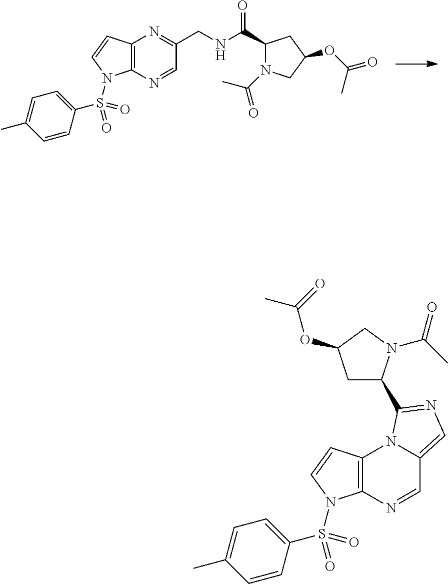 [SMILES: CC(=O)O[C@@H]1C[C@H](C(=O)NCc2cnc3c(ccn3S(=O)(=O)c3ccc(C)cc3)n2)N(C(C)=O)C1 ; CC(=O)O[C@@H]1C[C@H](c2ncc3cnc4c(ccn4S(=O)(=O)c4ccc(C)cc4)n23)N(C(C)=O)C1]

To a solution of (3R,5R)-1-acetyl-5-((5-tosyl-5H-pyrrolo[2,3-b]pyrazin-2-yl)methylcarbamoyl)pyrrolidin-3-yl acetate (275 g, 551 mmol) in TFA (679 mL, 8808 mmol), was added TFAA (692 mL, 4955 mmol). The mixture was heated to about 40° C. for about 24 h. The mixture was concentrated in vacuo and then added to a 1:1 mixture of ice and water (3000 mL) with mechanical stirring. The pH of the solution was adjusted to about 10 by the addition of solid Na₂CO₃. The aqueous mixture was extracted with DCM (4×500 mL) and the combined extracts were washed with water (200 mL), dried over Na₂SO₄, filtered, and concentrated in vacuo. To the residue was added DCM (200 mL) and the mixture was filtered and concentrated in vacuo. The crude product was purified by chromatography on silica gel (eluting with 0 to 5% MeOH in DCM) to provide (3R,5R)-1-acetyl-5-(6-tosyl-6H-imidazo[1,5-a]pyrrolo[2,3-e]pyrazin-1-yl)pyrrolidin-3-yl acetate (150 g, 57%): LC/MS (Table 3, Method b) Rₜ=1.79 min.; MS m/z: 482 (M+H)⁺.

### Step F: (3R,5R)-1-Acetyl-5-(3-bromo-6-tosyl-6H-imidazo[1,5-a]pyrrolo[2,3-e]pyrazin-1-yl)pyrrolidin-3-yl acetate

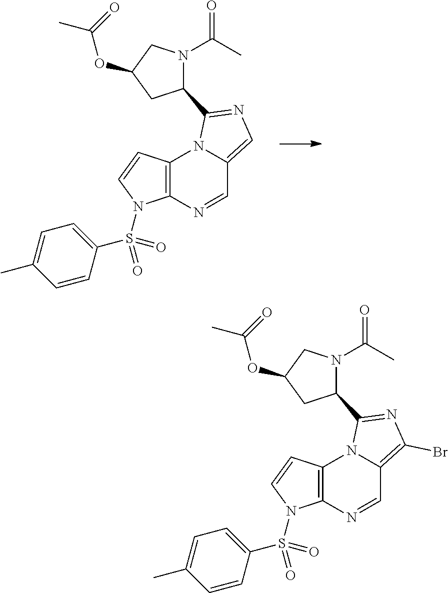 [SMILES: CC(=O)O[C@@H]1C[C@H](c2ncc3cnc4c(ccn4S(=O)(=O)c4ccc(C)cc4)n23)N(C(C)=O)C1 ; CC(=O)O[C@@H]1C[C@H](c2nc(Br)c3cnc4c(ccn4S(=O)(=O)c4ccc(C)cc4)n23)N(C(C)=O)C1]

To a solution of (3R,5R)-1-acetyl-5-(6-tosyl-6H-imidazo[1,5-a]pyrrolo[2,3-e]pyrazin-1-yl)pyrrolidin-3-yl acetate (150 g, 293 mmol) in anhydrous THF (1.9 L) at about −40° C. was added a solution of NBS (46.9 g, 264 mmol) in THF (960 mL) drop wise over about 120 min. After about 30 min an additional amount NBS (2.085 g, 11.71 mmol) in anhydrous THF (50 mL) was added drop wise. Following the addition, the mixture was allowed to warm to about −15° C. The pH of the mixture was adjusted to about 8 by the addition of saturated aqueous NaHCO₃ (500 mL). The mixture was warmed to rt filtered and partially concentrated in vacuo. To the crude mixture was added water (500 mL) and EtOAc (400 mL). The organic layer was separated and the aqueous phase was extracted with EtOAc (3×150 mL). The organic extracts were combined, washed with 5% aqueous NaHCO₃ (600 mL), water (2×300 mL), brine (400 mL), dried over Na₂SO₄, filtered and concentrated in vacuo. To the residue was added IPA (140 mL). The mixture was heated to reflux for about 15 min and then cooled to about 15° C. The solids were collected by filtration and washed with cold IPA (3×50 mL), pet ether (2×100 mL) and dried in vacuo to provide (3R,5R)-1-acetyl-5-(3-bromo-6-tosyl-6H-imidazo[1,5-a]pyrrolo[2,3-e]pyrazin-1-yl)pyrrolidin-3-yl acetate (131 g, 78%): LC/MS (Table 3, Method b) Rₜ=2.03 min.; MS m/z: 562, 563; 1:1 (M+H)⁺.

### Preparation #2. Preparation of (3R,5R)-1-acetyl-5-(3-bromo-8-methyl-6-tosyl-6H-imidazo[1,5-a]pyrrolo[2,3-e]pyrazin-1-yl)pyrrolidin-3-yl acetate

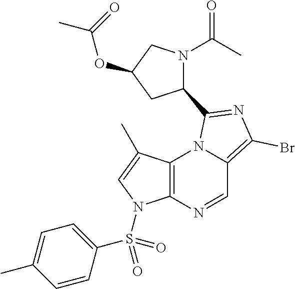 [SMILES: CC(=O)O[C@@H]1C[C@H](c2nc(Br)c3cnc4c(c(C)cn4S(=O)(=O)c4ccc(C)cc4)n23)N(C(C)=O)C1]

### Step A: tert-Butyl (5-tosyl-5H-pyrrolo[2,3-b]pyrazin-2-yl)methylcarbamate

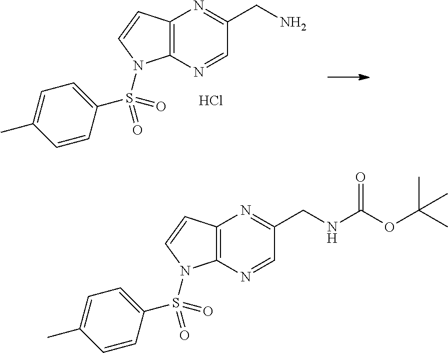 [SMILES: Cc1ccc(S(=O)(=O)n2ccc3nc(CN)cnc32)cc1 ; Cl ; Cc1ccc(S(=O)(=O)n2ccc3nc(CNC(=O)OC(C)(C)C)cnc32)cc1]

Boc₂O (10.8 mL, 46.5 mmol) and (5-tosyl-5H-pyrrolo[2,3-b]pyrazin-2-yl)methanamine, hydrochloric acid (15.0 g, 44.3 mmol, US 2011/0311474, Example #5, Step C) were combined in DMF (169 ml). TEA (13 ml, 93 mmol) was added slowly and the mixture was stirred at rt for about 1 h. The mixture was concentrated in vacuo and diluted with 300 mL of water to form a suspension. The mixture was stirred rapidly with a magnetic stirrer and manual agitation and the resulting solid was collected by filtration, washed with water (100 mL), and dried in vacuo to provide 17.8 g of tert-butyl (5-tosyl-5H-pyrrolo[2,3-b]pyrazin-2-yl)methylcarbamate that was used directly in the next step. LC/MS (Table 3, Method a) Rₜ=2.49 min; MS m/z 403 (M+H)⁺.

### Step B: tert-Butyl (5H-pyrrolo[2,3-b]pyrazin-2-yl)methylcarbamate

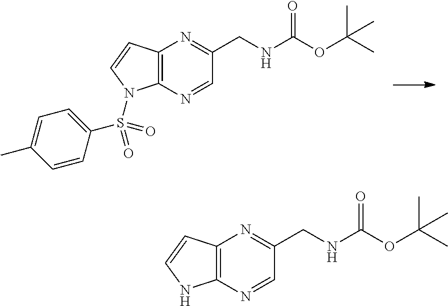 [SMILES: Cc1ccc(S(=O)(=O)n2ccc3nc(CNC(=O)OC(C)(C)C)cnc32)cc1 ; CC(C)(C)OC(=O)NCc1cnc2[nH]ccc2n1]

tert-Butyl (5-tosyl-5H-pyrrolo[2,3-b]pyrazin-2-yl)methylcarbamate (17.8 g, 44.3 mmol) was stirred in 1,4-dioxane (141 mL) and a solution of NaOH (7.08 g, 177 mmol) dissolved in water (35.1 mL) was added. The mixture heated at about 70° C. for about 1 h. The mixture was cooled to rt and was concentrated in vacuo. The residue was partitioned between EtOAc (300 mL) and water (200 mL). The pH of the water layer was adjusted to about 1 and the layers were separated. The aqueous layer was extracted with EtOAc (2×100 mL). The pH of the aqueous layer was adjusted to about 8 with saturated aqueous NaHCO₃ (˜200 mL). The aqueous layer was then extracted with DCM (100 mL). All the organic extracts were combined and washed with saturated aqueous NaHCO₃ (2×75 mL). The combined extracts were then washed with brine (50 mL), dried over Na₂SO₄, filtered, and concentrated in vacuo. Heptane (200 mL) was added to the residue and the resulting solid was collected by filtration and washed with heptane (50 mL) to provide tert-butyl (5H-pyrrolo[2,3-b]pyrazin-2-yl) (9.94 g, 90%): LCMS (Table 3, Method a) Rₜ=1.75 min.; MS m/z: 249 (M+H)⁺.

### Step C: tert-Butyl (7-bromo-5H-pyrrolo[2,3-b]pyrazin-2-yl)methylcarbamate

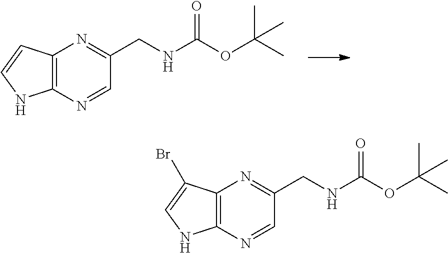 [SMILES: CC(C)(C)OC(=O)NCc1cnc2[nH]ccc2n1 ; CC(C)(C)OC(=O)NCc1cnc2[nH]cc(Br)c2n1]

To a mixture of tert-Butyl (5H-pyrrolo[2,3-b]pyrazin-2-yl)methylcarbamate (5.0 g, 20 mmol) was DMF (101 ml) was added NBS (3.58 g, 20.1 mmol) in small portions. The mixture was stirred at rt for about 1 h. The mixture was concentrated in vacuo, diluted with water (100 mL) and stirred for about 2 h. The resulting solid was collected by filtration and washed with water (50 mL) and dried in vacuo to provide tert-butyl (7-bromo-5H-pyrrolo[2,3-b]pyrazin-2-yl)methylcarbamate (6.44 g, 98%): LCMS (Table 3, Method a) Rₜ=1.99 min.; MS m/z: 327, 329 (M+H)⁺.

### Step D: tert-Butyl (7-bromo-5-tosyl-5H-pyrrolo[2,3-b]pyrazin-2-yl)methylcarbamate

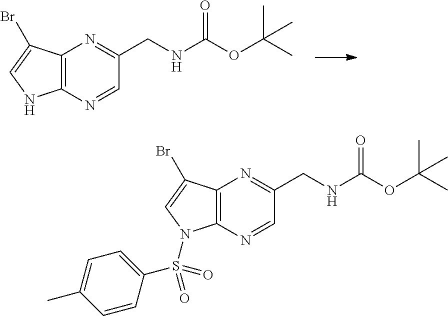

To a mixture of tert-Butyl (7-bromo-5H-pyrrolo[2,3-b]pyrazin-2-yl)methylcarbamate (6.44 g, 19.7 mmol) in DMF (131 ml) was added NaH (60% in mineral oil, (0.787 g, 19.7 mmol). The mixture was stirred at rt for about 1 h. TsCl (3.75 g, 19.7 mmol) was added and the mixture was stirred at rt for about 18 h. The mixture was concentrated in vacuo and diluted with water (250 mL). The suspension was stirred for about 4 h with magnetic stirring and manual agitation. The resulting solid was collected by filtration, washed with water (50 mL), heptane (50 mL) and dried in vacuo to provide tert-butyl (7-bromo-5-tosyl-5H-pyrrolo[2,3-b]pyrazin-2-yl)methylcarbamate (9.39 g, 99%): LC/MS (Table 3, Method a) Rₜ=2.76 min.; MS m/z: 481, 483 (M+H)⁺.

### Step E: tert-Butyl (7-methyl-5-tosyl-5H-pyrrolo[2,3-b]pyrazin-2-yl)methylcarbamate

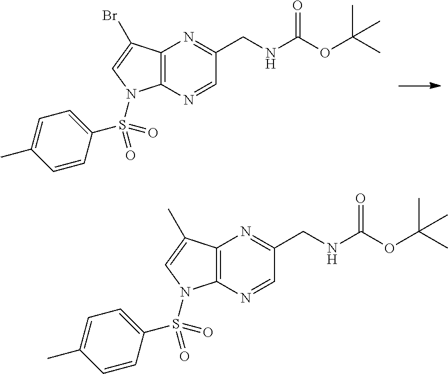

tert-Butyl (7-bromo-5-tosyl-5H-pyrrolo[2,3-b]pyrazin-2-yl)methylcarbamate (2.00 g, 4.15 mmol), Pd₂dba₃ (0.380 g, 0.415 mmol), and Cs₂CO₃ (2.71 g, 8.31 mmol) were combined in 1,4-dioxane (83 mL). The mixture was sparged by bubbling nitrogen directly into the mixture. After about 15 min, trimethylboroxine (0.752 ml, 5.40 mmol) and tricyclohexylphosphine (20% in toluene, 1.05 mL, 0.665 mmol) were added. The sparging was continued for about 15 min. The mixture was then heated at about 80° C. for about 18 h. The mixture was cooled to rt and filtered through Celite®, washing with the aid of EtOAc (100 mL). The solvents were removed in vacuo. The crude material was purified via silica gel chromatography (10 to 80% EtOAc in heptane). The material was washed with heptane (10 mL), and dried in vacuo to provide tert-butyl (7-methyl-5-tosyl-5H-pyrrolo[2,3-b]pyrazin-2-yl)methylcarbamate (1.13 g, 65%): LC/MS (Table 3, Method a) Rₜ=2.58 min.; MS m/z: 417 (M+H)⁺.

### Step F: (7-Methyl-5-tosyl-5H-pyrrolo[2,3-b]pyrazin-2-yl)methanamine

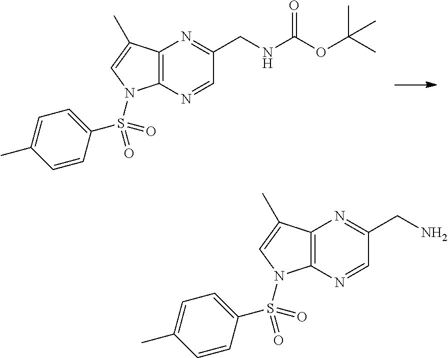

tert-Butyl (7-methyl-5-tosyl-5H-pyrrolo[2,3-b]pyrazin-2-yl)methylcarbamate (1.13 g, 2.71 mmol) was stirred in DCM (13.6 ml) and then TFA (13.6 ml, 176 mmol) was added slowly. The mixture was stirred at rt for about 5 h. The mixture was concentrated in vacuo and the residue was partitioned between DCM (50 mL) and aqueous NaHCO₃ (10 mL). The layers were separated and the aqueous layer was extracted with DCM (2×10 mL). The combined organic layers were dried over Na₂SO₄, filtered, and concentrated in vacuo to provide (7-methyl-5-tosyl-5H-1-pyrrolo[2,3-b]pyrazin-2-yl)methanamine (0.734 g, 86%): LCMS (Table 3, Method a) Rₜ=1.62 min.; MS m/z: 317 (M+H)⁺.

### Step G: (2R,4R)-tert-Butyl 4-hydroxy-2-((7-methyl-5-tosyl-5H-pyrrolo[2,3-b]pyrazin-2-yl)methylcarbamoyl)pyrrolidine-1-carboxylate

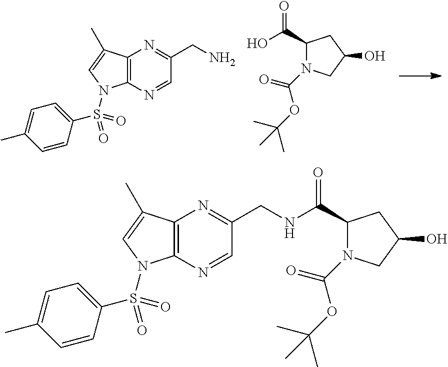

(7-Methyl-5-tosyl-5H-pyrrolo[2,3-b]pyrazin-2-yl)methanamine (0.734 g, 2.32 mmol), (2R,4R)-1-(tert-butoxycarbonyl)-4-hydroxypyrrolidine-2-carboxylic acid (0.536 g, 2.32 mmol, Preparation #1, Step A) and HATU (0.926 g, 2.44 mmol) were combined in DCM (11.6 ml). DIEA (1.01 mL, 5.80 mmol) was added slowly and the mixture was stirred at rt for about 18 h. The mixture was diluted with DCM (50 mL) and then washed with saturated aqueous NaHCO₃ (15 mL) and brine (15 mL). The organic phase was washed with 1 N HCl (15 mL), brine (15 mL), dried over Na₂SO₄, filtered, and concentrated in vacuo. The residue was purified by silica gel chromatography (1 to 5% MeOH in DCM) to provide (2R,4R)-tert-butyl 4-hydroxy-2-((7-methyl-5-tosyl-5H-pyrrolo[2,3-b]pyrazin-2-yl)methylcarbamoyl)pyrrolidine-1-carboxylate (1.39 g, 113%): LC/MS (Table 3, Method a) Rₜ=2.07 min.; MS m/z: 530 (M+H)⁺.

### Step H: (3R,5R)-1-Acetyl-5-(8-methyl-6-tosyl-6H-imidazo[1,5-a]pyrrolo[2,3-e]pyrazin-1-yl)pyrrolidin-3-yl acetate

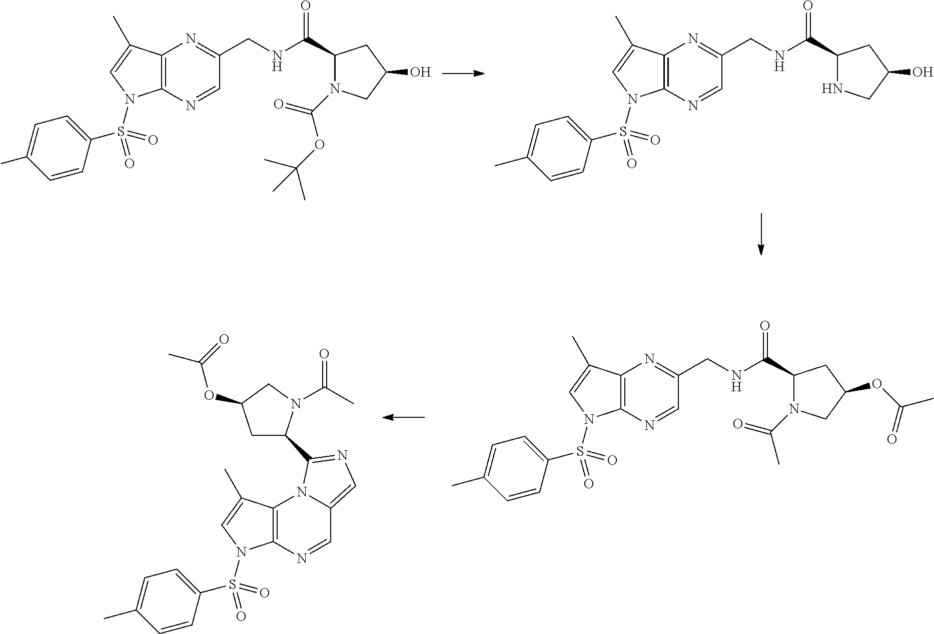 [SMILES: Cc1ccc(S(=O)(=O)n2cc(C)c3nc(CNC(=O)[C@H]4C[C@@H](O)CN4C(=O)OC(C)(C)C)cnc32)cc1 ; Cc1ccc(S(=O)(=O)n2cc(C)c3nc(CNC(=O)[C@H]4C[C@@H](O)CN4)cnc32)cc1 ; CC(=O)O[C@@H]1C[C@H](c2ncc3cnc4c(c(C)cn4S(=O)(=O)c4ccc(C)cc4)n23)N(C(C)=O)C1 ; CC(=O)O[C@@H]1C[C@H](C(=O)NCc2cnc3c(n2)c(C)cn3S(=O)(=O)c2ccc(C)cc2)N(C(C)=O)C1]

AcCl (8.73 mL, 123 mmol) was added dropwise over several minutes to MeOH (49 mL) about 0° C. The mixture was stirred at rt for about 30 min and then added to (2R,4R)-tert-butyl 4-hydroxy-2-((7-methyl-5-tosyl-5H-pyrrolo[2,3-b]pyrazin-2-yl)methylcarbamoyl)pyrrolidine-1-carboxylate (1.3 g, 2.5 mmol). The mixture was stirred at rt for about 1 h and then concentrated in vacuo. Ac₂O (20 mL) was added and the mixture was heated at about 75° C. for about 43 h. The mixture was concentrated in vacuo and TFA (15 mL) was added. TFAA (15 mL) was added and the mixture was heated to about 40° C. for about 24 h. The mixture was concentrated in vacuo and then partitioned between DCM (25 mL) and saturated aqueous NaHCO₃ (15 mL). The aqueous layer was separated and extracted with DCM (2×10 mL). The combined organic phases were dried over Na₂SO₄, filtered, and concentrated in vacuo. The residue was purified by silica gel chromatography (1 to 5% MeOH in DCM) to provide (3R,5R)-1-acetyl-5-(8-methyl-6-tosyl-6H-imidazo[1,5-c]pyrrolo[2,3-e]pyrazin-1-yl)pyrrolidin-3-yl acetate (0.746 g, 61%): LC/MS (Table 3, Method a) Rₜ=1.92 min.; MS m/z: 496 (M+H)⁺.

### Step I: (3R,5R)-1-Acetyl-5-(3-bromo-8-methyl-6-tosyl-6H-imidazo[1,5-a]pyrrolo[2,3-e]pyrazin-1-yl)pyrrolidin-3-yl acetate

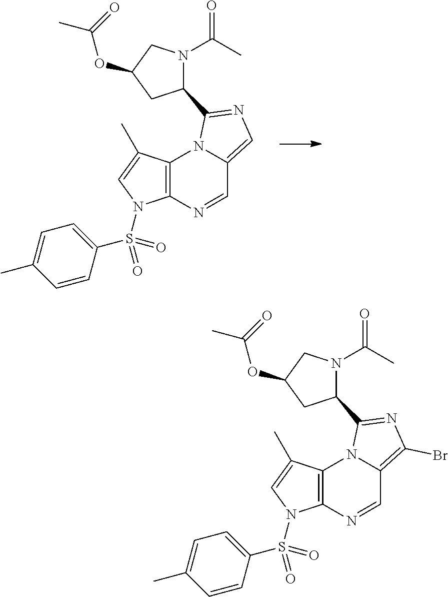 [SMILES: CC(=O)O[C@@H]1C[C@H](c2ncc3cnc4c(c(C)cn4S(=O)(=O)c4ccc(C)cc4)n23)N(C(C)=O)C1 ; CC(=O)O[C@@H]1C[C@H](c2nc(Br)c3cnc4c(c(C)cn4S(=O)(=O)c4ccc(C)cc4)n23)N(C(C)=O)C1]

To a mixture of (3R,5R)-1-acetyl-5-(8-methyl-6-tosyl-6H-imidazo[1,5-a]pyrrolo[2,3-e]pyrazin-1-yl)pyrrolidin-3-yl acetate (0.746 g, 1.51 mmol) in THF (15 mL) was added NBS (0.268 g, 1.51 mmol) in portions over about 10 min. The mixture was stirred at rt for about 20 h. The mixture was concentrated in vacuo and EtOAc (20 mL) was added. The organic layer was washed with saturated aqueous NaHCO₃ (5 mL) and water (5 mL) brine (5 mL), dried over Na₂SO₄, filtered and concentrated in vacuo. The residue was purified by silica gel chromatography (1 to 5% MeOH in DCM) to provide (3R,5R)-1-acetyl-5-(3-bromo-8-methyl-6-tosyl-6H-imidazo[1,5-a]pyrrolo[2,3-e]pyrazin-1-yl)pyrrolidin-3-yl acetate (0.753 g, 87%): LC/MS (Table 3, Method a) Rₜ=2.21 min.; MS m/z: 573, 575 (M+H)⁺.

### Preparation #3. Preparation of (3R,5R)-1-acetyl-5-(3-bromo-8-chloro-6-tosyl-6H-imidazo[1,5-a]pyrrolo[2,3-e]pyrazin-1-yl)pyrrolidin-3-yl acetate

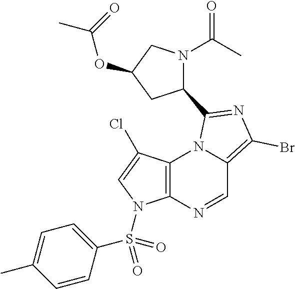 [SMILES: CC(=O)O[C@@H]1C[C@H](c2nc(Br)c3cnc4c(c(Cl)cn4S(=O)(=O)c4ccc(C)cc4)n23)N(C(C)=O)C1]

### Step A: tert-Butyl (7-chloro-5H-pyrrolo[2,3-b]pyrazin-2-yl)methylcarbamate

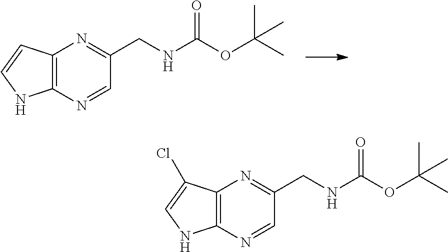 [SMILES: CC(C)(C)OC(=O)NCc1cnc2[nH]ccc2n1 ; CC(C)(C)OC(=O)NCc1cnc2[nH]cc(Cl)c2n1]

tert-Butyl (5H-pyrrolo[2,3-b]pyrazin-2-yl)methylcarbamate (4.73 g, 19.06 mmol, Preparation #2, Step B) was stirred in DMF (95 mL). NCS (2.55 g, 19.06 mmol) was added and the mixture heated at about 50° C. for about 18 h. The DMF was removed under high vacuum. The residue was suspended in water (100 mL) and scraped off the sides of the flask. The mixture was then stirred rapidly for about 3 h. The resulting solid was collected by vacuum filtration and washed with water (25 mL) to provide tert-butyl (7-chloro-5H-pyrrolo[2,3-b]pyrazin-2-yl)methylcarbamate (5.21 g, 97%): LC/MS (Table 3, Method a) Rₜ=1.95 min.; MS m/z: 283 and 285 (M+H)⁺.

### Step B: tert-butyl ((7-chloro-5-tosyl-5H-pyrrolo[2,3-b]pyrazin-2-yl)methyl)carbamate

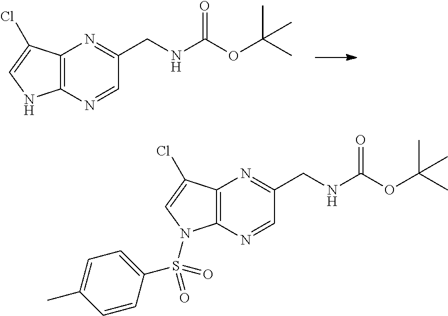

tert-Butyl (7-chloro-5H-pyrrolo[2,3-b]pyrazin-2-yl)methylcarbamate (5.21 g, 18.4 mmol) was stirred in DMF (123 mL). NaH (60% in mineral oil, 0.737 g, 18.4 mmol) was added over 10 min and the mixture was stirred at about rt for about 1 h. TsCl (3.51 g, 18.4 mmol) was added in one portion and the mixture was stirred at about rt for about 2 h. Additional NaH (0.070 g, 1.75 mmol) was added and the mixture stirred for about 20 min. Additional TsCl (350 mg, 1.84 mmol) was added and the mixture was stirred overnight. The mixture was concentrated in vacuo. The residue was taken up in water (100 mL), scraped from the sides of the flask and stirred overnight. The resulting solid was collected by filtration and washed with water (25 mL) and heptane (25 mL) to provide tert-butyl (7-chloro-5-tosyl-5H-pyrrolo[2,3-b]pyrazin-2-yl)methylcarbamate (7.81 g, 97%): LCMS (Table 3, Method a) Rₜ=2.70 min.; MS m/z: 437 and 439 (M+H)⁺.

### Step C: (7-Chloro-5-tosyl-5H-pyrrolo[2,3-b]pyrazin-2-yl)methanamine

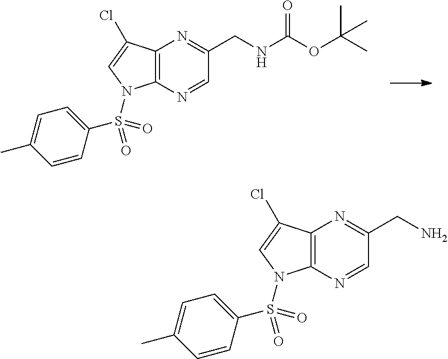

tert-Butyl (7-chloro-5-tosyl-5H-pyrrolo[2,3-b]pyrazin-2-yl)methylcarbamate (3.0 g, 6.9 mmol) was stirred in DCM (34.3 ml). TFA (34.4 ml, 446 mmol) was added carefully and the mixture stirred at rt for about 18 h. The mixture was concentrated in vacuo. The residue was taken up in a mixture of saturated aqueous NaHCO₃ (35 mL) and water (25 mL) and the mixture stirred. The resulting solid was collected by vacuum filtration and washed with water (15 mL) to provide (7-chloro-5-tosyl-5H-pyrrolo[2,3-b]pyrazin-2-yl)methanamine (2.47 g, 107%): LC/MS (Table 3, Method a) Rₜ=1.81 min.; MS m/z: 337 and 339 (M+H)⁺.

### Step D: (2R,4R)-tert-Butyl 2-((7-chloro-5-tosyl-5H-pyrrolo[2,3-b]pyrazin-2-yl)methylcarbamoyl)-4-hydroxypyrrolidine-1-carboxylate

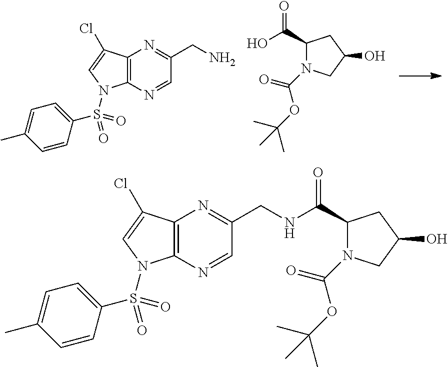

(7-Chloro-5-tosyl-5H-pyrrolo[2,3-b]pyrazin-2-yl)methanamine (2.31 g, 6.86 mmol), (2R,4R)-1-(tert-butoxycarbonyl)-4-hydroxypyrrolidine-2-carboxylic acid (1.59 g, 6.86 mmol, Preparation #1, Step A), and HATU (2.74 g, 7.20 mmol) were combined in DCM (11.60 mL. DIEA (2.99 mL, 17.2 mmol) was added slowly and the mixture stirred at rt for about 18 h. The mixture was diluted with DCM (100 mL). The mixture was washed with a mixture of saturated aqueous NaHCO₃ (30 mL) and water (30 mL). The DCM was then washed with brine (15 mL), dried over Na₂SO₄, filtered, and concentrated in vacuo. The residue was purified by flash column chromatography eluting with 1 to 5% MeOH in DCM to provide (2R,4R)-tert-butyl 2-((7-chloro-5-tosyl-5H-pyrrolo[2,3-b]pyrazin-2-yl)methylcarbamoyl)-4-hydroxypyrrolidine-1-carboxylate (5.11 g, 136%): LC/MS (Table 3, Method a) Rₜ=2.16 min.; MS m/z: 550 and 552 (M+H)⁺.

### Step E: (3R,5R)-1-Acetyl-5-(8-chloro-6-tosyl-6H-imidazo[1,5-a]pyrrolo[2,3-e]pyrazin-1-yl)pyrrolidin-3-yl acetate

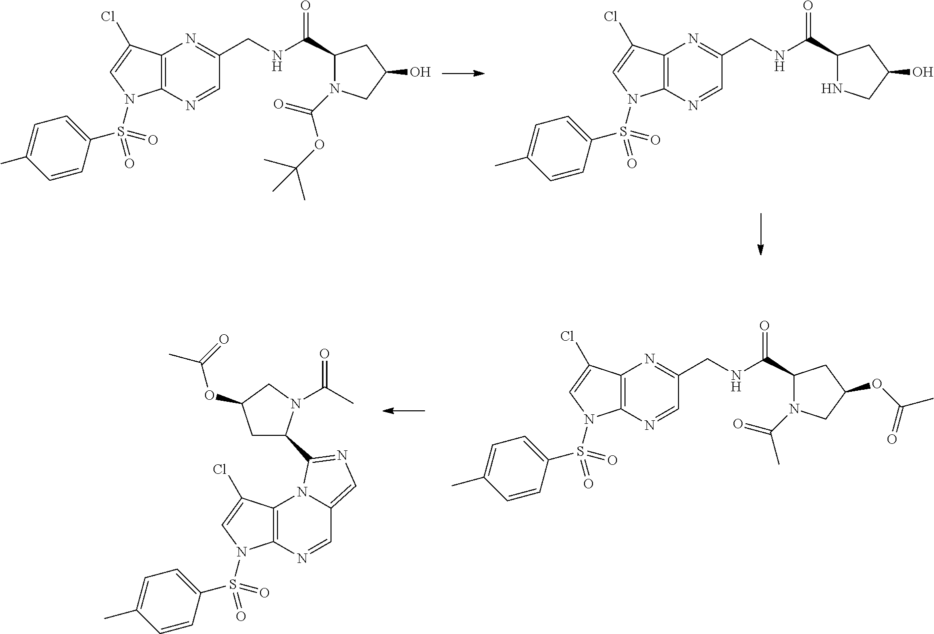 [SMILES: Cc1ccc(S(=O)(=O)n2cc(Cl)c3nc(CNC(=O)[C@H]4C[C@@H](O)CN4C(=O)OC(C)(C)C)cnc32)cc1 ; Cc1ccc(S(=O)(=O)n2cc(Cl)c3nc(CNC(=O)[C@H]4C[C@@H](O)CN4)cnc32)cc1 ; CC(=O)O[C@@H]1C[C@H](c2ncc3cnc4c(c(Cl)cn4S(=O)(=O)c4ccc(C)cc4)n23)N(C(C)=O)C1 ; CC(=O)O[C@@H]1C[C@H](C(=O)NCc2cnc3c(n2)c(Cl)cn3S(=O)(=O)c2ccc(C)cc2)N(C(C)=O)C1]

AcCl (24.4 mL, 343 mmol) was added slowly over several minutes to MeOH (137 ml) that was cooled in an ice bath. The ice bath was removed and the mixtured stirred for about 20 min. A mixture of (2R,4R)-tert-butyl 2-((7-chloro-5-tosyl-5H-pyrrolo[2,3-b]pyrazin-2-yl)methylcarbamoyl)-4-hydroxypyrrolidine-1-carboxylate (3.77 g, 6.85 mmol) in MeOH (˜5 mL) was added and the mixture stirred for about 3 h. The mixture was concentrated in vacuo. The residue was mixed with Ac₂O (50 mL) and the mixture heated to about 75° C. for about 2 h. The mixture was concentrated in vacuo. TFA (20 mL) and TFAA (20 mL) were added to the residue and the mixture was heated to about 40° C. for about 40 h. The mixture was concentrated in vacuo and the residue was taken up in DCM (50 mL) and washed with a mixture of saturated aqueous NaHCO₃ (25 mL) and water (15 mL). The layers were separated and the aqueous layer extracted with DCM (2×10 mL). The combined extracts were dried over Na₂SO₄, filtered, and concentrated in vacuo. The residue was purified by silica gel flash column chromatography eluting with 1 to 5% MeOH in DCM to provide (3R,5R)-1-acetyl-5-(8-chloro-6-tosyl-6H-imidazo[1,5-a]pyrrolo[2,3-e]pyrazin-1-yl)pyrrolidin-3-yl acetate (1.57 g, 44%): LCMS (Table 3, Method a) Rₜ=2.36 min.; MS m/z: 516, 518 (M+H)⁺.

### Step F: (3R,5R)-1-Acetyl-5-(3-bromo-8-chloro-6-tosyl-6H-imidazo[1,5-a]pyrrolo[2,3-e]pyrazin-1-yl)pyrrolidin-3-yl acetate

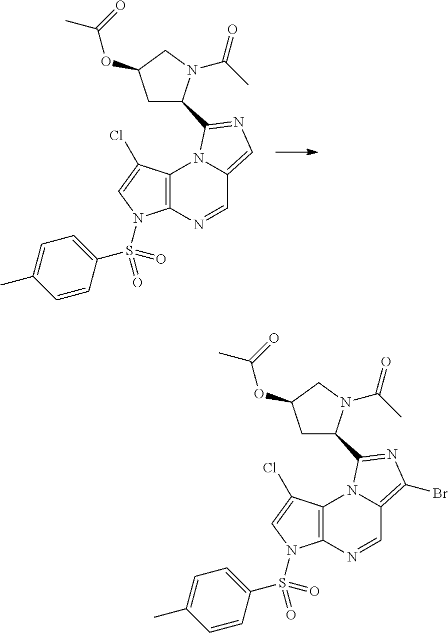 [SMILES: CC(=O)O[C@@H]1C[C@H](c2ncc3cnc4c(c(Cl)cn4S(=O)(=O)c4ccc(C)cc4)n23)N(C(C)=O)C1 ; CC(=O)O[C@@H]1C[C@H](c2nc(Br)c3cnc4c(c(Cl)cn4S(=O)(=O)c4ccc(C)cc4)n23)N(C(C)=O)C1]

(3R,5R)-1-Acetyl-5-(8-chloro-6-tosyl-6H-imidazo[1,5-a]pyrrolo[2,3-e]pyrazin-1-yl)pyrrolidin-3-yl acetate (1.57 g, 3.04 mmol) was stirred in THF (30.4 mL) and NBS (0.542 g, 3.04 mmol) was added in small portions over a few minutes. The mixture was stirred at rt for about 2 h. The solvents were removed in vacuo. The residue was partitioned between EtOAc (50 mL) and saturated aqueous NaHCO₃ (10 mL) and water (10 mL) and the layers separated. The combined extracts were dried over Na₂SO₄, filtered, and concentrated in vacuo. The residue was purified by silica gel flash column chromatography eluting with 1 to 5% MeOH in DCM to provide (3R,5R)-1-acetyl-5-(3-bromo-8-chloro-6-tosyl-6H-imidazo[1,5-a]pyrrolo[2,3-e]pyrazin-1-yl)pyrrolidin-3-yl acetate (1.78 g, 98%): LC/MS (Table 3, Method a) Rₜ=2.26 min.; MS m/z: 595, 597 (M+H)⁺.

### Preparation #4: 2-(2-(4-(4,4,5,5-Tetramethyl-1,3,2-dioxaborolan-2-yl)phenoxy)ethyl)-2H-1,2,3-triazole

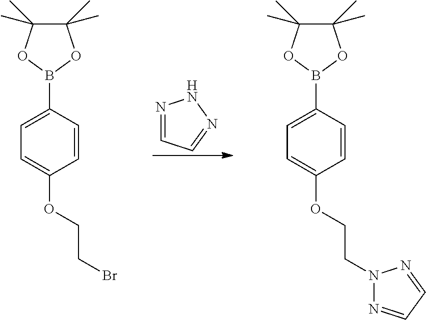 [SMILES: CC1(C)OB(c2ccc(OCCBr)cc2)OC1(C)C ; c1cn[nH]n1 ; CC1(C)OB(c2ccc(OCCn3nccn3)cc2)OC1(C)C]

To a flask was added 2-(4-(2-bromoethoxy)phenyl)-4,4,5,5-tetramethyl-1,3,2-dioxaborolane (0.750 g, 2.293 mmol, Combi Blocks), KI (0.057 g, 0.344 mmol) and K₂CO₃ (1.27 g, 9.17 mmol) in DMSO (10 mL) followed by 2H-1,2,3-triazole (0.317 g, 4.59 mmol, TCI). The mixture was heated to about 80° C. for about 1 h then cooled to rt. The mixture was filtered and the filtrate was concentrated in vacuo. The crude material was purified by silica gel flash chromatography eluting with 0 to 50% EtOAc/DCM to provide 2-(2-(4-(4,4,5,5-tetramethyl-1,3,2-dioxaborolan-2-yl)phenoxy)ethyl)-2H-1,2,3-triazole (0.313 g, 43%): LC/MS (Table 3, Method e) Rₜ=0.74 min; MS m/z: 316 (M+H)⁺.

### Preparation #5: (3R,5R)-5-(3-(4-Methoxyphenyl)-6H-imidazo[1,5-a]pyrrolo[2,3-e]pyrazin-1-yl)pyrrolidin-3-ol

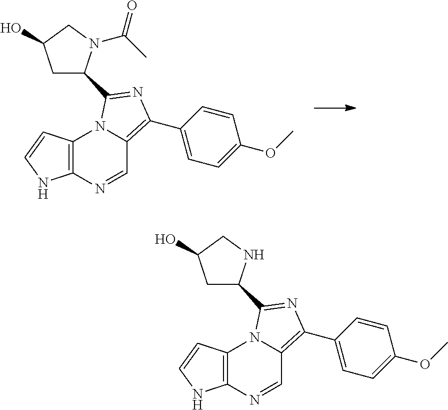 [SMILES: COc1ccc(-c2nc([C@H]3C[C@@H](O)CN3C(C)=O)n3c2cnc2[nH]ccc23)cc1 ; COc1ccc(-c2nc([C@H]3C[C@@H](O)CN3)n3c2cnc2[nH]ccc23)cc1]

A mixture of 1-((2R,4R)-4-hydroxy-2-(3-(4-methoxyphenyl)-6H-imidazo[1,5-a]pyrrolo[2,3-e]pyrazin-1-yl)pyrrolidin-1-yl)ethanone (0.2 g, 0.511 mmol, Example #F.1.4) and aqueous HCl (6 M, 2.55 mL, 15.3 mmol) in 1,4-dioxane (6 mL) was heated at about 100° C. for about 4 h. The mixture was cooled to rt and the precipitate was collected by filtration, washed with Et₂O and dried under vacuum to give (3R,5R)-5-(3-(4-methoxyphenyl)-6H-imidazo[1,5-a]pyrrolo[2,3-e]pyrazin-1-yl)pyrrolidin-3-ol (0.163 g, 92%): LC/MS (Table 3, Method a) Rₜ=1.45 min; MS m/z 350 (M+H)⁺.

### Preparation #6: (3R,5R)-5-(3-(4-(2-methoxyethoxy)phenyl)-6H-imidazo[1,5-a]pyrrolo[2,3-e]pyrazin-1-yl)pyrrolidin-3-ol

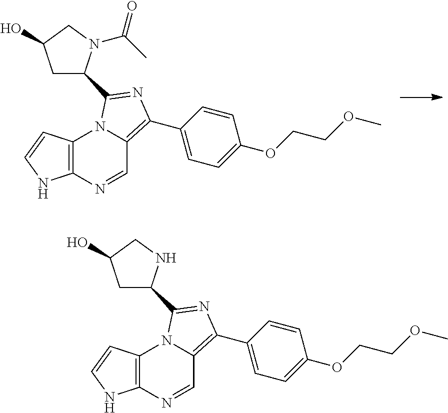 [SMILES: COCCOc1ccc(-c2nc([C@H]3C[C@@H](O)CN3C(C)=O)n3c2cnc2[nH]ccc23)cc1 ; COCCOc1ccc(-c2nc([C@H]3C[C@@H](O)CN3)n3c2cnc2[nH]ccc23)cc1]

A mixture of 1-((2R,4R)-4-hydroxy-2-(3-(4-(2-methoxyethoxy)phenyl)-6H-imidazo[1,5-c]pyrrolo[2,3-e]pyrazin-1-yl)pyrrolidin-1-yl)ethanone (0.205 g, 0.473 mmol, Prepared using A from Preparation #1 with 4-(2-methoxyethoxy)phenylboronic acid and I), aqueous HCl (6 M, 2.34 mL, 14.18 mmol) in 1,4-dioxane (5.5 mL) was heated at about 100° C. for about 3 h. The solvent was removed in vacuo. The residue was suspended in MeOH and the solid was collected by filtration, washed with Et₂O and dried under vacuum to give (3R,5R)-5-(3-(4-(2-methoxyethoxy)phenyl)-6H-imidazo[1,5-a]pyrrolo[2,3-e]pyrazin-1-yl)pyrrolidin-3-ol (0.179 g, 96%): LC/MS (Table 3, Method b) Rₜ=1.25 min; MS m/z 394 (M+H)⁺.

General Procedure A: Suzuki Reaction of an Aryl or Heteroaryl Halide with an Aryl or Heteroaryl Boronic Acid or Boronate

To a mixture of an aryl halide (preferably 1 equiv), a boronic acid or boronate ester (1 to 2 equiv, preferably 1.1 to 1.2 equiv), and an inorganic base (such as, KF, Na₂CO₃, K₂CO₃ or Cs₂CO₃, preferably Cs₂CO₃) (1.1 to 16 equiv, preferably 1.5 to 3 equiv) in a solvent (such as THF, DME, DMF, 1,4-dioxane, DME/water, 1,4-dioxane/water, toluene/EtOH/water, 1,4-dioxane/EtOH/water or water; preferably toluene/EtOH/water, DME or 1,4-dioxane) is added a palladium catalyst (for example Pd₂dba₃, Pd(PPh₃)₄, bis(acetato)triphenylphosphinepalladium(II), polymer-bound FibreCat™ 1032, PdCl₂(dppf), Pd(OAc)₂ or Pd(PPh₃)₂Cl₂; preferably PdCl₂(dppf), Pd(PPh₃)₂Cl₂, Pd(OAc)₂, Pd₂dba₃, 0.01 to 0.20 equiv, preferably 0.02 to 0.1 equiv) and a ligand (for example tricyclohexylphosphine, tri-tert-butyl-phosphine, CgPPh; preferably CgPPh, tricyclohexylphosphine; 0.01-1.0 equiv, preferably 0.16 equiv) is added optionally. The mixture is optionally evacuated and purged with nitrogen after the addition of any reagent. The mixture is then heated at about 40 to 150° C. (preferably about 65 to 120° C.) for about 1 to 48 h (preferably about 2 to 24 h) thermally, or at about 100 to 200° C. (preferably about 100 to 150° C.) for about 5 min to 6 h min (preferably about 15 to 2 h) in a microwave (preferably 2 to 10 min ramp time, 150 to 300 Watts max power, 250 psi max pressure). The mixture is allowed to cool to rt and is worked up using one of the following methods. Method 1. For reactions containing water, the mixture may optionally be filtered and is then diluted with an organic solvent (such as DCM or EtOAc). The layers are separated, the organic solution is optionally washed with water and/or brine, dried over anhydrous MgSO₄ or Na₂SO₄, filtered, and the solvent is removed in vacuo to give the desired compound. Method 2. The mixture is concentrated in vacuo and optionally purified using one or more of the Purification Methods described above to give the desired compound. Method 3. The catalyst is removed by filtration and the filtrate is concentrated in vacuo. For all of the workup methods, at any point during the work up after a residue is obtained, the residue is optionally taken up in an organic solvent (such as DCM, EtOAc, THF, preferably EtOAc) and treated with either a mercaptopropyl functionalized silica gel (0.5 to 10 equiv, preferably 0.8 to 1.5 equiv) at rt for 1 to 48 h (preferably 12 to 24 h). The mixture can then be filtered and filtrate concentrated in vacuo to give the targeted compound.

### Illustration of General Procedure A:

### Preparation #A.1: (3R,5R)-1-Acetyl-5-(3-(4-hydroxyphenyl)-6-tosyl-6H-imidazo[1,5-a]pyrrolo[2,3-e]pyrazin-1-yl)pyrrolidin-3-yl acetate

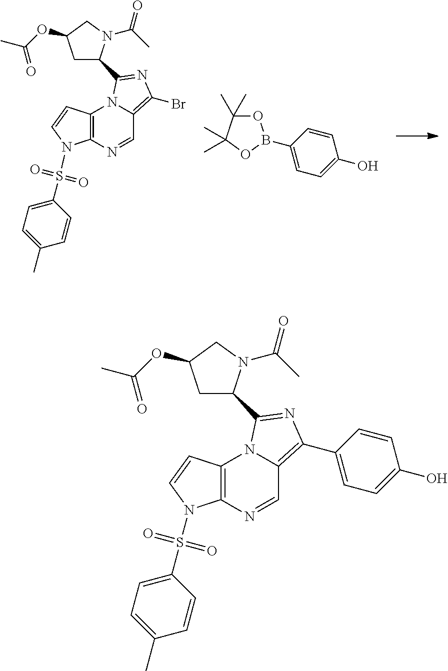 [SMILES: CC(=O)O[C@@H]1C[C@H](c2nc(Br)c3cnc4c(ccn4S(=O)(=O)c4ccc(C)cc4)n23)N(C(C)=O)C1 ; CC1(C)OB(c2ccc(O)cc2)OC1(C)C ; CC(=O)O[C@@H]1C[C@H](c2nc(-c3ccc(O)cc3)c3cnc4c(ccn4S(=O)(=O)c4ccc(C)cc4)n23)N(C(C)=O)C1]

A microwave vial was charged with (3R,5R)-1-acetyl-5-(3-bromo-6-tosyl-6H-imidazo[1,5-a]pyrrolo[2,3-e]pyrazin-1-yl)pyrrolidin-3-yl acetate (1.25 g, 2.23 mmol, Preparation #1), 4-(4,4,5,5-tetramethyl-[1,3,2]dioxaborolan-2-yl)-phenol (0.736 g, 3.35 mmol), PdCl₂(dppf) (0.182 g, 0.223 mmol), and aqueous Na₂CO₃ (2 N, 2.79 mL, 5.58 mmol) in DME (4.46 mL). The mixture was purged with argon for 1 min and then irradiated in the microwave at about 120° C. (250 psi maximum pressure, 10 min ramp, 300 max watts) for about 15 min. The mixture was concentrated in vacuo and the residue was partitioned between DCM (30 mL) and saturated aqueous NaHCO₃ (30 mL). The aqueous layer was extracted with DCM (3×30 mL). The organic layers were combined, dried over anhydrous MgSO₄, filtered, and concentrated in vacuo. The material was purified by silica gel flash chromatography eluting with 5 to 50% acetone in DCM to afford (3R,5R)-1-acetyl-5-(3-(4-hydroxyphenyl)-6-tosyl-6H-imidazo[1,5-a]pyrrolo[2,3-e]pyrazin-1-yl)pyrrolidin-3-yl acetate (1.04 g, 81%). LC/MS (Table 3, Method d) Rₜ=2.05 min.; MS m/z: 574 (M+H)⁺.

TABLE A.1
Examples prepared from 1-((2R,4R)-2-(3-Bromo-6H-imidazo[1,5-
a]pyrrolo[2,3-e]pyrazin-1-yl)-4-hydroxypyrrolidin-1-yl)ethanone (Preparation #D.1)
with an aryl or heteroaryl boronic acid or boronate using General Procedure A

| | | | Rₜ min | m/z | |
|---|---|---|---|---|---|
| Boronic acid or | | | (Table 3, | ESI+ | Syk |
| boronate | Product | Example # | Method) | (M + H)⁺ | IC₅₀ |
| Thiophen-3-ylboronic acid | 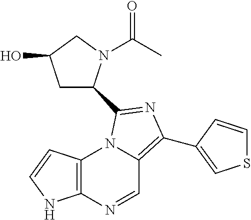 [SMILES: CC(=O)N1C[C@H](O)C[C@@H]1c1nc(-c2ccsc2)c2cnc3[nH]ccc3n12] | A.1.1 | 1.49 (b) | 368.1 | B |
| 4- Ethoxyphenylboronic acid | 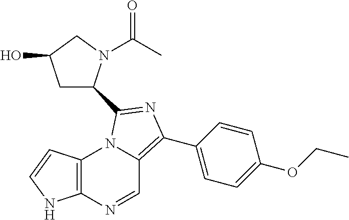 [SMILES: CCOc1ccc(-c2nc([C@H]3C[C@@H](O)CN3C(C)=O)n3c2cnc2[nH]ccc23)cc1] | A.1.2 | 1.65 (a) | 405 | B |
| 2-(2-(4-(4,4,5,5- Tetramethyl-1,3,2- dioxaborolan-2- yl)phenoxy)ethyl)-2H- 1,2,3-triazole (Preparation #4) | 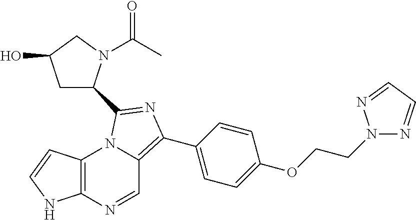 [SMILES: CC(=O)N1C[C@H](O)C[C@@H]1c1nc(-c2ccc(OCCn3nccn3)cc2)c2cnc3[nH]ccc3n12] | A.1.3 | 1.50 (a) | 473 | C |
| 4- (Trifluoromethyl)phenyl- boronic acid | 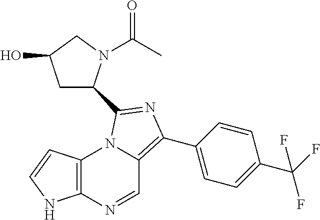 [SMILES: CC(=O)N1C[C@H](O)C[C@@H]1c1nc(-c2ccc(C(F)(F)F)cc2)c2cnc3[nH]ccc3n12] | A.1.4 | 1.80 (a) | 430 | C |
| 4- (Difluoromethyl)phenyl- boronic acid | 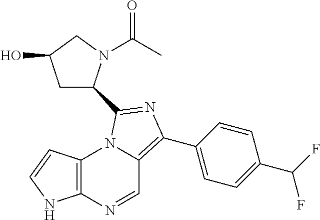 [SMILES: CC(=O)N1C[C@H](O)C[C@@H]1c1nc(-c2ccc(C(F)F)cc2)c2cnc3[nH]ccc3n12] | A.1.5 | 1.61 (a) | 412 | C |
| 4,4,5,5-Tetramethyl- 2-(4-(3-methyloxetan- 3-yl)phenyl)-1,3,2- dioxaborolane (Prepared using B from 3-(4-bromophenyl)-3- methyloxetane [WO 2009/006315, Preparation #46] with 4,4,4′,4′,5,5,5',5'- octamethyl-2,2′- bi(1,3,2- dioxaborolane) | 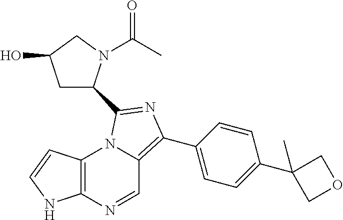 [SMILES: CC(=O)N1C[C@H](O)C[C@@H]1c1nc(-c2ccc(C3(C)COC3)cc2)c2cnc3[nH]ccc3n12] | A.1.6 | 1.67 (a) | 432 | A |

### General Procedure B: Conversion of an Aryl/Heteroaryl Halide to an Aryl/Heteroaryl Boronate

A flask is charged with an aryl or heteroaryl halide (1 equiv), and a borane or diborane (such as 4,4,5,5-tetramethyl-1,3,2-dioxaborolane or bis(pinacolato)diboron; 1 to 4 equiv, preferably 1 to 2 equiv), a base (such as TEA, KOAc, Na₂CO₃ or Cs₂CO₃, preferably KOAc, 1 to 6 equiv, preferably 1 to 3 equiv), a palladium catalyst (such as PdCl₂(dppf), Pd(OAc)₂, PdCl₂(MeCN)₂, 0.02 to 1 equiv, preferably 0.03 to 0.08 equiv), and optionally a ligand (such as SPhos, dppf, PPh₃, tricyclohexylphosphine or t-Bu₃P, preferably dppf or SPhos, 0.01 to 0.1 equiv, preferably 0.02 to 0.05 equiv) and an organic solvent (such as THF, 1,4-dioxane, DMSO or DCM, preferably 1,4-dioxane or DMSO). The mixture is heated at about 60 to 120° C. (preferably about 80 to 105° C.) for about 1 to 48 h (preferably about 2 to 24 h). The mixture is optionally concentrated in vacuo to give the targeted compound. The reaction mixture is optionally filtered through a media (such as silica gel or Celite®) which is rinsed with an appropriate solvent (such as EtOAc, 1,4-dioxane, THF, MeCN, DCM, Et₂O, MeOH, or EtOH) and then optionally concentrated in vacuo to give a residue. Either the residue or the solution may be optionally partitioned between water and an organic solvent (such as EtOAc, Et₂O or DCM). The organic layer is isolated and may be optionally washed in no particular order with water and/or aqueous solutions containing an acid (such as HCl, AcOH or NH₄Cl) and/or aqueous solutions containing a base (such as NaHCO₃, Na₂CO₃, NaOH, KOH or NH₄OH) and/or aqueous solutions containing an inorganic salt (such as NaCl, Na₂SO₃ or Na₂S₂O₃). The organic solution may then be optionally dried with a drying agent (such as anhydrous MgSO₄ or Na₂SO₄), filtered and concentrated in vacuo to give the targeted compound.

### Illustration of General Procedure B

### Preparation #B.1: 2-(4-(4,4,5,5-Tetramethyl-1,3,2-dioxaborolan-2-yl)phenyl)propan-2-ol

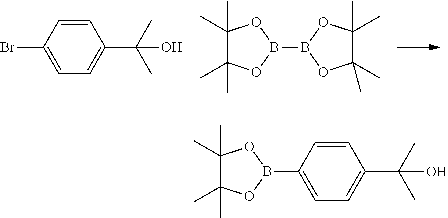

A flask was charged with 2-(4-bromophenyl)propan-2-ol (5.00 g, 23.3 mmol, Bioorg. Med. Chem. Lett. 2007, 17, 662), 4,4,4′,4′,5,5,5′,5′-octamethyl-2,2′-bi(1,3,2-dioxaborolane) (6.49 g, 25.6 mmol), KOAc (6.84 g, 69.7 mmol), PdCl₂(dppf) (0.949 g, 1.16 mmol) and DMSO (155 mL). The mixture was heated to about 80° C. for about 4 h. After cooling to rt, the mixture was partitioned between brine (400 mL) and EtOAc (100 mL). The organic layer was isolated and the aqueous phase was extracted with two further portions of EtOAc (2×50 mL). The organic layers were combined, washed with brine (5×100 mL), dried over anhydrous MgSO₄ and concentrated in vacuo. The crude material was purified by silica gel flash chromatography with a gradient of 0 to 100% EtOAc/hexanes to give 2-(4-(4,4,5,5-Tetramethyl-1,3,2-dioxaborolan-2-yl)phenyl)propan-2-ol (2.76 g, 45%): ¹H NMR (d-DMSO) δ 7.80 (s, J=8, 2H), 7.50 (s, J=8, 2H), 1.58 (s, 6H), 1.34 (s, 12H).

General Procedure C: Buchwald Reaction of an Aryl Halide with an Aniline

A mixture of an aryl halide (1.0 equiv), an aniline (1 to 2.2 equiv, preferably 1 to 1.2 equiv), a palladium catalyst (such as Pd₂dba₃, Pd(OAc)₂, preferably Pd₂dba₃ 0.01 to 1.0 equiv, preferably 0.04 to 0.1 equiv), a ligand (such as BINAP, X-Phos, Xanthphos or tert-butyl-X-Phos, preferably BINAP, tert-butyl-X-Phos or X-Phos, 0.01 to 2.0 equiv, preferably 0.04 to 0.1 equiv) and a base (such as K₂CO₃, Na₂CO₃, Cs₂CO₃, K₃PO₄, NaOt-Bu, KOt-Bu, KOAc, KOH, preferably K₂CO₃; 1 to 5 equiv, preferably 1 to 3 equiv) are added to a solvent (such as toluene, 1,4-dioxane, t-BuOH, preferably toluene). The mixture is degassed under an inert atmosphere (such as nitrogen or argon, preferably nitrogen) and heated with conventional heating at about 70 to 120° C. (preferably about 80 to 100° C.) for about 1 to 48 h (preferably about 2 to 24 h). The mixture is cooled to rt. The mixture is optionally filtered through a media (such as silica gel or Celite®) which is rinsed with an appropriate solvent (such as EtOAc, 1,4-dioxane, THF, MeCN, DCM, Et₂O, MeOH, EtOH, DMSO, 1:1 MeOH/DMSO or 2:1 MeOH/DMSO, preferably 1:1 MeOH/DMSO) and then the filtrate is optionally concentrated in vacuo or under a warm nitrogen stream to give a residue.

### Illustration of General Procedure C

### Preparation #C.1: 1-(4-Bromophenyl)-3,3-difluoroazetidine

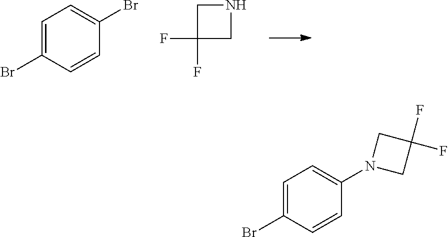 [SMILES: Brc1ccc(Br)cc1 ; FC1(F)CNC1 ; FC1(F)CN(c2ccc(Br)cc2)C1]

A flask was charged with 1,4-dibromobenzene (2.2 g, 9.33 mmol), 3,3-difluoroazetidine, hydrochloric acid (1.33 g, 10.3 mmol), BINAP (0.406 g, 0.653 mmol), Pd₂(dba)₃ (0.427 g, 0.466 mmol) and toluene (20 mL). The mixture was sparged with nitrogen for about 10 min followed by the addition of NaOt-Bu (1.97 g, 20.5 mmol). The flask was filled with nitrogen and evacuated three times and the mixture was heated to about 85° C. overnight. The mixture was cooled to rt and DCM (50 mL) and water (20 mL) were added. The layers were separated and the organic layer was dried over anhydrous MgSO₄, filtered and concentrated in vacuo. The residue was adsorbed onto silica gel (7.5 g) and purified by silica gel chromatography eluting with 0 to 50% EtOAc in heptane to give 1-(4-bromophenyl)-3,3-difluoroazetidine (0.96 g, 38%). ¹H NMR (d-DMSO) δ 7.38 (d, 2H), 6.53 (d, 2H), 4.25 (m, 4H).

General Procedure D: Removal of a Tosyl Group and an Acetate Group from a Compound Containing an N-Tosyl Group and an O-Acetate Group in One Reaction

To a solution of a compound containing an N-tosyl Group and an O-acetate group (1 equiv) in an organic solvent (such as THF, DME, EtOH, 1,4-dioxane or MeOH, preferably 1,4-dioxane or MeOH) is added an aqueous solution of a base (such as 1 to 6 N; NaOH or KOH; 2 to 20 equiv, preferably 2 to 15 equiv). To the mixture is optionally added a sulfur nucleuophile (such as cystein, tert-butylsulfide, preferably cysteine; 1 to 10 equiv, preferably 2-5 equiv). The mixture is then either heated at about 40 to 110° C. (preferably about 50 to 95° C.) in an oil bath for about 1 to 48 h (preferably about 2 to 16 h) or heated in a microwave at about 80 to 200° C. (preferably 100 to 150° C.) for about 10 to 60 min (preferably 15 to 30 min) (250 psi maximum pressure, 2 to 10 min ramp time, 150 to 300 max watts). The mixture is optionally concentrated in vacuo to give the targeted compound. The mixture is optionally filtered through a media (such as silica gel or Celite®) which is rinsed with an appropriate solvent (such as EtOAc, 1,4-dioxane, THF, MeCN, DCM, Et₂O, MeOH, or EtOH) and then optionally concentrated in vacuo to give a residue. Either the residue or the solution may be optionally partitioned between water and an organic solvent (such as EtOAc, Et₂O or DCM). The organic layer is isolated and may be optionally washed in no particular order with water and/or aqueous solutions containing an acid (such as HCl, AcOH or NH₄Cl) and/or aqueous solutions containing a base (such as NaHCO₃, Na₂CO₃, NaOH, KOH or NH₄OH) and/or aqueous solutions containing an inorganic salt (such as NaCl, Na₂SO₃ or Na₂S₂O₃). The organic solution may then be optionally dried with a drying agent (such as anhydrous MgSO₄ or Na₂SO₄), filtered and concentrated in vacuo to give the targeted compound.

### Illustration of General Procedure D:

### Preparation #D.1: 1-((2R,4R)-2-(3-Bromo-6H-imidazo[1,5-a]pyrrolo[2,3-e]pyrazin-1-yl)-4-hydroxypyrrolidin-1-yl)ethanone

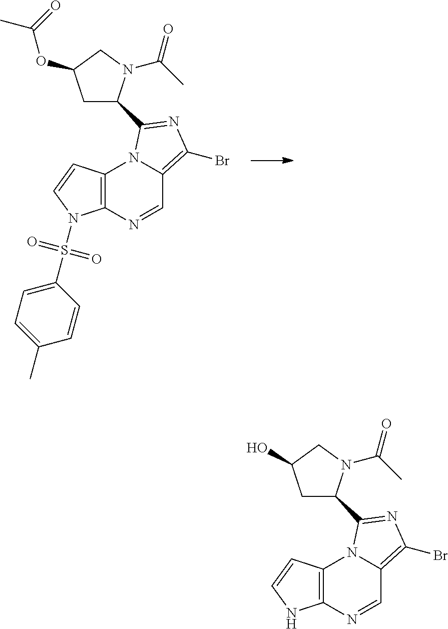 [SMILES: CC(=O)O[C@@H]1C[C@H](c2nc(Br)c3cnc4c(ccn4S(=O)(=O)c4ccc(C)cc4)n23)N(C(C)=O)C1 ; CC(=O)N1C[C@H](O)C[C@@H]1c1nc(Br)c2cnc3[nH]ccc3n12]

A flask was charged with (3R,5R)-1-acetyl-5-(3-bromo-6-tosyl-6H-imidazo[1,5-a]pyrrolo[2,3-e]pyrazin-1-yl)pyrrolidin-3-yl acetate (38.5 g, 68.6 mmol, Preparation #1) in aqueous NaOH (1 M, 412 ml, 412 mmol). The mixture was heated to about 55° C. for about 4 h. The mixture was cooled to rt and the resulting precipitate was collected by filtration and washed with water (50 mL) and dried in vacuo to afford 1-((2R,4R)-2-(3-bromo-6H-imidazo[1,5-a]pyrrolo[2,3-e]pyrazin-1-yl)-4-hydroxypyrrolidin-1-yl)ethanone (23.5 g, 94%); LC/MS (Table 3, method a) Rₜ=1.17 min.; MS m/z: 364 and 366 (M+H)⁺.

TABLE D
Examples prepared from a compound containing an N-tosyl group and an
O-actate group using General Procedure D

| Compound containing | | | Rₜ min | m/z | |
|---|---|---|---|---|---|
| an N-tosyl Group and an | | | (Table 3, | ESI+ | Syk |
| O-actate | Product | Example # | Method) | (M + H)⁺ | IC₅₀ |
| (3R,5R)-1-acetyl-5-(3-(4- ((3-methyloxetan-3- yl)methoxy)phenyl)-6- tosyl-6H-imidazo[1,5- a]pyrrolo[2,3-e]pyrazin-1- yl)pyrrolidin-3-yl acetate (Preparation #H.1) | 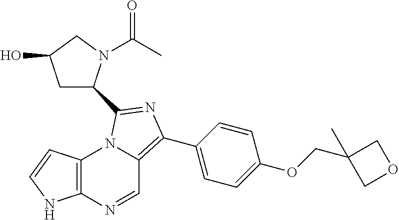 [SMILES: CC(=O)N1C[C@H](O)C[C@@H]1c1nc(-c2ccc(OCC3(C)COC3)cc2)c2cnc3[nH]ccc3n12] | D.1.1 | 1.71 (a) | 462 | B |
| (3R,5R)-1-Acetyl-5-(3-(5- (hydroxymethyl)thiophen- 3-yl)-6-tosyl-6H- imidazo[1,5-a]pyrrolo[2,3- e]pyrazin-l-yl)pyrrolidin- 3-yl acetate (prepared using A from Preparation #1 with 5- (hydroxymethyl)thiophen- 3-ylboronic acid) | 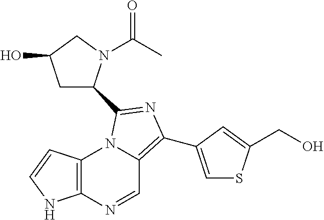 [SMILES: CC(=O)N1C[C@H](O)C[C@@H]1c1nc(-c2csc(CO)c2)c2cnc3[nH]ccc3n12] | D.1.2 | 1.22 (b) | 398.3 | B |
| (3R,5R)-1-Acetyl-5-(3-(4- (cyclopropylmethoxy)phenyl)- 6-tosyl-6H- imidazo[1,5-a]pyrrolo[2,3- e]pyrazin-1-yl)pyrrolidin- 3-yl acetate (prepared using A from Preparation #1 with 4- (cyclopropylmethoxy)phenyl- boronic [Combi- blocks]) | 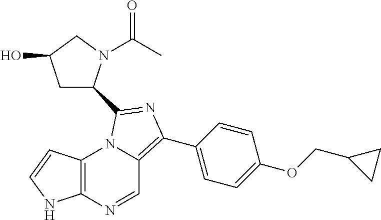 [SMILES: CC(=O)N1C[C@H](O)C[C@@H]1c1nc(-c2ccc(OCC3CC3)cc2)c2cnc3[nH]ccc3n12] | D.1.3 | 1.80 (a) | 431 | B |

General Procedure E: Formation of an Amide from an Amine and a Carboxylic Acid

To a flask is added in no particular order, a carboxylic acid or carboxylate salt (1 to 5 equiv, preferably 1.1 to 1.5 equiv) an amine (1 to 5 equiv, preferably 1 to 1.5 equiv), an organic solvent (such as DCM, DCE, THF, or 1,4-dioxane, preferably DCM), a peptide coupling reagent (such as BOP-Cl, IBCF, HATU or EDC.HCl, preferably HATU; 1 to 10 equiv, preferably 1 to 2 equiv), a base (such as TEA, DIEA, pyridine or DIEA, preferably DIEA; 1 to 20 equiv, preferably 1 to 5 equiv) and optionally HOBt (0 to 5 equiv, preferably 0 to 1 equiv). The mixture is then stirred at about 10 to 60° C. (preferably about 15 to 50° C.) for about 15 min to 48 h (preferably about 15 min to 12 h). The mixture is optionally concentrated in vacuo to give the targeted compound. The mixture is optionally filtered through a media (such as silica gel or Celite®) which is rinsed with an appropriate solvent (such as EtOAc, 1,4-dioxane, THF, MeCN, DCM, Et₂O, MeOH, EtOH) and then optionally concentrated in vacuo to give a residue. Either the residue or the solution may be optionally partitioned between water and an organic solvent (such as EtOAc, Et₂O or DCM). The organic layer is isolated and may be optionally washed in no particular order with water and/or aqueous solutions containing an acid (such as HCl, AcOH or NH₄Cl) and/or aqueous solutions containing a base (such as NaHCO₃, Na₂CO₃, NaOH, KOH or NH₄OH) and/or aqueous solutions containing an inorganic salt (such as NaCl, Na₂SO₃ or Na₂S₂O₃). The organic solution may then be optionally dried with a drying agent (such as anhydrous MgSO₄ or Na₂SO₄), filtered and concentrated in vacuo to give the targeted compound.

### Illustration of General Procedure E

### Preparation #E.1: (4,4-Difluoropiperidin-1-yl)(4-(4,4,5,5-tetramethyl-1,3,2-dioxaborolan-2-yl)phenyl)methanone

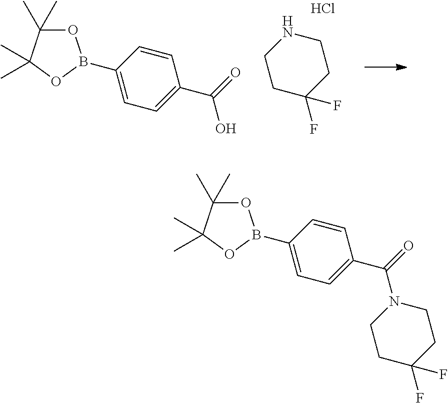

A flask was charged with 4-(4,4,5,5-tetramethyl-[1,3,2]dioxaborolan-2-yl)-benzoic acid (0.50 g, 2.0 mmol), 4,4-difluoropiperidine-HCl (0.381 g, 2.419 mmol), HATU (0.996 g, 2.62 mmol) and TEA (1.05 mL, 8.06 mmol) in DCM (8.06 mL). The mixture was stirred at rt for about 2 h. The mixture was washed with saturated aqueous NaHCO₃ (25 mL) and extracted with DCM (2×20 mL). The organic layers were combined and dried over anhydrous MgSO₄, filtered, and concentrated in vacuo to give crude (4,4-difluoropiperidin-1-yl)(4-(4,4,5,5-tetramethyl-1,3,2-dioxaborolan-2-yl)phenyl)methanone (1.7 g) that was used immediately in the next step without further purification or characterization.

TABLE E.1
Examples prepared from an amine and a carboxylic acid using General
Procedure E

| | | | | Rₜ min | m/z | |
|---|---|---|---|---|---|---|
| | Carboxylic | | | (Table 3, | ESI+ | Syk |
| Amine | Acid | Product | Example # | Method) | (M + H)⁺ | IC₅₀ |
| (3R,5R)-5-(3-(4- methoxyphenyl)- 6H-imidazo[1,5- a]pyrrolo[2,3- e]pyrazin-1- yl)pyrrolidin-3-ol (Preparation #5) | 2-Fluoroacetic acid | 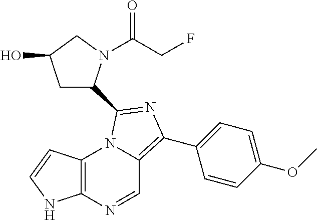 | E.1.1 | 1.56 (a) | 410 | B |
| (3R,5R)-5-(3-(4- (2- methoxyethoxy)- phenyl)-6H- imidazo[1,5- a]pyrrolo[2,3- e]pyrazin-1- yl)pyrrolidin-3-ol (Preparation #6) | 2-Fluoroacetic acid | 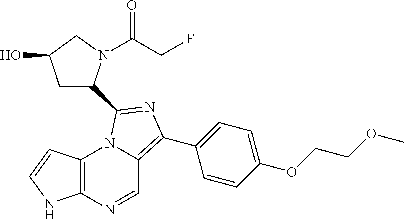 | E.1.2 | 1.52 (a) | 454 | B |

### General Procedure F: Conversion of a (3R,5R)-1-acetyl-5-(3-bromo-6-tosyl-6H-imidazo[1,5-a]pyrrolo[2,3-e]pyrazin-1-yl)pyrrolidin-3-yl acetate to a 1-((2R,4R)-4-hydroxy-2-(6-tosyl-6H-imidazo[1,5-a]pyrrolo[2,3-e]pyrazin-1-yl)pyrrolidin-1-yl)ethanone

To a mixture of a (3R,5R)-1-acetyl-5-(3-bromo-6-tosyl-6H-imidazo[1,5-a]pyrrolo[2,3-e]pyrazin-1-yl)pyrrolidin-3-yl acetate (preferably 1 equiv), a boronic acid or boronate ester (1 to 2 equiv, preferably 1.1 to 1.2 equiv), and an inorganic base (such as, KF, Na₂CO₃, K₂CO₃ or Cs₂CO₃, preferably Cs₂CO₃) (1.1 to 16 equiv, preferably 1.5 to 3 equiv) in a solvent (such as THF, DME, DMF, 1,4-dioxane, DME/water, 1,4-dioxane/water, toluene/EtOH/water, 1,4-dioxane/EtOH/water or water; preferably toluene/EtOH/water or 1,4-dioxane) is added a palladium catalyst (for example Pd₂dba₃, Pd(PPh₃)₄, bis(acetato)triphenylphosphinepalladium(II), polymer-bound FibreCat™ 1032, PdCl₂(dppf), Pd(OAc)₂ or Pd(PPh₃)₂Cl₂; preferably PdCl₂(dppf), Pd(PPh₃)₂Cl₂, Pd(OAc)₂, Pd₂dba₃, 0.01 to 0.20 equiv, preferably 0.02 to 0.1 equiv) and a ligand (for example tricyclohexylphosphine, tri-tert-butyl-phosphine, CgPPh; preferably CgPPh, tricyclohexylphosphine; 0.01-1.0 equiv, preferably 0.16 equiv) is added optionally. The mixture is optionally evacuated and purged with nitrogen after the addition of any reagent. The mixture is then heated at about 40 to 150° C. (preferably about 65 to 120° C.) for about 1 to 48 h (preferably about 2 to 24 h) thermally, or at about 100 to 200° C. (preferably about 100 to 150° C.) for about 5 min to 6 h (preferably about 15 min to 2 h) in a microwave (preferably 2 to 10 min ramp time, 150 to 300 Watts max power, 250 psi max pressure). If the desired product is present, the mixture can be worked up as described in General Procedure A. If the intermediate N-tosyl compound is present, the solvent is optionally removed in vacuo and the residue dissolved in an organic solvent (such as THF, DME, EtOH, 1,4-dioxane or MeOH, preferably 1,4-dioxane or MeOH). An aqueous solution of a base (such as 1 to 6 N; NaOH or KOH; 4 to 20 equiv, preferably 2 to 15 equiv) is added. To the mixture is optionally added a sulfur nucleuophile (such as cystein, tert-butylsulfide, preferably cysteine; 1 to 10 equiv, preferably 2-5 equiv). The mixture is then either heated at about 40 to 110° C. (preferably about 50 to 95° C.) in an oil bath for about 1 to 48 h (preferably about 2 to 16 h) or heated in a microwave at about 80 to 200° C. (preferably 100 to 150° C.) for about 10 to 60 min (preferably 15 to 30 min) (250 psi maximum pressure, 2 to 10 min ramp time, 150 to 300 max watts). The mixture is optionally concentrated in vacuo to give the targeted compound. The mixture is optionally filtered through a media (such as silica gel or Celite®) which is rinsed with an appropriate solvent (such as EtOAc, 1,4-dioxane, THF, MeCN, DCM, Et₂O, MeOH, or EtOH) and then optionally concentrated in vacuo to give a residue. Either the residue or the solution may be optionally partitioned between water and an organic solvent (such as EtOAc, Et₂O or DCM). The organic layer is isolated and may be optionally washed in no particular order with water and/or aqueous solutions containing an acid (such as HCl, AcOH or NH₄Cl) and/or aqueous solutions containing a base (such as NaHCO₃, Na₂CO₃, NaOH, KOH or NH₄OH) and/or aqueous solutions containing an inorganic salt (such as NaCl, Na₂SO₃ or Na₂S₂O₃). The organic solution may then be optionally dried with a drying agent (such as anhydrous MgSO₄ or Na₂SO₄), filtered and concentrated in vacuo to give the targeted compound.

### Illustration of General Procedure F

### Example #F.1: 1-((2R,4R)-2-(3-(4-(ethylamino)phenyl)-6H-imidazo[1,5-a]pyrrolo[2,3-6.]pyrazin-1-yl)-4-hydroxypyrrolidin-1-yl)ethanone

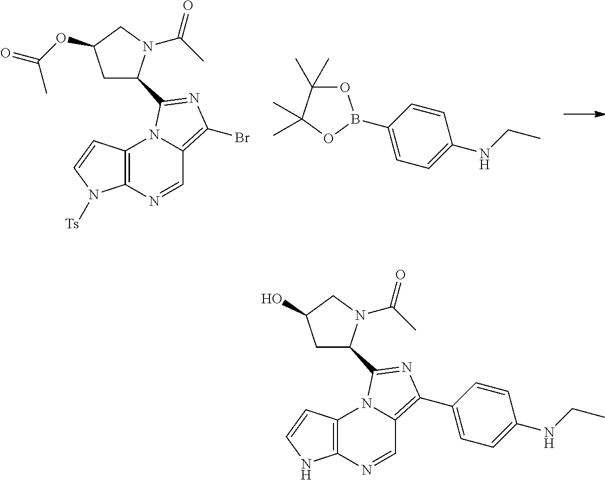

To a flask was added N-ethyl-4-(4,4,5,5-tetramethyl-1,3,2-dioxaborolan-2-yl)aniline (0.165 g, 0.668 mmol, Milestone), (3R,5R)-1-acetyl-5-(3-(4-(ethylamino)phenyl)-6-tosyl-6H-imidazo[1,5-a]pyrrolo[2,3-e]pyrazin-1-yl)pyrrolidin-3-yl acetate (0.401 g, 0.668 mmol, Preparation #1), K₂CO₃ (0.277 g, 2.00 mmol) and PdCl₂(dppf) (0.027 g, 0.033 mmol) 1,4-dioxane (15 mL) and water (5.00 mL). The mixture was stirred overnight at about 120° C. The solvent was removed in vacuo and the residue was purified by Prep-HPLC (Table 3, method j) to afford 1-((2R,4R)-2-(3-(4-(ethylamino)phenyl)-6H-imidazo[1,5-a]pyrrolo[2,3-e]pyrazin-1-yl)-4-hydroxypyrrolidin-1-yl)ethanone (0.180 g, 67%). LC/MS (Table 3, Method i) Rₜ=1.48 min.; MS m/z: 405 (M+H)⁺. Syk IC₅₀=B.

TABLE F.1
Examples prepared from a (3R,5R)-1-acetyl-5-(3-bromo-6-tosyl-6H-
imidazo[1,5-a]pyrrolo[2,3-e]pyrazin-1-yl)pyrrolidin-3-yl acetate with a boronic acid or
boronate using General Procedure F

| (3R,5R)-1-Acetyl-5- | | | | | | |
|---|---|---|---|---|---|---|
| (3-bromo-6-tosyl- | | | | | | |
| 6H-imidazo[1,5- | | | | | | |
| a]pyrrolo[2,3- | | | | | m/z | |
| e]pyrazin-1- | | | | Rₜ min | ESI+ | |
| yl)pyrrolidin-3-yl | Boronic acid or | | Exam- | (Table 3, | (M + | Syk |
| acetate | boronate | Product | ple # | Method) | H)⁺ | IC₅₀ |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | (4,4- Difluoropiperidin- 1-yl)(4-(4,4,5,5- tetramethyl-1,3,2- dioxaborolan-2- yl)phenyl)methanone (Preparation #E.1) | 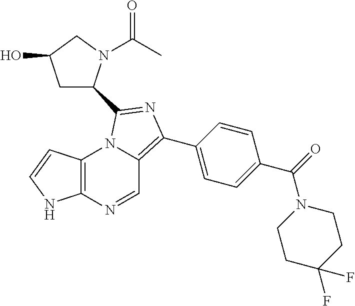 [SMILES: CC(=O)N1C[C@H](O)C[C@@H]1c1nc(-c2ccc(C(=O)N3CCC(F)(F)CC3)cc2)c2cnc3[nH]ccc3n12] | F.1.1 | 1.37 (d) | 509 | D |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | (R)-2-(4-(1- Methoxyethyl)phenyl)- 4,4,5,5- tetramethyl-1,3,2- dioxaborolane (Prepared using B from Preparation #G.1 with 4,4,4′,4′,5,5,5′,5′- octamethyl-2,2′- bi(1,3,2- dioxaborolane)) | 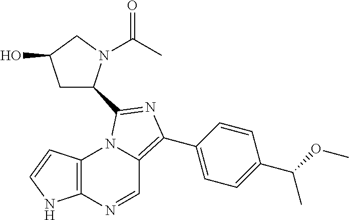 [SMILES: CO[C@H](C)c1ccc(-c2nc([C@H]3C[C@@H](O)CN3C(C)=O)n3c2cnc2[nH]ccc23)cc1] | F.1.2 | 1.56 (a) | 420 | B |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | (R)-2-(4-(1- Methoxyethyl)phenyl)- 4,4,5,5- tetramethyl-1,3,2- dioxaborolane (Prepared using G from (S)-1-(4- bromophenyl)ethanol and B with 4,4,4′,4′,5,5,5′,5′- octamethyl-2,2′- bi(1,3,2- dioxaborolane)) (prepared using B from 1-(4- bromophenyl)cyclo- butanol (Biogene Org) with 4,4,4′,4′,5,5,5′,5′- octamethyl-2,2′- bi(1,3,2- dioxaborolane)) | 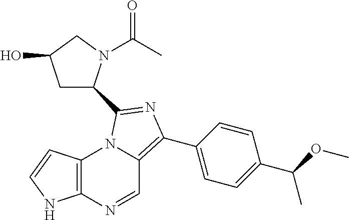 [SMILES: CO[C@@H](C)c1ccc(-c2nc([C@H]3C[C@@H](O)CN3C(C)=O)n3c2cnc2[nH]ccc23)cc1] | F.1.3 | 1.56 (a) | 420 | A |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | (4- Methoxyphenyl)bor- onic acid | 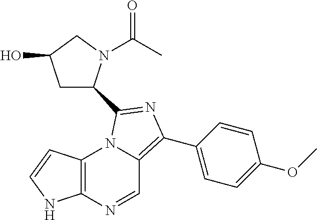 [SMILES: COc1ccc(-c2nc([C@H]3C[C@@H](O)CN3C(C)=O)n3c2cnc2[nH]ccc23)cc1] | F.1.4 | 1.71 (a) | 392 | B |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | (3-Fluoro-4-(2- hydroxypropan-2- yl)phenyl)boronic acid (ChemMaker) | 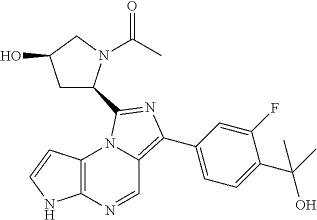 [SMILES: CC(=O)N1C[C@H](O)C[C@@H]1c1nc(-c2ccc(C(C)(C)O)c(F)c2)c2cnc3[nH]ccc3n12] | F.1.5 | 1.47 (a) | 438 | B |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | (2,3- Dihydrobenzo[b][1, 4]dioxin-6- yl)boronic acid | 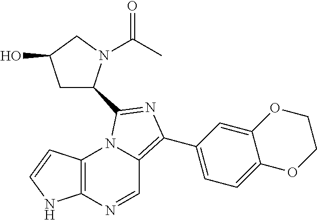 [SMILES: CC(=O)N1C[C@H](O)C[C@@H]1c1nc(-c2ccc3c(c2)OCCO3)c2cnc3[nH]ccc3n12] | F.1.6 | 1.52 (a) | 420 | C |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 2-Methyl-4-(4- (4,4,5,5- tetramethyl-1,3,2- dioxaborolan-2- yl)phenyl)morpholine (Prepared using C with 1,4- dibromobenzene and 2-methyl morpholine (Anichem) and B with 4,4,5,5- tetramethyl- [1,3,2]dioxaborolane) | 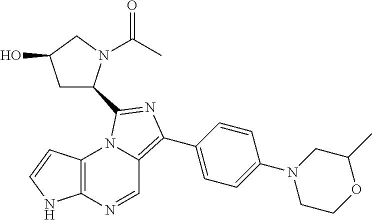 [SMILES: CC(=O)N1C[C@H](O)C[C@@H]1c1nc(-c2ccc(N3CCOC(C)C3)cc2)c2cnc3[nH]ccc3n12] | F.1.7 | 1.67 (a) | 461 | A |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 4,4-Difluoro-1-(4- (4,4,5,5- tetramethyl-1,3,2- dioxaborolan-2- yl)phenyl)piperidine morpholine (Prepared using C with 1,4- dibromobenzene and 4,4- difluoropiperidine and B with 4,4,5,5- tetramethyl- [1,3,2]dioxaborolane) | 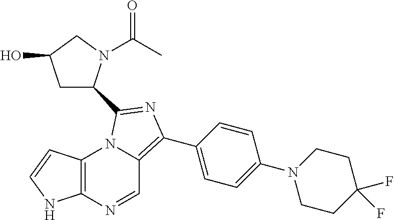 [SMILES: CC(=O)N1C[C@H](O)C[C@@H]1c1nc(-c2ccc(N3CCC(F)(F)CC3)cc2)c2cnc3[nH]ccc3n12] | F.1.8 | 1.82 (a) | 481 | A |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 3,3-Difluoro-1-(4- (4,4,5,5- tetramethyl-1,3,2- dioxaborolan-2- yl)phenyl)azetidine (Prepared using B from Preparation #C.1 with 4,4,5,5- tetramethyl- [1,3,2]dioxaborolane) | 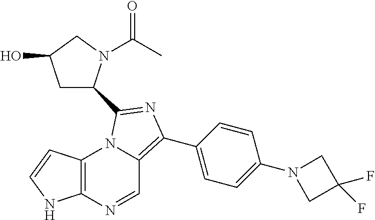 [SMILES: CC(=O)N1C[C@H](O)C[C@@H]1c1nc(-c2ccc(N3CC(F)(F)C3)cc2)c2cnc3[nH]ccc3n12] | F.1.9 | 1.73 (a) | 453 | A |
| (3R,5R)-1-Acetyl-5- (3-bromo-8-chloro- 6-tosyl-6H- imidazo[1,5- a]pyrrolo[2,3- e]pyrazin-1- yl)pyrrolidin-3-yl acetate (Preparation #3) | 4- Morpholinophenyl- boronic acid (Combi Blocks) | 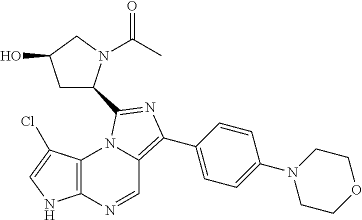 [SMILES: CC(=O)N1C[C@H](O)C[C@@H]1c1nc(-c2ccc(N3CCOCC3)cc2)c2cnc3[nH]cc(Cl)c3n12] | F.1.10 | 1.61 (a) | 481 | A |
| (3R,5R)-1-Acetyl-5- (3-bromo-8-chloro- 6-tosyl-6H- imidazo[1,5- a]pyrrolo[2,3- e]pyrazin-1- yl)pyrrolidin-3-yl acetate (Preparation #3) | 4- Ethoxyphenylboronic acid | 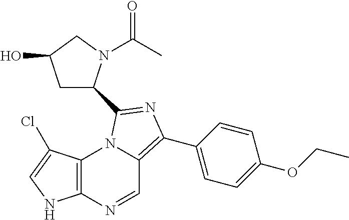 [SMILES: CCOc1ccc(-c2nc([C@H]3C[C@@H](O)CN3C(C)=O)n3c2cnc2[nH]cc(Cl)c23)cc1] | F.1.11 | 1.83 (a) | 440 | B |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 4-(2- Morpholinoethoxy)- phenylboronic acid | 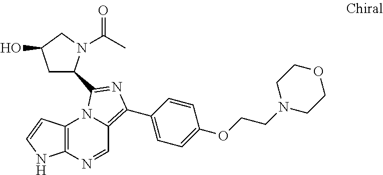 [SMILES: CC(=O)N1C[C@H](O)C[C@@H]1c1nc(-c2ccc(OCCN3CCOCC3)cc2)c2cnc3[nH]ccc3n12] | F.1.12 | 1.45 (b) | 491 | B |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 1-(4-(4,4,5,5- Tetramethyl-1,3,2- dioxaborolan-2- yl)phenyl)piperidine | 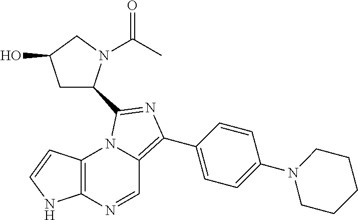 [SMILES: CC(=O)N1C[C@H](O)C[C@@H]1c1nc(-c2ccc(N3CCCCC3)cc2)c2cnc3[nH]ccc3n12] | F.1.1.13 | 1.91 (a) | 445 | A |
| (3R,5R)-1-acetyl-5- (3-bromo-8-methyl- 6-tosyl-6H- imidazo[1,5- a]pyrrolo[2,3- e]pyrazin-1- yl)pyrrolidin-3-yl acetate (Preparation #2) | 4- Methoxyphenyl- boronic acid (Small Molecules, Inc.) | 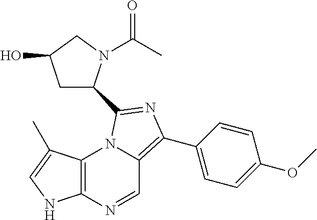 [SMILES: COc1ccc(-c2nc([C@H]3C[C@@H](O)CN3C(C)=O)n3c2cnc2[nH]cc(C)c23)cc1] | F.1.14 | 1.75 (a) | 406 | C |
| 3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 2-(4-(4,4,5,5- Tetramethyl-1,3,2- dioxaborolan-2- yl)phenyl)propan- 2-ol (Novel Chemical Solutions, Inc.) | 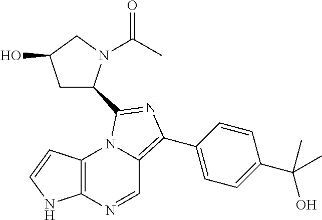 [SMILES: CC(=O)N1C[C@H](O)C[C@@H]1c1nc(-c2ccc(C(C)(C)O)cc2)c2cnc3[nH]ccc3n12] | F.1.15 | 1.63 (a) | 420 | A |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 4-(2-Cyanopropan- 2-yl)phenylboronic acid | 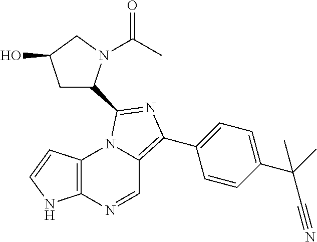 | F.1.16 | 1.64 (a) | 429 | A |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 4- Morpholinophenyl- boronic acid | 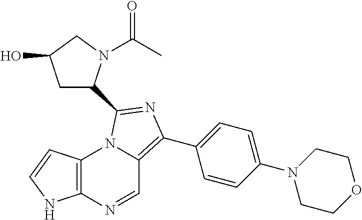 | F.1.17 | 1.67 (a) | 447 | A |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 4,4,5,5- Tetramethyl-2-(4- (tetrahydro-2H- pyran-4-yl)phenyl)- 1,3,2- dioxaborolane (prepared using B from 4-(4- bromophenyl)tetra- hydro-2H-pyran with 4,4,4′,4′,5,5,5′,5′- octamethyl-2,2′- bi(1,3,2- dioxaborolane) | 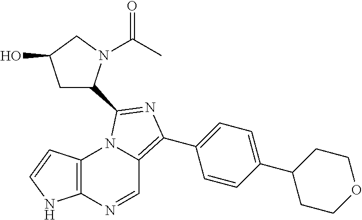 | F.1.18 | 1.72 (a) | 445 | A |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 2-(Chroman-6-yl)- 4,4,5,5-tetramethyl- 1,3,2- dioxaborolane (prepared using B from 6-bromochroman with 4,4,4′,4′,5,5,5′,5′- octamethyl-2,2′- bi(1,3,2- dioxaborolane)) | 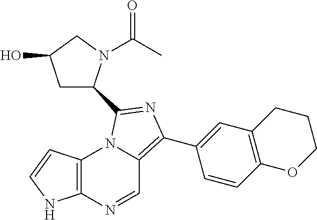 [SMILES: CC(=O)N1C[C@H](O)C[C@@H]1c1nc(-c2ccc3c(c2)CCCO3)c2cnc3[nH]ccc3n12] | F.1.19 | 1.65 (a) | 406 | B |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 3,4- Dimethoxyphenyl- boronic acid | 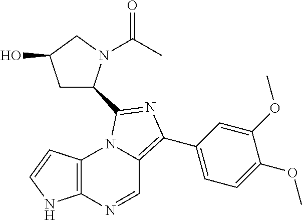 [SMILES: COc1ccc(-c2nc([C@H]3C[C@@H](O)CN3C(C)=O)n3c2cnc2[nH]ccc23)cc1OC] | F.1.20 | 1.64 (a) | 422 | A |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 4-(2- Methoxyethoxy)- phenyl- boronic acid | 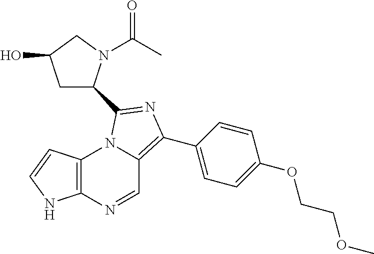 [SMILES: COCCOc1ccc(-c2nc([C@H]3C[C@@H](O)CN3C(C)=O)n3c2cnc2[nH]ccc23)cc1] | F.1.21 | 1.67 (a) | 436 | B |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 4-Methoxy-3- methylphenylboronic acid | 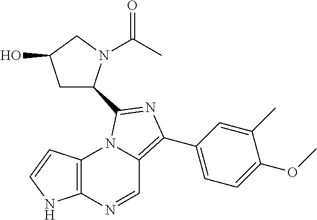 [SMILES: COc1ccc(-c2nc([C@H]3C[C@@H](O)CN3C(C)=O)n3c2cnc2[nH]ccc23)cc1C] | F.1.22 | 1.66 (a) | 406 | B |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 4- Isopropoxyphenyl- boronic acid | 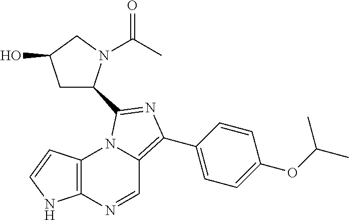 [SMILES: CC(=O)N1C[C@H](O)C[C@@H]1c1nc(-c2ccc(OC(C)C)cc2)c2cnc3[nH]ccc3n12] | F.1.23 | 1.77 (a) | 420 | B |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | Benzo[d][1,3]dioxol- 5-ylboronic acid | 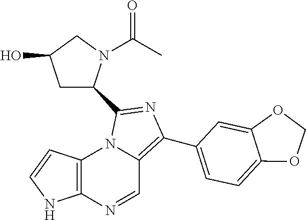 [SMILES: CC(=O)N1C[C@H](O)C[C@@H]1c1nc(-c2ccc3c(c2)OCO3)c2cnc3[nH]ccc3n12] | F.1.24 | 1.49 (a) | 407 | D |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 4- (Trifluoromethoxy) phenylboronic acid | 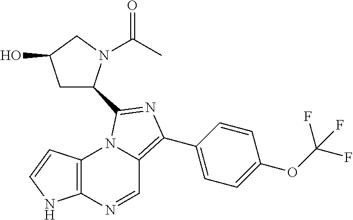 [SMILES: CC(=O)N1C[C@H](O)C[C@@H]1c1nc(-c2ccc(OC(F)(F)F)cc2)c2cnc3[nH]ccc3n12] | F.1.25 | 1.87 (a) | 446 | |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 3-Fluoro-4- methoxyphenyl- boronic acid | 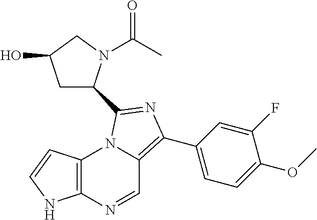 [SMILES: COc1ccc(-c2nc([C@H]3C[C@@H](O)CN3C(C)=O)n3c2cnc2[nH]ccc23)cc1F] | F.1.26 | 1.55 (a) | 410 | C |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 1-(4-(4,4,5,5- Tetramethyl-1,3,2- dioxaborolan-2- yl)phenyl)- cyclobutanol | 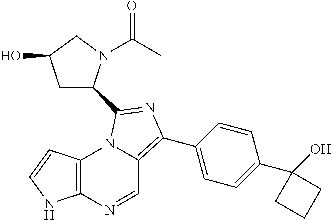 [SMILES: CC(=O)N1C[C@H](O)C[C@@H]1c1nc(-c2ccc(C3(O)CCC3)cc2)c2cnc3[nH]ccc3n12] | F.1.27 | 1.44 (a) | 432 | B |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 4-Ethoxy-3- methylphenyl- boronic acid | 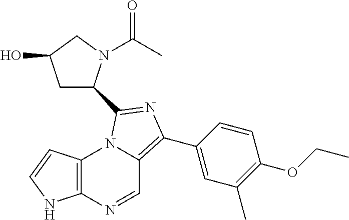 [SMILES: CCOc1ccc(-c2nc([C@H]3C[C@@H](O)CN3C(C)=O)n3c2cnc2[nH]ccc23)cc1C] | F.1.28 | 1.84 (a) | 420 | A |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 6-(4,4,5,5- Tetramethyl-1,3,2- dioxaborolan-2-yl)- 3,4-dihydro-2H- benzo[b][1,4]oxazine (prepared using B from 6-bromo-3,4- dihydro-2H- benzo[b][1,4]oxazine (J & W Pharm) with 4,4,4′,4′,5,5,5′,5′- octamethyl-2,2′- bi(1,3,2- dioxaborolane)) | 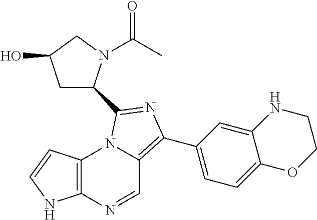 [SMILES: CC(=O)N1C[C@H](O)C[C@@H]1c1nc(-c2ccc3c(c2)NCCO3)c2cnc3[nH]ccc3n12] | F.1.29 | 1.42 (a) | 419 | B |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | 7-(4,4,5,5- Tetramethyl-1,3,2- dioxaborolan-2-yl)- 1,2,3,4- tetrahydroquinoline (Small Molecules Inc.) | 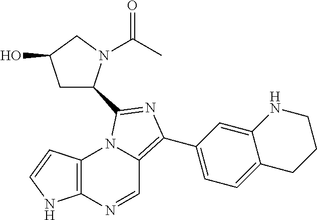 [SMILES: CC(=O)N1C[C@H](O)C[C@@H]1c1nc(-c2ccc3c(c2)NCCC3)c2cnc3[nH]ccc3n12] | F.1.30 | 1.61 (a) | 417 | B |
| (3R,5R)-1-Acetyl-5- (3-bromo-6-tosyl- 6H-imidazo[1,5- a]pyrrolo[2,3- e]pyrazin-1- yl)pyrrolidin-3-yl acetate (Preparation #1) | (4- Cyclopropoxy- phenyl)boronic acid (Ark Pharm) | 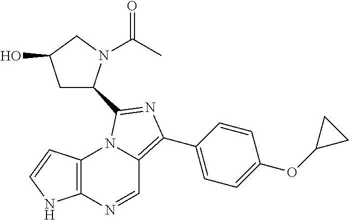 [SMILES: CC(=O)N1C[C@H](O)C[C@@H]1c1nc(-c2ccc(OC3CC3)cc2)c2cnc3[nH]ccc3n12] | F.1.31 | 1.71 (a) | 418 | B |

General Procedure G: Formation of a Methyl Ether from an Alcohol

To a flask is added an alcohol (1 equiv) and an organic solvent (such as NMP, DMF, THF, Et₂O, or 1,2-DME, preferably DMF) and the temperature of the solution is maintained at about −30° C. to 45° C. (preferably about −10 to 10° C.) for about 1 to 60 min (preferably about 10 min). To this mixture is added a base (such as NaH, KH, Cs₂CO₃, or K₂CO₃, preferably NaH; 1 to 10 equiv, preferably 1 to 2 equiv) and the solution is stirred at about −30 to 45° C. (preferably about −10 to 10° C.) for about 1 min to 2 h (preferably about 20 min). To the mixture is added a methylating reagent (such as MeI, MeBr, MeCl, MeOTf, or dimethylsulfate, preferably MeI; 1 to 20 equiv, preferably 1 to 1.5 equiv) and the solution is stirred at about −10 to 85° C. (preferably about 10 to 40° C.) for about 10 min to 4 h (preferably about 30 min). The mixture is treated with an aqueous acid solution (such as HCl, AcOH, or phosphoric acid, preferably HCl) and extracted with an organic solvent (such as EtOAc, DCM, or Et₂O, preferably EtOAc). The organic layer is isolated and may be optionally washed in no particular order with water and/or aqueous solutions containing an acid (such as HCl, AcOH or NH₄Cl) and/or aqueous solutions containing a base (such as NaHCO₃, Na₂CO₃, NaOH, KOH or NH₄OH) and/or aqueous solutions containing an inorganic salt (such as NaCl, Na₂SO₃ or Na₂S₂O₃). The organic solution may then be optionally dried with a drying agent (such as anhydrous MgSO₄ or Na₂SO₄), filtered and concentrated in vacuo to give the targeted compound.

### Illustration of General Procedure G

### Preparation #G.1: (R)-1-bromo-4-(1-methoxyethyl)benzene

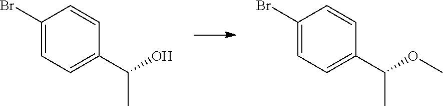

A flask equipped was charged with (R)-1-(4-bromophenyl)ethanol (0.550 g, 2.74 mmol) in DMF (5.5 ml). The mixture was cooled at about 0° C. for about 10 min and then NaH (60% dispersion in mineral oil, 0.164 g, 4.10 mmol) was added portionwise. The mixture was allowed to stir at about 0° C. for about 20 min and then MeI (0.205 ml, 3.28 mmol) was added dropwise. The resulting mixture was allowed to stir at rt for about 30 min. The mixture was diluted with aqueous HCl solution (1.0 N, 50 mL) and extracted with EtOAc (2×50 mL). The combined organic phases were dried over anhydrous MgSO₄, filtered and concentrated to give crude (R)-1-bromo-4-(1-methoxyethyl)benzene (0.645 g, 110%) that was used in the next step without further purification: LC/MS (Table 3, Method a) Rₜ=2.54 min MS m/z no parent ion peak (M+H)⁺.

### General Procedure H: Mitsunobu Reaction of an Alcohol to Form an Ether

To a mixture of an azodicarboxylate, such as DIAD or DEAD (preferably DIAD) (1 to 3 equiv, preferably 2.5 equiv), a phosphine, such as triphenylphosphine or tributylphosphine (preferably triphenylphosphine; 1 to 3 equiv, preferably 2.5 equiv), and an alcohol (1 to 5 equiv, preferably 2 equiv) in a solvent (such as THF, DMF, or 1,4-dioxane; preferably THF) is added an alcohol (such as (3R,5R)-1-acetyl-5-(3-(4-hydroxyphenyl)-6-tosyl-6H-imidazo[1,5-a]pyrrolo[2,3-e]pyrazin-1-yl)pyrrolidin-3-yl acetate, 1 equiv). The mixture is stirred at about 0 to 60° C. (preferably about 15 to 40° C.) for about 1 to 24 h (preferably about 8 to 16 h). If necessary, the mixture is allowed to cool to rt and is worked up using one of the following methods. Method 1. The mixture is concentrated in vacuo and optionally purified using one or more of the purification methods described above to give the desired compound. Method 2. The reaction mixture may be diluted with an organic solvent (such as DCM or EtOAc). The layers are separated, the organic solution is optionally washed with water and/or brine, dried over anhydrous MgSO₄ or Na₂SO₄, filtered, and the solvent is removed in vacuo to give the desired compound.

### Illustration of General Procedure H

### Preparation #H.1: 1-((2R,4R)-4-hydroxy-2-(3-(4-((3-methyloxetan-3-yl)methoxy)phenyl)-6H-imidazo[1,5-a]pyrrolo[2,3-e]pyrazin-1-yl)pyrrolidin-1-yl)ethanone

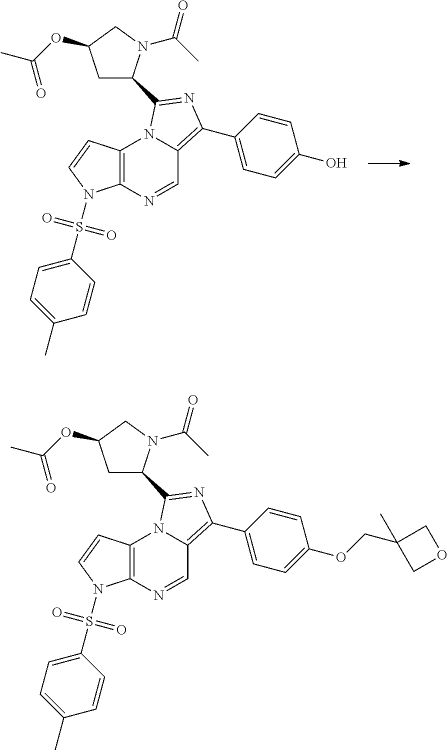 [SMILES: CC(=O)O[C@@H]1C[C@H](c2nc(-c3ccc(O)cc3)c3cnc4c(ccn4S(=O)(=O)c4ccc(C)cc4)n23)N(C(C)=O)C1 ; CC(=O)O[C@@H]1C[C@H](c2nc(-c3ccc(OCC4(C)COC4)cc3)c3cnc4c(ccn4S(=O)(=O)c4ccc(C)cc4)n23)N(C(C)=O)C1]

To a flask was added DIAD (0.12 mL, 0.654 mmol) and triphenylphosphine (0.17 g, 0.654 mmol) in THF (1.3 mL). The mixture was stirred for about 2 min at rt followed by the addition of (3R,5R)-1-acetyl-5-(3-(4-hydroxyphenyl)-6-tosyl-6H-imidazo[1,5-a]pyrrolo[2,3-e]pyrazin-1-yl)pyrrolidin-3-yl acetate (0.150 g, 0.261 mmol, Preparation #A.1) and 3-methyl-3-oxetanemethanol (0.052 g, 0.523 mmol). The mixture was stirred at rt for about 16 h. The solvent was concentrated in vacuo. The crude residue was purified by flash chromatography (silicagel, 0 to 5% MeOH in DCM) to give (3R,5R)-1-acetyl-5-(3-(4-((3-methyloxetan-3-yl)methoxy)phenyl)-6-tosyl-6H-imidazo[1,5-a]pyrrolo[2,3-e]pyrazin-1-yl)pyrrolidin-3-yl acetate (0.035 g, 29%). LC/MS (Table 3, Method c) Rₜ=0.74 min; MS m/z 658 (M+H)⁺.

### Example #1

### 1-((2R,4R)-4-Hydroxy-2-(3-(4-(methylamino)phenyl)-6H-imidazo[1,5-a]pyrrolo[2,3-e]pyrazin-1-yl)pyrrolidin-1-yl)ethanone

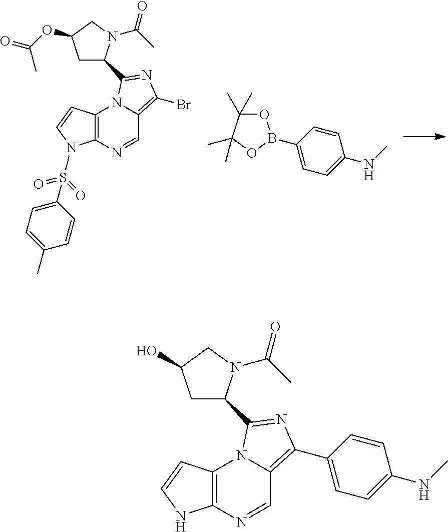

A mixture of (3R,5R)-1-acetyl-5-(3-bromo-6-tosyl-6H-imidazo[1,5-a]pyrrolo[2,3-e]pyrazin-1-yl)pyrrolidin-3-yl acetate (6.0 g, 10.0 mmol, Preparation #1), N-methyl-4-(4,4,5,5-tetramethyl-1,3,2-dioxaborolan-2-yl)aniline (CombiBlocks) (3.74 g, 16.1 mmol) and K₂CO₃ (4.44 g, 32.1 mmol) in 1,4-dioxane (143 mL) and water (35 mL) and degassed by applying vacuum and backfilling with nitrogen 3 times. To the mixture was added PdCl₂(dppf) (0.783 g, 1.07 mmol) and the degassing procedure was repeated. The mixture was heated to about 100° C. for about 16 h. To the mixture was added aqueous NaOH (1.0 N, 64.2 mL, 64.2 mmol) and the mixture was stirred at about 90° C. for about 2 h. Additional aqueous NaOH (1.0 N, 15 mL, 15 mmol) was added and the mixture was stirred at about 90° C. for about 2 hours. The mixture was concentrated in vacuo and the resulting aqueous phase was treated with saturated aqueous NH₄Cl (250 mL). The aqueous phase was extracted with DCM (3×300 mL) and the combined organic layers were washed with brine (400 mL), dried over anhydrous MgSO₄, filtered and concentrated in vacuo. The residue was suspended in MeOH (120 mL) and the precipitate was collected by filtration and dried in vacuo to yield a solid. The solid was recrystallized from MeOH (250 mL) to yield a solid. The mother liquor was concentrated in vacuo and the residue was recrystallized from MeOH (120 mL). The mother liquor was concentrated in vacuo and purified by flash chromatography (silica gel; DCM/MeOH 1:0 to 92:8) to give a solid. The three solids were combined and recrystallized from MeOH (175 mL) to give 1-((2R,4R)-4-Hydroxy-2-(3-(4-(methylamino)phenyl)-6H-imidazo[1,5-a]pyrrolo[2,3-e]pyrazin-1-yl)pyrrolidin-1-yl)ethanone (3.06 g; 37%). LC/MS (Table 3, Method b) Rₜ=1.42 min; MS m/z 391 (M+H)⁺. Syk IC₅₀=B.

### Example #2

### 1-((2R,4R)-2-(3-(4-((R)-1-Cyclopropylethoxy)phenyl)-6H-imidazo[1,5-a]pyrrolo[2,3-e]pyrazin-1-yl)-4-hydroxypyrrolidin-1-yl)ethanone

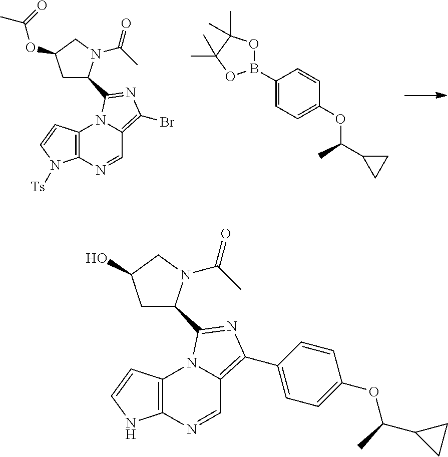 [SMILES: CC(=O)O[C@@H]1C[C@H](c2nc(Br)c3cnc4c(ccn4S(=O)(=O)c4ccc(C)cc4)n23)N(C(C)=O)C1 ; C[C@@H](Oc1ccc(B2OC(C)(C)C(C)(C)O2)cc1)C1CC1 ; CC(=O)N1C[C@H](O)C[C@@H]1c1nc(-c2ccc(O[C@H](C)C3CC3)cc2)c2cnc3[nH]ccc3n12]

A racemic mixture of 2-(4-(1-cyclopropylethoxy)phenyl)-4,4,5,5-tetramethyl-1,3,2-dioxaborolane (3.65 g, prepared using H using 4-bromophenol and 1-cyclopropylethanol and B with 4,4,4′,4′,5,5,5′,5′-octamethyl-2,2′-bi(1,3,2-dioxaborolane)) was separated by preparative chiral chromatography (Table 3, Method h) and the first eluting peak was isolated and concentrated to give (R)-2-(4-(1-cyclopropylethoxy)phenyl)-4,4,5,5-tetramethyl-1,3,2-dioxaborolane as a white solid (1.22 g, 99% ee, absolute stereochemistry arbitrarily assigned). A 5 mL microwave reaction vial equipped with pressure-releasing septa cap was charged with (3R,5R)-1-acetyl-5-(3-bromo-6-tosyl-6H-imidazo[1,5-a]pyrrolo[2,3-e]pyrazin-1-yl)pyrrolidin-3-yl acetate (0.400 g, 0.714 mmol, Preparation #1), (R)-2-(4-(1-cyclopropylethoxy)phenyl)-4,4,5,5-tetramethyl-1,3,2-dioxaborolane (0.247 g, 0.857 mmol), SiliaCat® diphenylphosphinopropane-Pd (0.297 g, 0.071 mmol), evacuated and filled with nitrogen three times, and then 1,4-dioxane (1.55 mL) and EtOH (1.55 mL) were each added. A solution of Cs₂CO₃ (0.698 g, 2.14 mmol) in water (0.465 mL) was added. The mixture was heated in a microwave at about 150° C. (250 psi maximum pressure, 10 min ramp, 150 max watts) for about 1 h. To the mixture was added aqueous sodium hydroxide solution (1.0 N, 2.14 mL, 2.14 mmol) and the mixture was heated in a microwave at about 150° C. (250 psi max pressure, 10 min ramp, 150 max watts) for about 1 h. The mixture was passed through Celite® and dried over anhydrous MgSO₄. The crude material was purified via silica gel chromatography (0 to 10% MeOH in DCM) to afford 1-((2R,4R)-2-(3-(4-((R)-1-cyclopropylethoxy)phenyl)-6H-imidazo[1,5-a]pyrrolo[2,3-e]pyrazin-1-yl)-4-hydroxypyrrolidin-1-yl)ethanone (0.120 g, 38%): LC/MS (Table 3, Method a) Rₜ=1.90 min; MS m/z: 446 (M+H)⁺. Syk IC₅₀=C.

### Example #3

### 1-((2R,4R)-4-Hydroxy-2-(3-(5-methoxythiophen-2-yl)-6H-imidazo[1,5-a]pyrrolo[2,3-e]pyrazin-1-yl)pyrrolidin-1-yl)ethanone

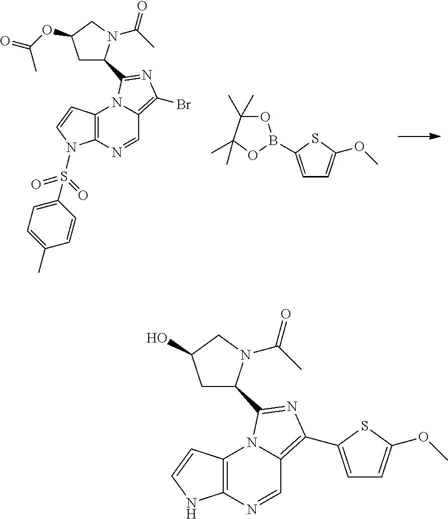

A flask was charged with (3R,5R)-1-acetyl-5-(3-bromo-6-tosyl-6H-imidazo[1,5-a]pyrrolo[2,3-e]pyrazin-1-yl)pyrrolidin-3-yl acetate (0.100 g, 0.178 mmol, Preparation #1), 2-(5-methoxythiophen-2-yl)-4,4,5,5-tetramethyl-1,3,2-dioxaborolane (0.086 g, 0.357 mmol, Peptech) and Cs₂CO₃ (0.145 g, 0.446 mmol) in a mixture of 1,4-dioxane (1.3 mL) and water (0.35 mL). The mixture was degassed by applying vacuum and backfilling with nitrogen 3 times. To the mixture was added PdCl₂(PPh₃)₂ (0.008 g, 0.012 mmol) and the degassing procedure was repeated. The mixture was heated to about 75° C. for about 1 h. To the mixture was added aqueous NaOH (1.0 N, 0.892 mL, 0.892 mmol) and the mixture was stirred at about 75° C. for about 1 h. The mixture was cooled to rt and partitioned between water and a solution of 10% IPA in DCM (10 mL each). The aqueous phase was extracted with DCM (3×10 mL) and the combined organic layers were washed with brine (30 mL), dried over anhydrous MgSO₄, filtered, and concentrated in vacuo. The crude residue was purified by flash chromatography (silica gel; 0 to 8% MeOH in DCM) to give 1-((2R,4R)-4-Hydroxy-2-(3-(5-methoxythiophen-2-yl)-6H-imidazo[1,5-e]pyrrolo[2,3-e]pyrazin-1-yl)pyrrolidin-1-yl)ethanone (0.06 g; 85%). LC/MS (Table 3, Method a) Rₜ=1.53 min; MS m/z 398 (M+H)⁺. Syk IC₅₀=B.

### Example #4

### 1-((2R,4R)-2-(3-(2-(tert-Butyl)thiazol-5-yl)-6H-imidazo[1,5-a]pyrrolo[2,3-e]pyrazin-1-yl)-4-hydroxypyrrolidin-1-yl)ethanone

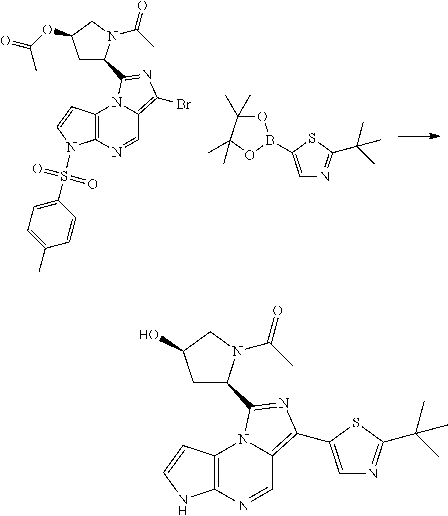 [SMILES: CC(=O)O[C@@H]1C[C@H](c2nc(Br)c3cnc4c(ccn4S(=O)(=O)c4ccc(C)cc4)n23)N(C(C)=O)C1 ; CC(C)(C)c1ncc(B2OC(C)(C)C(C)(C)O2)s1 ; CC(=O)N1C[C@H](O)C[C@@H]1c1nc(-c2cnc(C(C)(C)C)s2)c2cnc3[nH]ccc3n12]

A microwave vial was charged with (3R,5R)-1-acetyl-5-(3-bromo-6-tosyl-6H-imidazo[1,5-c]pyrrolo[2,3-e]pyrazin-1-yl)pyrrolidin-3-yl acetate (0.400 g, 0.714 mmol, Preparation #1), 2-(5-methoxythiophen-2-yl)-4,4,5,5-tetramethyl-1,3,2-dioxaborolane (0.477 g, 1.74 mmol, Combi Phos), aqueous Na₂CO₃ (2 N, 0.145 mL, 0.446 mmol), and PdCl₂(dppf) (0.058 g, 0.071 mmol) in DME (3 mL). The mixture was irradiated in a microwave and heated to about 120° C. (250 psi maximum pressure, 10 min ramp, 300 max watts) for about 15 min. The solvent was concentrated in vacuo and the residue was taken up in 1,4-dioxane (3 mL) followed by the addition of aqueous NaOH (1 N, 3.57 mL, 3.57 mmol). The mixture was heated to about 75° C. for about 1 h. The mixture was cooled to rt and partitioned between water (10 mL) and a solution of 10% IPA in DCM (10 mL). The aqueous phase was extracted with DCM (3×10 mL) and the combined organic layers were dried over anhydrous MgSO₄, filtered, and concentrated in vacuo. The crude residue was purified by flash chromatography (silica gel; 0 to 50% acetone in DCM) to give 1-((2R,4R)-2-(3-(2-(tert-butyl)thiazol-5-yl)-6H-imidazo[1,5-a]pyrrolo[2,3-e]pyrazin-1-yl)-4-hydroxypyrrolidin-1-yl)ethanone (0.201 g; 66%). LC/MS (Table 3, Method a) Rₜ=1.64 min; MS m/z 425 (M+H)⁺. Syk IC₅₀=C.

### Example #5

### 1-((2R,4R)-4-Hydroxy-2-(3-(4-((S)-2-methylmorpholino)phenyl)-6H-imidazo[1,5-a]pyrrolo[2,3-e]pyrazin-1-yl)pyrrolidin-1-yl)ethanone and 1-((2R,4R)-4-hydroxy-2-(3-(4-((R)-2-methylmorpholino)phenyl)-6H-imidazo[1,5-a]pyrrolo[2,3-e]pyrazin-1-yl)pyrrolidin-1-yl)ethanone

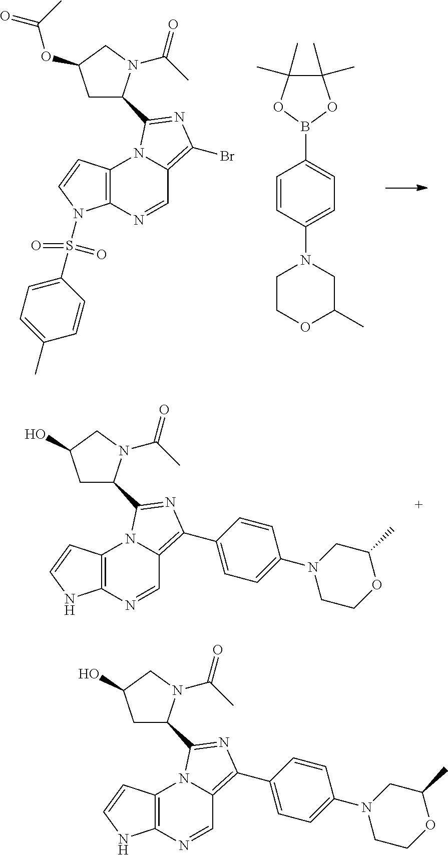

A microwave vial was charged with (3R,5R)-1-acetyl-5-(3-bromo-6-tosyl-6H-imidazo[1,5-a]pyrrolo[2,3-e]pyrazin-1-yl)pyrrolidin-3-yl acetate (0.525 g, 0.937 mmol, Preparation #1), 2-methyl-4-(4-(4,4,5,5-tetramethyl-1,3,2-dioxaborolan-2-yl)phenyl)morpholine (0.312 g, 1.030 mmol, prepared using C from 1,4-dibromobenzene and 2-methylmorpholine and B using 4,4,5,5-tetramethyl-[1,3,2]dioxaborolane), aqueous Na₂CO₃ (2 N, 0.14 mL, 2.81 mmol), and PdCl₂(dppf) (0.077 g, 0.094 mmol) in DME (3.75 mL). The mixture was irradiated in a microwave and heated to about 120° C. (250 psi maximum pressure, 10 min ramp, 300 max watts) for about 15 min. The solvent was concentrated to dryness and the remaining residue was taken up in 1,4-dioxane (3.75 mL) followed by the addition of aqueous NaOH (1 N, 4.6 mL, 4.6 mmol). The mixture was heated to about 75° C. for about 16 h. The mixture was cooled to rt and partitioned between water (10 mL) and a solution of 10% IPA in DCM (10 mL). The aqueous phase was extracted with DCM (3×10 mL) and the combined organic layers were dried over anhydrous MgSO₄, filtered, and concentrated in vacuo. The crude residue was purified by flash chromatography (silica gel; 0 to 10% MeOH in EtOAc) to give (3R,5R)-1-acetyl-5-(3-(4-(2-methylmorpholino)phenyl)-6-tosyl-6H-imidazo[1,5-a]pyrrolo[2,3-e]pyrazin-1-yl)pyrrolidin-3-yl acetate (0.185 g; 30%) as a mixture of diastereomers. LC/MS (Table 3, Method a) Rₜ=1.69 min; MS m/z 461 (M+H)⁺. The diasteromers were resolved using SFC prep (Table 3, Method f) to give 1-((2R,4R)-4-hydroxy-2-(3-(4-((S)-2-methylmorpholino)phenyl)-6H-imidazo[1,5-a]pyrrolo[2,3-e]pyrazin-1-yl)pyrrolidin-1-yl)ethanone (0.037 g, 9%), LC/MS (Table 3, Method g) Rₜ=6.0 min; MS m/z 461 (M+H)⁺. Syk IC₅₀=A. and 1-((2R,4R)-4-hydroxy-2-(3-(4-((R)-2-methylmorpholino)phenyl)-6H-imidazo[ 1,5-a]pyrrolo[2,3-e]pyrazin-1-yl)pyrrolidin-1-yl)ethanone (0.034 g, 8%). LC/MS (Table 3, Method g) Rt=6.27 min; MS m/z 461 (M+H)⁺. Syk IC₅₀=A.

### Example #6

### (2R,4R)-2-(3-(2,3-Dihydrobenzo[b][1,4]dioxin-6-yl)-6H-imidazo[1,5-a]pyrrolo[2,3-e]pyrazin-1-yl)-4-hydroxypyrrolidine-1-carboxamide

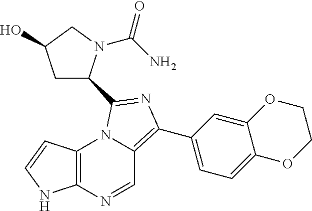 [SMILES: NC(=O)N1C[C@H](O)C[C@@H]1c1nc(-c2ccc3c(c2)OCCO3)c2cnc3[nH]ccc3n12]

### Step A: (3R,5R)-1-Acetyl-5-(3-(2,3-dihydrobenzo[b][1,4]dioxin-6-yl)-6-tosyl-6H-imidazo[1,5-a]pyrrolo[2,3-e]pyrazin-1-yl)pyrrolidin-3-yl acetate

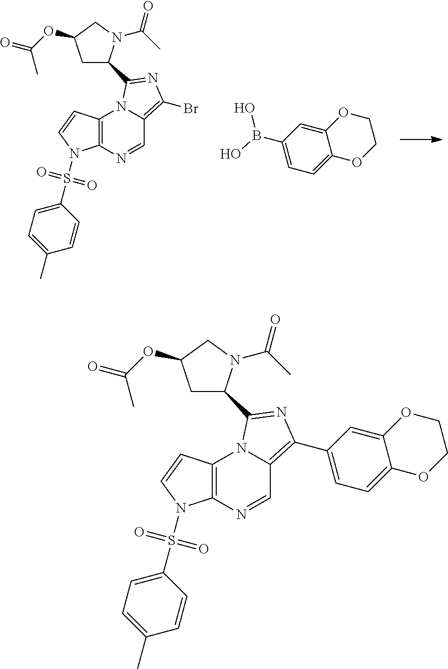 [SMILES: CC(=O)O[C@@H]1C[C@H](c2nc(Br)c3cnc4c(ccn4S(=O)(=O)c4ccc(C)cc4)n23)N(C(C)=O)C1 ; OB(O)c1ccc2c(c1)OCCO2 ; CC(=O)O[C@@H]1C[C@H](c2nc(-c3ccc4c(c3)OCCO4)c3cnc4c(ccn4S(=O)(=O)c4ccc(C)cc4)n23)N(C(C)=O)C1]

A flask was charged with (3R,5R)-1-acetyl-5-(3-bromo-6-tosyl-6H-imidazo[1,5-a]pyrrolo[2,3-e]pyrazin-1-yl)pyrrolidin-3-yl acetate (0.300 g, 0.535 mmol, Preparation #1), chroman-6-ylboronic acid (0.114 g, 0.642 mmol, Enamine), Cs₂CO₃ (0.436 g, 1.34 mmol) and a mixture of 1,4-dioxane (2 mL) in water (0.5 mL). The mixture was degassed by applying vacuum and backfilling with nitrogen 3 times. To the mixture was added PdCl₂(PPh₃)₂ (0.037 g, 0.054 mmol) and the degassing procedure was repeated. The mixture was heated to about 75° C. for about 2 h. The mixture was cooled to rt and partitioned between water (10 mL) and DCM (10 mL). The aqueous phase was extracted with DCM (3×10 mL) and the combined organic layers were dried over anhydrous MgSO₄, filtered, and concentrated in vacuo. The crude residue was purified by flash chromatography (silica gel; 0 to 50% acetone in DCM) to give (3R,5R)-1-acetyl-5-(3-(chroman-6-yl)-6-tosyl-6H-imidazo[1,5-a]pyrrolo[2,3-e]pyrazin-1-yl)pyrrolidin-3-yl acetate (0.272 g, 83%), LC/MS (Table 3, Method a) Rₜ=2.47 min; MS m/z 614 (M+H)⁺.

### Step B: (3R,5R)-5-(3-(Chroman-6-yl)-6-tosyl-6H-imidazo[1,5-a]pyrrolo[2,3-e]pyrazin-1-yl)pyrrolidin-3-ol

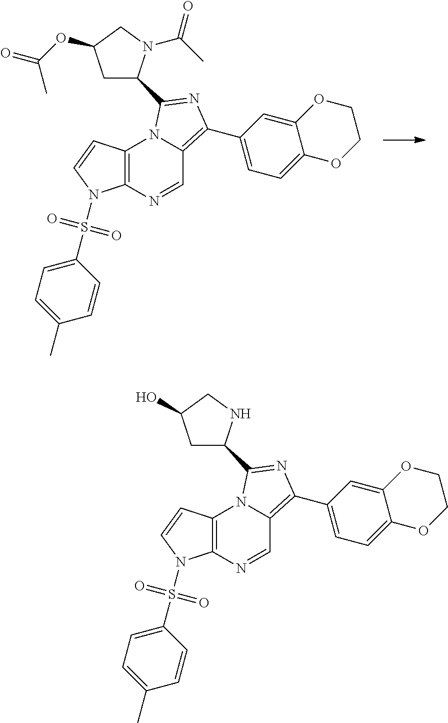 [SMILES: CC(=O)O[C@@H]1C[C@H](c2nc(-c3ccc4c(c3)OCCO4)c3cnc4c(ccn4S(=O)(=O)c4ccc(C)cc4)n23)N(C(C)=O)C1 ; Cc1ccc(S(=O)(=O)n2ccc3c2ncc2c(-c4ccc5c(c4)OCCO5)nc([C@H]4C[C@@H](O)CN4)n23)cc1]

A flask was charged with (3R,5R)-1-acetyl-5-(3-(chroman-6-yl)-6-tosyl-6H-imidazo[1,5-a]pyrrolo[2,3-e]pyrazin-1-yl)pyrrolidin-3-yl acetate (0.272 g, 0.443 mmol) and aqueous HCl (6 N, 2 mL, 12 mmol) in 1,4-dioxane (2 mL). The mixture was heated to about 75° C. for about 16 h. The mixture cooled to rt and saturated aqueous NaHCO₃ (10 mL) was added. The aqueous portion was extracted with a 10% solution of IPA in DCM (3×10 mL). The combined organic portions were dried over anhydrous MgSO4, filtered, and concentrated in vacuo to afford (3R,5R)-5-(3-(chroman-6-yl)-6-tosyl-6H-imidazo[1,5-a]pyrrolo[2,3-e]pyrazin-1-yl)pyrrolidin-3-ol (0.220 g, 78%), LC/MS (Table 3, Method a) Rₜ=1.93 min; MS m/z 530 (M+H)⁺.

### Step C: (2R,4R)-2-(3-(Chroman-6-yl)-6H-imidazo[1,5-a]pyrrolo[2,3-e]pyrazin-1-yl)-4-hydroxypyrrolidine-1-carboxamide

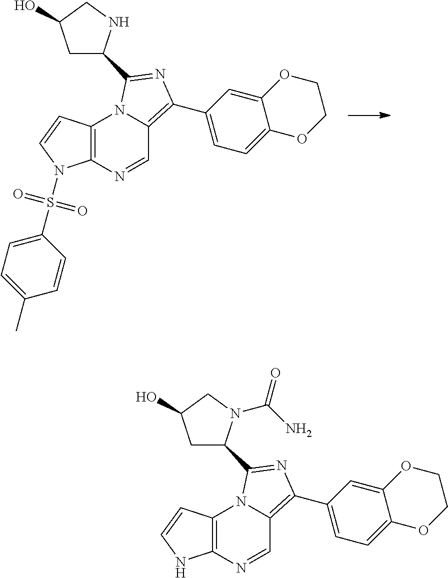 [SMILES: Cc1ccc(S(=O)(=O)n2ccc3c2ncc2c(-c4ccc5c(c4)OCCO5)nc([C@H]4C[C@@H](O)CN4)n23)cc1 ; NC(=O)N1C[C@H](O)C[C@@H]1c1nc(-c2ccc3c(c2)OCCO3)c2cnc3[nH]ccc3n12]

A flask was charged with (3R,5R)-5-(3-(chroman-6-yl)-6-tosyl-6H-imidazo[1,5-a]pyrrolo[2,3-e]pyrazin-1-yl)pyrrolidin-3-ol (0.22 g, 0.415 mmol), TEA (0.086 mL, 0.623 mmol), and isocyanatotrimethylsilane (0.223 mL, 1.662 mmol) in THF (1 mL). The mixture stirred at rt for about 3 h. The mixture was then partitioned between aqueous saturated NH₄Cl (10 mL) and DCM (10 mL). The aqueous portion was extracted with DCM (2×10 mL) and the combined organic portion was dried over MgSO₄, filtered, and concentrated in vacuo. The remaining residue was dissolved in a mixture of 1,4-dioxane (1 mL) and an aqueous solution of NaOH (1N, 2 mL, 2.0 mmol). The mixture was heated to about 75° C. for about 1 h. The mixture was cooled to rt and was partitioned between water (10 mL) and DCM (10 mL). The aqueous portion was extracted with a 10% solution of IPA in DCM (3×10 mL) dried over anhydrous MgSO₄, filtered, and concentrated in vacuo. The crude residue was purified by flash chromatography (silica gel; 0 to 10% MeOH in DCM) to give (2R,4R)-2-(3-(chroman-6-yl)-6H-imidazo[1,5-a]pyrrolo[2,3-e]pyrazin-1-yl)-4-hydroxypyrrolidine-1-carboxamide (0.059 g, 34%), LC/MS (Table 3, Method a) Rₜ=1.49 min; MS m/z 419 (M+H)⁺. Syk IC₅₀=B.

### Example #7

### 1-((2R,4R)-4-Hydroxy-2-(3-(4-(methoxymethyl)phenyl)-6H-imidazo[1,5-a]pyrrolo[2,3-e]pyrazin-1-yl)pyrrolidin-1-yl)ethanone

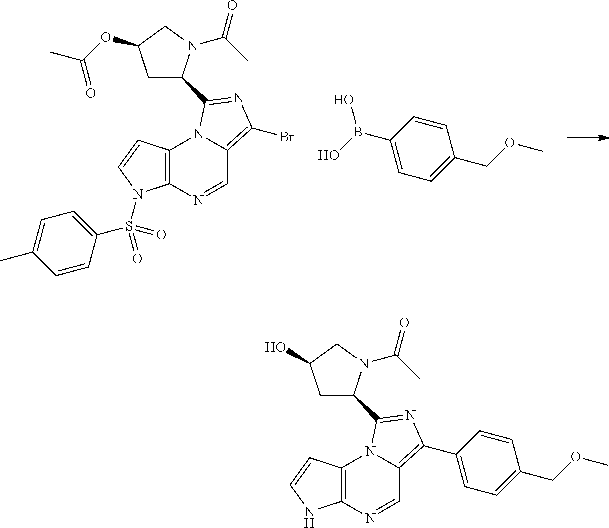 [SMILES: CC(=O)O[C@@H]1C[C@H](c2nc(Br)c3cnc4c(ccn4S(=O)(=O)c4ccc(C)cc4)n23)N(C(C)=O)C1 ; COCc1ccc(B(O)O)cc1 ; COCc1ccc(-c2nc([C@H]3C[C@@H](O)CN3C(C)=O)n3c2cnc2[nH]ccc23)cc1]

To a flask charged with (3R,5R)-1-acetyl-5-(3-bromo-6-tosyl-6H-imidazo[1,5-a]pyrrolo[2,3-e]pyrazin-1-yl)pyrrolidin-3-yl acetate (150 g, 268 mmol, Preparation #1), (4-(methoxymethyl)phenyl)boronic acid (53.3 g, 321 mmol), Cs₂CO₃ (131 g, 401 mmol), Pd(OAc)₂ (1.20 g, 5.35 mmol) and CgPPh (1.72 g, 5.89 mmol) was added toluene (300 mL) and water (30 mL). The mixture was heated to about 80° C. After about 4 h the mixture was cooled to rt and diluted with EtOAc (300 mL) and saturated aqueous NaHCO₃ (300 mL). The mixture was filtered and the organic layer was separated. The organic layer was mixed with saturated aqueous NaHCO₃ (300 mL), filtered, separated, washed with brine (300 mL) and concentrated in vacuo. The crude mixture was dissolved in EtOAc (900 mL) and mercaptopropyl functionalized silica gel (150 g, 180 mmol) was added. After stirring for about 15 h the mixture was filtered rinsing with EtOAc (3×500 mL). The filtrate was concentrated in vacuo. The residue was dissolved in a mixture of MeOH (300 mL). A solution of NaOH (64.2 g, 1606 mmol) and (R)-2-amino-3-mercaptopropanoic acid (64.9 g, 535 mmol) in water (600 mL) was added slowly. Following the addition the mixture was warmed to about 45° C. After about 15 h the mixture was partially concentrated in vacuo to remove most of the MeOH, diluted with water and the precipitate was collected by filtration, rinsed with water (5×500 mL). The wet cake was suspended in MeCN (1 L) and concentrated in vacuo 5 times. The solids were suspended in MeCN (˜1.5 L) with stirring. After about 10 h the solids were collected by filtration rinsing with MeCN and dried in vacuo. The solids were suspended in water (1 L) and MeOH (300 mL) and (R)-2-amino-3-mercaptopropanoic acid (64.9 g, 535 mmol) was added followed by a solution of aqueous NaOH (2.5 M, 800 mmol, 320 mL) was added slowly. After about 15 h the mixture was partially concentrated in vacuo removing ˜300 mL and filtered rinsing with water until a pH of 7 was obtained from the filtrate. The solids were dried in vacuo at about 50° C. to provide 1-((2R,4R)-4-hydroxy-2-(3-(4-(methoxymethyl)phenyl)-6H-imidazo[1,5-a]pyrrolo[2,3-e]pyrazin-1-yl)pyrrolidin-1-yl)ethanone (77 g, 71%): LC/MS (Table 3, Method a) Rₜ=1.63 min.; MS m/z: 406 (M+H)⁺. Syk IC₅₀=C.

### Example #8

### 1-((2R,4R)-2-(3-(4-((2H-1,2,3-Triazol-2-yl)methyl)phenyl)-6H-imidazo[1,5-a]pyrrolo[2,3-e]pyrazin-1-yl)-4-hydroxypyrrolidin-1-yl)ethanone

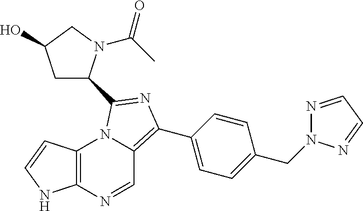 [SMILES: CC(=O)N1C[C@H](O)C[C@@H]1c1nc(-c2ccc(Cn3nccn3)cc2)c2cnc3[nH]ccc3n12]

### Step A: 2-(4-(4,4,5,5-Tetramethyl-1,3,2-dioxaborolan-2-yl)benzyl)-2H-1,2,3-triazole

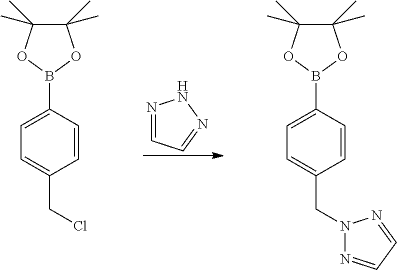

To a flask was added 2-(4-(chloromethyl)phenyl)-4,4,5,5-tetramethyl-1,3,2-dioxaborolane (0.310 g, 1.23 mmol, CombiBlocks), KI (0.031 g, 0.184 mmol) and K₂CO₃ (0.679 g, 4.91 mmol, JT Baker) in DMSO (5 mL) followed by 2H-1,2,3-triazole (0.170 g, 2.455 mmol, TCI). The mixture was heated at about 80° C. for about 1 h. The mixture was cooled to rt, filtered and concentrated in vacuo. The residue was purified by silica gel column eluting with 0 to 50% EtOAc in heptane, followed by a second column eluting with 0 to 50% EtOAc/heptane to provide 2-(4-(4,4,5,5-tetramethyl-1,3,2-dioxaborolan-2-yl)benzyl)-2H-1,2,3-triazole (0.055 g, 16%): LC/MS (Table 3, Method c) Rₜ=1.49 min; MS m/z: 286 (M+H)⁺.

### Step B: 1-((2R,4R)-2-(3-(4-((2H-1,2,3-Triazol-2-yl)methyl)phenyl)-6H-imidazo[1,5-a]pyrrolo[2,3-e]pyrazin-1-yl)-4-hydroxypyrrolidin-1-yl)ethanone

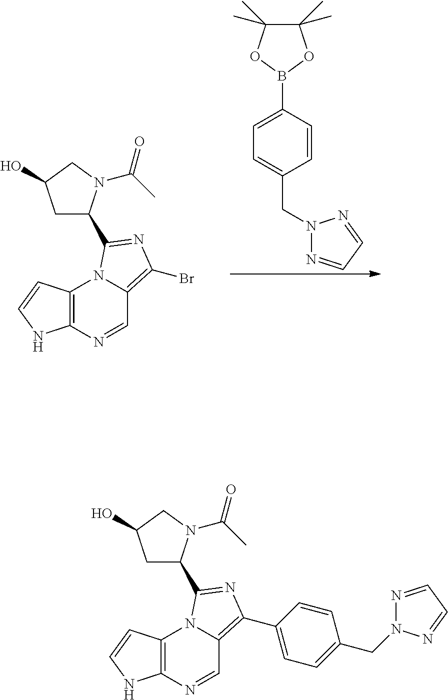 [SMILES: CC(=O)N1C[C@H](O)C[C@@H]1c1nc(Br)c2cnc3[nH]ccc3n12 ; CC1(C)OB(c2ccc(Cn3nccn3)cc2)OC1(C)C ; CC(=O)N1C[C@H](O)C[C@@H]1c1nc(-c2ccc(Cn3nccn3)cc2)c2cnc3[nH]ccc3n12]

To a flask was added 1-((2R,4R)-2-(3-bromo-6H-imidazo[1,5-a]pyrrolo[2,3-e]pyrazin-1-yl)-4-hydroxypyrrolidin-1-yl)ethanone (0.174 g, 0.478 mmol, Preparation #D.1), 2-(4-(4,4,5,5-tetramethyl-1,3,2-dioxaborolan-2-yl)benzyl)-2H-1,2,3-triazole (0.150 g, 0.526 mmol) and Cs₂CO₃ (0.390 g, 1.20 mmol) in water (1 mL) and 1,4-dioxane (4 mL) followed by PdCl₂(dppf) (0.035 g, 0.048 mmol). The mixture was heated to about 90° C. overnight. The mixture was diluted with EtOAc (20 mL) and washed with water (10 mL). The combined organic layers were dried over anhydrous MgSO₄, filtered and concentrated. The crude material was purified by silica gel column eluting with 0 to 10% MeOH in DCM to give 1-((2R,4R)-2-(3-(4-((2H-1,2,3-triazol-2-yl)methyl)phenyl)-6H-imidazo[1,5-a]pyrrolo[2,3-e]pyrazin-1-yl)-4-hydroxypyrrolidin-1-yl)ethanone (0.040 g, 19%): LC/MS (Table 3, Method a) Rₜ=1.31 min; MS m/z: 443(M+H)⁺. Syk IC₅₀=C.

### Example #9

### 1-((2R,4R)-4-Hydroxy-2-(3-(4-(2,2,2-trifluoroethoxy)phenyl)-6H-imidazo[1,5-a]pyrrolo[2,3-e]pyrazin-1-yl)pyrrolidin-1-yl)ethanone

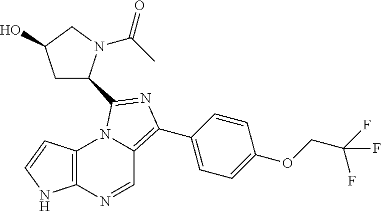 [SMILES: CC(=O)N1C[C@H](O)C[C@@H]1c1nc(-c2ccc(OCC(F)(F)F)cc2)c2cnc3[nH]ccc3n12]

To a 5 mL microwave reaction vial was added (3R,5R)-1-acetyl-5-(3-bromo-6-tosyl-6H-imidazo[1,5-a]pyrrolo[2,3-e]pyrazin-1-yl)pyrrolidin-3-yl acetate (0.1 g, 0.178 mmol, Preparation #1), 4-(2,2,2-trifluoroethoxy)phenylboronic acid (0.086 g, 0.393 mmol, Combi-Blocks), SiliaCat® diphenylphosphinopropane-Pd (0.074 g, 0.018 mmol) in 1,4-dioxane (1.034 ml) and EtOH (1.034 ml). A solution of Cs₂CO₃ (0.174 g, 0.535 mmol) in water (0.310 ml) was added, the mixture was heated in a microwave at about 150° C. (250 psi maximum pressure, 10 min ramp, 300 max watts) for about 30 min. The mixture was cooled to rt, filtered through Celite® and dried over anhydrous MgSO₄. The crude material was purified via silica gel chromatography (0 to 10% MeOH in DCM) to afford 1-((2R,4R)-4-hydroxy-2-(3-(4-(2,2,2-trifluoroethoxy)phenyl)-6H-imidazo[1,5-a]pyrrolo[2,3-e]pyrazin-1-yl)pyrrolidin-1-yl)ethanone (0.067 g, 81%): LC/MS (Table 3, Method a) Rₜ=1.80 min; MS m/z: 460 (M+H)⁺. Syk IC₅₀=C.

### Example #10

### 1-((2R,4R)-4-Hydroxy-2-(3-(4-(isopropylamino)phenyl)-6H-imidazo[1,5-a]pyrrolo[2,3-e]pyrazin-1-yl)pyrrolidin-1-yl)ethanone

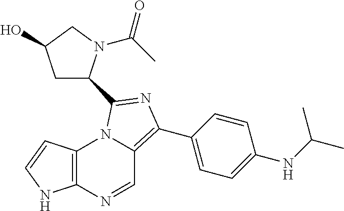 [SMILES: CC(=O)N1C[C@H](O)C[C@@H]1c1nc(-c2ccc(NC(C)C)cc2)c2cnc3[nH]ccc3n12]

### Step A: (3R,5R)-1-Acetyl-5-(3-(4-(isopropylamino)phenyl)-6-tosyl-6H-imidazo[1,5-a]pyrrolo[2,3-e]pyrazin-1-yl)pyrrolidin-3-yl acetate

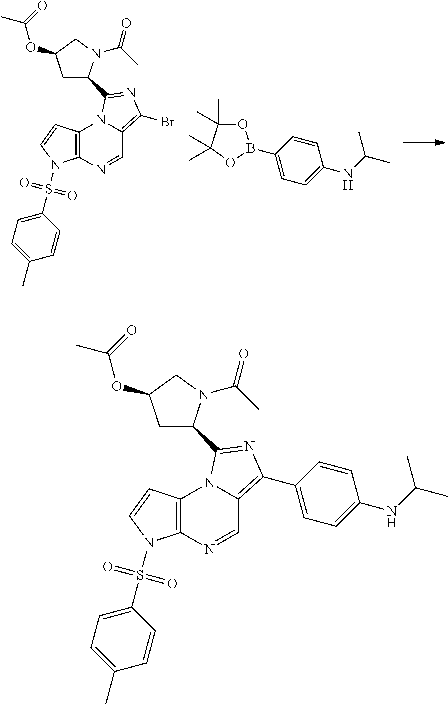

(3R,5R)-1-Acetyl-5-(3-bromo-6-tosyl-6H-imidazo[1,5-a]pyrrolo[2,3-e]pyrazin-1-yl)pyrrolidin-3-yl acetate (0.15 g, 0.268 mmol, Preparation #1), N-isopropyl-4-(4,4,5,5-tetramethyl-1,3,2-dioxaborolan-2-yl)aniline (0.140 g, 0.535 mmol, Combi-Blocks), and TEA (0.112 ml, 0.803 mmol) in DME (2.86 mL) and water (0.714 mL) were added to a flask. The mixture was sparged with nitrogen for about 10 min and then PdCl₂(dppf) (0.022 g, 0.027 mmol) was added. The mixture was evacuated and purged with nitrogen three times and then heated at about 80° C. for about 16 h. The mixture was cooled to rt and then filtered through Celite®, dried over anhydrous MgSO₄, filtered and concentrated in vacuo. The crude material was purified via silica gel chromatography (0 to 10% MeOH in DCM) to afford (3R,5R)-1-acetyl-5-(3-(4-(isopropylamino)phenyl)-6-tosyl-6H-imidazo[1,5-a]pyrrolo[2,3-e]pyrazin-1-yl)pyrrolidin-3-yl acetate (0.165 g, 100%): LC/MS (Table 3, Method a) Rₜ=2.38 min; MS m/z: 615 (M+H)⁺.

### Step B: 1-((2R,4R)-4-Hydroxy-2-(3-(4-(isopropylamino)phenyl)-6H-imidazo[1,5-a]pyrrolo[2,3-e]pyrazin-1-yl)pyrrolidin-1-yl)ethanone

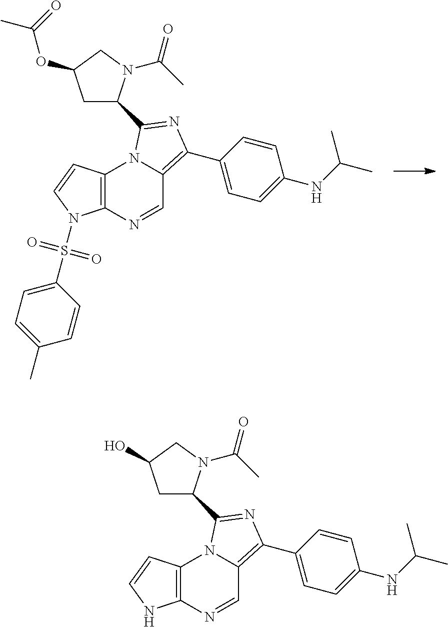 [SMILES: CC(=O)O[C@@H]1C[C@H](c2nc(-c3ccc(NC(C)C)cc3)c3cnc4c(ccn4S(=O)(=O)c4ccc(C)cc4)n23)N(C(C)=O)C1 ; CC(=O)N1C[C@H](O)C[C@@H]1c1nc(-c2ccc(NC(C)C)cc2)c2cnc3[nH]ccc3n12]

To a solution of (3R,5R)-1-acetyl-5-(3-(4-(isopropylamino)phenyl)-6-tosyl-6H-imidazo[1,5-a]pyrrolo[2,3-e]pyrazin-1-yl)pyrrolidin-3-yl acetate (0.165 g, 0.268 mmol) in THF (2.0 mL) was added Cs₂CO₃ (0.262 g, 0.804 mmol). The mixture was heated in a microwave at about 150° C. (250 psi maximum pressure, 10 min ramp, 300 max watts) for about 30 min. The mixture was cooled to rt and filtered through Celite®, dried over anhydrous MgSO₄, filtered and concentrated in vacuo. The crude material was purified via silica gel chromatography (0 to 10% MeOH in DCM) to afford 1-((2R,4R)-4-hydroxy-2-(3-(4-(isopropylamino)phenyl)-6H-imidazo[1,5-a]pyrrolo[2,3-e]pyrazin-1-yl)pyrrolidin-1-yl)ethanone (0.030 g, 25%): LC/MS (Table 3, Method a) Rₜ=1.66 min; MS m/z: 419 (M+H)⁺. Syk IC₅₀=B.

### Example #11

### 1-((2R,4R)-4-Hydroxy-2-(3-(4-(isopropylamino)phenyl)-8-methyl-6H-imidazo[1,5-a]pyrrolo[2,3-e]pyrazin-1-yl)pyrrolidin-1-yl)ethanone

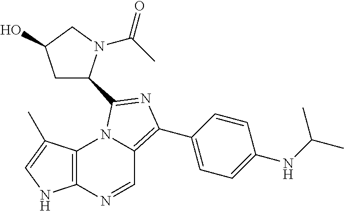 [SMILES: CC(=O)N1C[C@H](O)C[C@@H]1c1nc(-c2ccc(NC(C)C)cc2)c2cnc3[nH]cc(C)c3n12]

### Step A: Benzyl (4-(1-((2R,4R)-1-acetyl-4-hydroxypyrrolidin-2-yl)-8-methyl-6H-imidazo[1,5-a]pyrrolo[2,3-e]pyrazin-3-yl)phenyl)(isopropyl)carbamate

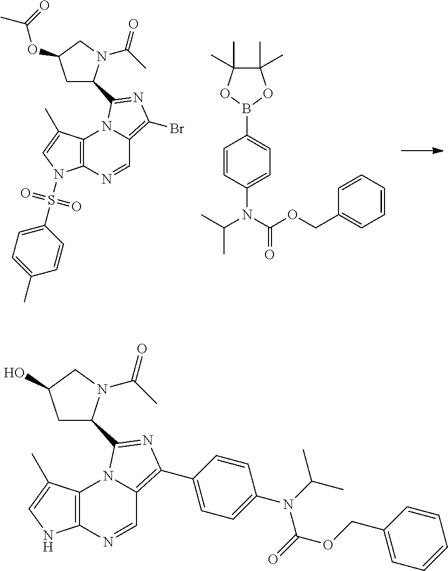 [SMILES: CC(=O)O[C@@H]1C[C@H](c2nc(Br)c3cnc4c(c(C)cn4S(=O)(=O)c4ccc(C)cc4)n23)N(C(C)=O)C1 ; CC(C)N(C(=O)OCc1ccccc1)c1ccc(B2OC(C)(C)C(C)(C)O2)cc1 ; CC(=O)N1C[C@H](O)C[C@@H]1c1nc(-c2ccc(N(C(=O)OCc3ccccc3)C(C)C)cc2)c2cnc3[nH]cc(C)c3n12]

To a 5 mL reaction vial was added (3R,5R)-1-acetyl-5-(3-bromo-8-methyl-6-tosyl-6H-imidazo[1,5-a]pyrrolo[2,3-e]pyrazin-1-yl)pyrrolidin-3-yl acetate (0.25 g, 0.435 mmol, Preparation #2), benzyl isopropyl(4-(4,4,5,5-tetramethyl-1,3,2-dioxaborolan-2-yl)phenyl)carbamate (0.430 g, 1.09 mmol, Combi-Blocks), and SiliaCat® diphenylphosphinopropane-Pd (0.181 g, 0.044 mmol) (Silicycle) in 1,4-dioxane (2.52 ml) and EtOH (2.52 ml). A solution of Cs₂CO₃ (0.425 g, 1.31 mmol) in water (0.757 ml) was added. The mixture was heated in a microwave at about 150° C. (250 psi maximum pressure, 10 min ramp, 300 max watts) for about 30 min. The mixture was cooled to rt and then was purified via silica gel chromatography (0 to 10% MeOH in DCM) to afford to afford benzyl 4-(1-((2R,4R)-1-acetyl-4-hydroxypyrrolidin-2-yl)-8-methyl-6H-imidazo[1,5-a]pyrrolo[2,3-e]pyrazin-3-yl)phenyl(isopropyl)carbamate (0.17 g, 69%). LC/MS (Table 3, Method a) Rₜ=2.12 min; MS m/z: 567 (M+H)⁺.

### Step B: 1-((2R,4R)-4-Hydroxy-2-(3-(4-(isopropylamino)phenyl)-8-methyl-6H-imidazo[1,5-a]pyrrolo[2,3-e]pyrazin-1-yl)pyrrolidin-1-yl)ethanone

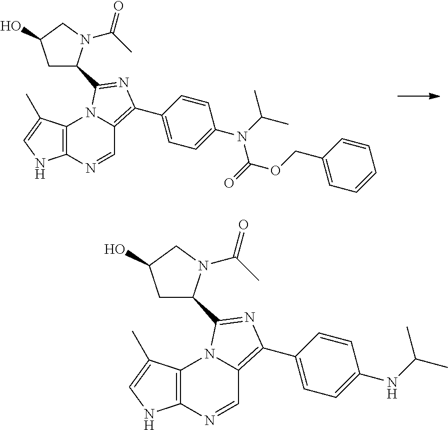 [SMILES: CC(=O)N1C[C@H](O)C[C@@H]1c1nc(-c2ccc(N(C(=O)OCc3ccccc3)C(C)C)cc2)c2cnc3[nH]cc(C)c3n12 ; CC(=O)N1C[C@H](O)C[C@@H]1c1nc(-c2ccc(NC(C)C)cc2)c2cnc3[nH]cc(C)c3n12]

To a flask was added benzyl 4-(1-((2R,4R)-1-acetyl-4-hydroxypyrrolidin-2-yl)-8-methyl-6H-imidazo[1,5-a]pyrrolo[2,3-e]pyrazin-3-yl)phenyl(isopropyl)carbamate (0.17 g, 0.300 mmol), MeCN (3.00 mL) and TMSI (0.408 mL, 3.00 mmol). The mixture was stirred at rt for about 18 hr. The mixture was diluted with DCM (50 mL), washed by 10% sodium hydrosulfite (30 mL) and saturated aqueous NaHCO₃ (30 mL), brine (20 mL), dried over Na₂SO₄, filtered and concentrated in vacuo. The residue was purified via silica gel chromatography (0 to 10% MeOH in DCM) to afford 1-((2R,4R)-4-hydroxy-2-(3-(4-(isopropylamino)phenyl)-8-methyl-6H-imidazo[1,5-a]pyrrolo[2,3-e]pyrazin-1-yl)pyrrolidin-1-yl)ethanone (0.1 g, 75%). LC/MS (Table 3, Method a) Rₜ=1.78 min; MS m/z: 433 (M+H)⁺. Syk IC₅₀=C.

Compounds of the invention are selective for Syk.

What is claimed:

1. A compound of Formula (I) 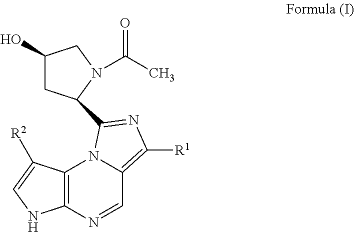
or a pro-drug, metabolite, isomer, stereoisomer or pharmaceutically acceptable salt thereof wherein
R¹ is phenyl, 2,3-dihydorbenzo[b][1,4]dioxine, thiazolyl, thienyl, or 1,2,3,4-tetrahydroquinolinyl, wherein
the phenyl is substituted by one or more substituents independently selected from —C(CH₃)₂OH, —OCH₃, —C(CH₃)₂CN, —OCH₂CH₂OCH₃, —OCH₂CH₂-morpholinyl, —CH₃, morpholinyl, F, —OCH(CH₃)₂, —OCF₃, —OCH₂CF₃, 1-hydroxycyclobuty, —OCH₂-cyclopropyl, tetrahydropyranyl, —CH₂CN, —CHF₂, CF₃, piperidinyl, —N(H)CH(CH₃)₂, 2-methylmorpholinyl, 4,4-difluoropiperidinyl, 4,4-difluoroazetidinyl, —C(O)-4,4-difluropiperidinyl, —N(H)CH₃, —N(H)CH₂CH₃, —C(H)(CH₃)OCH₃, —OCH₂CH₂-triazolyl, —OCH₂-3-methyloxetanyl, —CH₂— triazolyl or —OCH(CH₃)-cyclopropyl; or
the thienyl is substituted by —OCH₃ or —CH₂OH; or
the thiazolyl is substituted by tert-butyl; and
R² is H, Cl or CH₃.

2. The compound of claim 1, wherein the compound is:
1-((2R,4R)-4-hydroxy-2-(3-(4-(2-hydroxypropan-2-yl)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-methoxyphenyl)-6H-imidazo[1,5-a]pyrrolo[2,3-e]pyrazin-1-yl)pyrrolidin-1-yl)ethanone;
1-((2R,4R)-4-hydroxy-2-(3-(4-methoxy-3-methylphenyl)-6H-imidazo[1,5-a]pyrrolo[2,3-e]pyrazin-1-yl)pyrrolidin-1-yl)ethanone;
2-(4-(1-((2R,4R)-1-acetyl-4-hydroxypyrrolidin-2-yl)-6H-imidazo[1,5-a]pyrrolo[2,3-e]pyrazin-3-yl)phenyl)-2-methylpropanenitrile;
1-((2R,4R)-2-(3-(3,4-dimethoxyphenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(2-methoxyethoxy)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-(2-morpholinoethoxy)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-morpholinophenyl)-6H-imidazo[1,5-a]pyrrolo[2,3-e]pyrazin-1-yl)pyrrolidin-1-yl)ethanone;
1-((2R,4R)-2-(3-(3-fluoro-4-(2-hydroxypropan-2-yl)phenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-isopropoxyphenyl)-6H-imidazo[1,5-a]pyrrolo[2,3-e]pyrazin-1-yl)pyrrolidin-1-yl)ethanone;
1-((2R,4R)-2-(3-(benzo[d][1,3]dioxol-5-yl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(trifluoromethoxy)phenyl)-6H-imidazo[1,5-a]pyrrolo[2,3-e]pyrazin-1-yl)pyrrolidin-1-yl)ethanone;
1-((2R,4R)-2-(3-(3-fluoro-4-methoxyphenyl)-6H-imidazo[1,5-a]pyrrolo[2,3-e]pyrazin-1-yl-4-hydroxypyrrolidin-1-yl)ethanone;
1-((2R,4R)-4-hydroxy-2-(3-(4-(2,2,2-trifluoroethoxy)phenyl)-6H-imidazo[1,5-a]pyrrolo[2,3-e]pyrazin-1-yl)pyrrolidin-1-yl)ethanone;
1-((2R,4R)-4-hydroxy-2-(3-(5-methoxythiophen-2-yl)-6H-imidazo[1,5-a]pyrrolo[2,3-e]pyrazin-1-yl)pyrrolidin-1-yl)ethanone;
1-((2R,4R)-4-hydroxy-2-(3-(4-(1-hydroxycyclobutyl)phenyl)-6H-imidazo[1,5-a]pyrrolo[2,3-e]pyrazin-1-yl)pyrrolidin-1-yl)ethanone;
1-((2R,4R)-2-(3-(4-(cyclopropylmethoxy)phenyl)-6H-imidazo[1,5-a]pyrrolo[2,3-e]pyrazin-1-yl)-4-hydroxypyrrolidin-1-yl)ethanone;
1-((2R,4R)-2-(3-(4-ethoxy-3-methylphenyl)-6H-imidazo[1,5-a]pyrrolo[2,3-e]pyrazin-1-yl)-4-hydroxypyrrolidin-1-yl)ethanone;
1-((2R,4R)-2-(3-(4-ethoxyphenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(methoxymethyl)phenyl)-6H-imidazo[1,5-a]pyrrolo[2,3-e]pyrazin-1-yl)pyrrolidin-1-yl)ethanone;
1-((2R,4R)-2-(3-(2-(tert-Butyl)thiazol-5-yl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(tetrahydro-2H-pyran-4-yl)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-((3-methyloxetan-3-yl)methoxy)phenyl)-6H-imidazo[1,5-a]pyrrolo[2,3-e]pyrazin-1-yl)pyrrolidin-1-yl)ethanone;
1-((2R,4R)-2-(3-(3,4-dihydro-2H-benzo[b][1,4]oxazin-6-yl)-6H-imidazo[1,5-a]pyrrolo[2,3-e]pyrazin-1-yl)-4-hydroxypyrrolidin-1-yl)ethanone;
1-((2R,4R)-2-(3-(2,3-dihydrobenzo[b][1,4]dioxin-6-yl)-6H-imidazo[1,5-a]pyrrolo[2,3-e]pyrazin-1-yl)-4-hydroxypyrrolidin-1-yl)ethanone;
1-((2S,4S)-4-hydroxy-2-(3-(thiophen-3-yl)-6H-imidazo[1,5-a]pyrrolo[2,3-e]pyrazin-1-yl)pyrrolidin-1-yl)ethanone;
2-(4-(1-((2S,4S)-1-acetyl-4-hydroxypyrrolidin-2-yl)-6H-imidazo[1,5-a]pyrrolo[2,3-e]pyrazin-3-yl)phenyl)acetonitrile;
1-((2R,4R)-4-hydroxy-2-(3-(1,2,3,4-tetrahydroquinolin-7-yl)-6H-imidazo[1,5-a]pyrrolo[2,3-e]pyrazin-1-yl)pyrrolidin-1-yl)ethanone;
1-((2R,4R)-2-(3-(chroman-6-yl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(piperidin-1-yl)phenyl)-6H-imidazo[1,5-a]pyrrolo[2,3-e]pyrazin-1-yl)pyrrolidin-1-yl)ethanone;
1-((2R,4R)-2-(3-(4-(difluoromethyl)phenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(trifluoromethyl)phenyl)-6H-imidazo[1,5-a]pyrrolo[2,3-e]pyrazin-1-yl)pyrrolidin-1-yl)ethanone;
1-((2R,4R)-2-(8-chloro-3-(4-morpholinophenyl)-6H-imidazo[1,5-a]pyrrolo[2,3-e]pyrazin-1-yl)-4-hydroxypyrrolidin-1-yl)ethanone;
1-((2R,4R)-2-(8-chloro-3-(4-ethoxyphenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(isopropylamino)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-(2-methylmorpholino)phenyl)-6H-imidazo[1,5-a]pyrrolo[2,3-e]pyrazin-1-yl)pyrrolidin-1-yl)ethanone;
1-((2R,4R)-2-(3-(4-(4,4-difluoropiperidin-1-yl)phenyl)-6H-imidazo[1,5-a]pyrrolo[2,3-e]pyrazin-1-yl)-4-hydroxypyrrolidin-1-yl)ethanone;
1-((2R,4R)-2-(3-(4-(3,3-difluoroazetidin-1-yl)phenyl)-6H-imidazo[1,5-a]pyrrolo[2,3-e]pyrazin-1-yl)-4-hydroxypyrrolidin-1-yl)ethanone;
(2R,4R)-2-(3-(2,3-Dihydrobenzo[b][1,4]dioxin-6-yl)-6H-imidazo[1,5-a]pyrrolo[2,3-e]pyrazin-1-yl)-4-hydroxypyrrolidine-1-carboxamide;
2-fluoro-1-((2R,4R)-4-hydroxy-2-(3-(4-(2-methoxyethoxy)phenyl)-6H-imidazo[1,5-a]pyrrolo[2,3-e]pyrazin-1-yl)pyrrolidin-1-yl)ethanone;
1-((2R,4R)-2-(3-(4-(4,4-difluoropiperidine-1-carbonyl)phenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-(isopropylamino)phenyl)-8-methyl-6H-imidazo[1,5-a]pyrrolo[2,3-e]pyrazin-1-yl)pyrrolidin-1-yl)ethanone;
1-((2R,4R)-4-hydroxy-2-(3-(4-(methylamino)phenyl)-6H-imidazo[1,5-a]pyrrolo[2,3-e]pyrazin-1-yl)pyrrolidin-1-yl)ethanone;
1-((2R,4R)-2-(3-(4-(ethylamino)phenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-((R)-1-methoxyethyl)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-((S)-1-methoxyethyl)phenyl)-6H-imidazo[1,5-a]pyrrolo[2,3-e]pyrazin-1-yl)pyrrolidin-1-yl)ethanone;
1-((2R,4R)-4-hydroxy-2-(3-(5-(hydroxymethyl)thiophen-3-yl)-6H-imidazo[1,5-a]pyrrolo[2,3-e]pyrazin-1-yl)pyrrolidin-1-yl)ethanone;
1-((2R,4R)-2-(3-(4-(2-(2H-1,2,3-triazol-2-yl)ethoxy)phenyl)-6H-imidazo[1,5-a]pyrrolo[2,3-e]pyrazin-1-yl)-4-hydroxypyrrolidin-1-yl)ethanone;
1-((2R,4R)-4-hydroxy-2-(3-(4-((S)-2-methylmorpholino)phenyl)-6H-imidazo[1,5-a]pyrrolo[2,3-e]pyrazin-1-yl)pyrrolidin-1-yl)ethanone;
1-((2R,4R)-4-hydroxy-2-(3-(4-((R)-2-methylmorpholino)phenyl)-6H-imidazo[1,5-a]pyrrolo[2,3-e]pyrazin-1-yl)pyrrolidin-1-yl)ethanone;
1-((2R,4R)-2-(3-(4-((2H-1,2,3-triazol-2-yl)methyl)phenyl)-6H-imidazo[1,5-a]pyrrolo[2,3-e]pyrazin-1-yl)-4-hydroxypyrrolidin-1-yl)ethanone; or,
1-((2R,4R)-2-(3-(4-((R)-1-cyclopropylethoxy)phenyl)-6H-imidazo[1,5-a]pyrrolo[2,3-e]pyrazin-1-yl)-4-hydroxypyrrolidin-1-yl)ethanone.

3. A method of treating a disease comprising administering a therapeutically effective amount of a compound of Formula (I) to a patient in need thereof.

4. The method according to claim 3 wherein the disease is rheumatoid arthritis, juvenile rheumatoid arthritis, osteoarthritis, Crohn's disease, inflammatory bowel disease, ankylosing spondylitis, interstitial cystitis, asthma, systemic lupus erythematosus or multiple sclerosis.

5. A kit comprising a packaged product comprising components with which to administer a compound of Formula (I) of the invention for treatment of an autoimmune disorder.

6. The kit according to claim 5 wherein the packaged product comprises a compound of Formula (I) and instructions for use.

7. A pharmaceutical composition comprising a compound according to claim 1 and one or more pharmaceutically acceptable excipients.
